(19) United States
(12) Patent Application Publication
(10) Pub. No.: US 2022/0288214 A1
(43) Pub. Date: Sep. 15, 2022

(54) CONJUGATES AND METHODS FOR TREATING ACROMEGALY

(71) Applicant: GENEVANT SCIENCES GMBH, Basel (CH)

(72) Inventors: Drew KONDRATOWICZ, Vancouver (CA); James HEYES, Vancouver (CA); Richard J. HOLLAND, Vancouver (CA); Christine ESAU, Vancouver (CA); Kevin MCCLINTOCK, Vancouver (CA); Christopher Justin PASETKA, Langley (CA); Mark WOOD, Moody (CA); Alan D. MARTIN, Vancouver (CA); Margrit SCHWARZ, Stow, MA (US); David CROWE, Vancouver (CA); Steven TYLER, Vancouver (CA)

(73) Assignee: GENEVANT SCIENCES GMBH, Basel (CH)

(21) Appl. No.: 17/770,504

(22) PCT Filed: Oct. 23, 2020
(86) PCT No.: PCT/US2020/057185

§ 371 Date: Apr. 20, 2022

Related U.S. Application Data
(60) Provisional application No. 62/925,659, filed on Oct. 24, 2019.

(51) Int. Cl. A61K 47/54; A61P 5/08; C12N 15/113

(52) U.S. Cl. CPC A61K 47/549; A61P 5/08; C12N 15/1136; C12N 2310/14; C12N 2310/351; C12N 2320/30

(57) ABSTRACT

Provided herein are certain nucleic acids (e.g., double stranded siRNA molecules), as well as conjugates that comprise a targeting moiety, a double stranded siRNA, and optional linking groups. Certain embodiments also provide synthetic methods useful for preparing the conjugates. He conjugates are useful to treat certain diseases, such as acromegaly.

### CROSS-REFERENCE TO RELATED APPLICATION(S)

This patent application claims the benefit of priority of U.S. application Ser. No. 62/925,659, filed Oct. 24, 2019, which application is herein incorporated by reference.

### BACKGROUND

Acromegaly is a condition caused by the hypersecretion of growth hormone (GH), which results in abnormal skeletal, tissue, and organ growth. Untreated acromegaly leads to reduced life expectancy, with the vast majority of the 36-60 million cases dying from cardiovascular disease. There are several therapeutic options available for acromegaly, ranging from pharmacological intervention to the surgical removal of the pituitary tumor that triggers the disease. However, response rates vary and usually require multiple therapeutics and negative side-effects. Accordingly, new therapeutic treatment options are needed.

### BRIEF SUMMARY

Nucleic acid (e.g., siRNA) therapy is one approach for the treatment of GH hypersecretion via the reduction of growth hormone receptor (GHR) in the liver, thus preventing the down-stream signaling cascade that leads to the disease. Described herein is the hepatocyte-specific delivery of siRNA targeting the GHR transcript, which is a useful treatment option. This reduction in the transcript and protein will prevent growth hormone-derived signaling, and therefore reduce insulin-like growth factor-1 (IGF-1), which is the main etiological agent of the disease. This solution confers an advantage compared to other treatments options due to the ease of administration, which includes the duration of effect, and the expected safety profile.

In certain embodiments, provided herein are nucleic acid molecules (e.g., therapeutic double stranded siRNA molecules), as well as conjugates, compositions and methods that can be used to deliver such nucleic acids.

Accordingly, one aspect provides a double stranded siRNA molecule selected from the group consisting of siRNA 1-siRNA 28.

Another aspect provides a compound of formula I

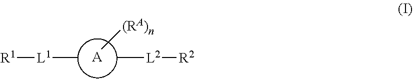

wherein:

R¹ a is targeting ligand;

L¹ is absent or a linking group;

L² is absent or a linking group;

R² is a double stranded siRNA molecule selected from the double stranded siRNA of Table 1 and Table 2;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

Another aspect provides GalNAc conjugates that comprise one of the siRNAs described herein, which conjugates are not limited to conjugates that comprise the ligand-linkers disclosed herein. For example, an aspect provides a GalNAc conjugate of Formula X:

A-B-C (X)

wherein A is a targeting ligand;
B is an optional linker; and
C is an siRNA molecule described herein.

Additional conjugates useful with the siRNA molecules described herein are described in WO 2017/177326 (PCT/CA2017/050447) and in WO 2018/191278 (PCT/US2018/026918), the disclosures of which are each incorporated by reference.

The therapeutic double stranded siRNA described herein, as well as, compounds and compositions comprising such siRNA, may be used to treat Hepatitis B virus and Hepatitis B virus/Hepatitis D virus.

Provided herein are also synthetic intermediates and methods disclosed herein that are useful to prepare compounds of formula I.

Other objects, features, and advantages will be apparent to one of skill in the art from the following detailed description and figures.

### BRIEF DESCRIPTION OF THE FIGURES

FIG. 1 depicts the dose-response of 24 GalNAc-conjugated Human GHR targeting candidates in PHHs. Increasing concentrations of each candidate were incubated with primary human hepatocytes for 48 hours, with delivery being GalNAc-dependent. GHR mRNA was assayed by qPCR.

FIG. 2 depicts liver injury markers after a single-dose of GHR-targeting candidates. Male rats received a single sub-cutaneous injection of the indicated candidate at 20 or 60 mg/kg. Serum markers of liver injury were analyzed 14-days post-dose. Saline is presented on the far left of each graph. A conjugate of siRNA 25 is presented as the left data set for each dose. A conjugate of siRNA 27 is presented as the right data set for each dose.

FIG. 3 depicts GHR mRNA reduction in NHPs after a single administration of candidates. Cynomolgus macaques were administered the indicated dosage of each clinical candidate subcutaneously. 14-days post-dose, liver biopsies were taken and GHR mRNA levels were assayed by qPCR. Saline is presented on the far left of the graph. A conjugate of siRNA 25 is presented as the left data set. A conjugate of siRNA 27 is presented as the right data set.

FIG. 4 depicts comparative data between a conjugate of siRNA 25 as described in the current application (lower trace) with a GalNAc-ASO (a triantennary N-acetyl galactosamine-antisense oligonucleotide conjugate) from Ionis Pharmaceuticals, Inc. (upper trace). As depicted in the figure, the conjugate of siRNA 25 displayed improved properties.

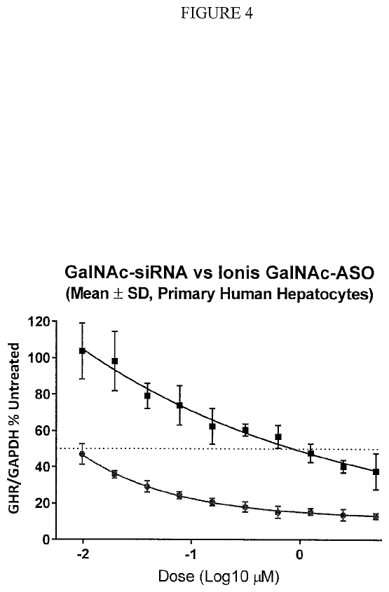

FIG. 5 depicts comparative data between a conjugate of siRNA 27 as described in the current application (lower trace with squares; PHH: lower trace with circles; PMH) with a GalNAc-ASO (a triantennary N-acetyl galactosamine-antisense oligonucleotide conjugate) from Ionis Pharmaceuticals, Inc. (upper trace with squares; PHH: upper trace with circles; PMH). As depicted in the figure, the conjugate of siRNA 27 displayed improved properties.

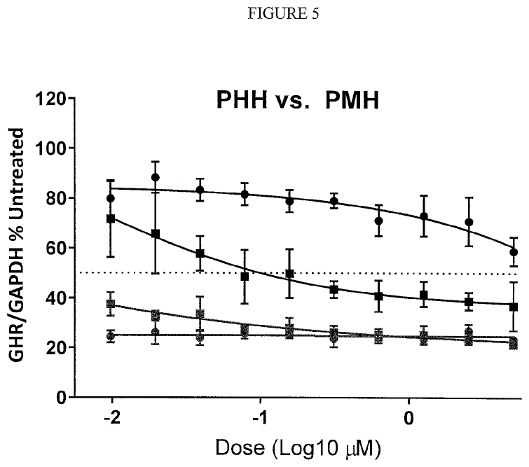

FIG. 6 depicts comparative data between conjugates of siRNA 25 (lower trace) and siRNA 27 (middle trace) as described in the current application with a GalNAc-ASO (a triantennary N-acetyl galactosamine-antisense oligonucleotide conjugate) from Ionis Pharmaceuticals, Inc. (upper trace). As depicted in the figure, the conjugates of siRNAs 25 and 27 displayed improved properties.

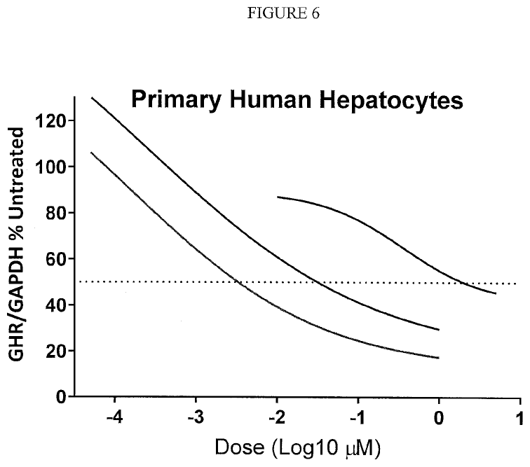

In the application, including Figures, Examples and Schemes, it is to be understood that an oligonucleotide can be a double stranded siRNA molecule as described in Table 1 or Table 2.

### DETAILED DESCRIPTION

Accordingly, provided herein is a compound of formula (I):

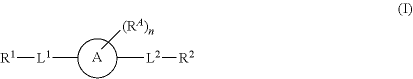

wherein:

R¹ a is targeting ligand;

L¹ is absent or a linking group;

L² is absent or a linking group;

R² is a siRNA molecule selected from any one of siRNA 1-siRNA 28;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen or a protecting group; and

n is 0, 1, 2, 3, 4, 5, 6, 7, 8, 9, or 10;

or a salt thereof.

In certain embodiments, R¹ is —C(H)₍₃₋ₚ₎(L³-saccharide)ₚ;

wherein each L³ is independently a linking group;

p is 1, 2, or 3; and

saccharide is a monosaccharide or disaccharide

or a salt thereof.

In certain embodiments, the saccharide is:

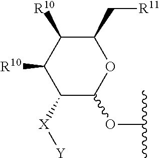

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;

or a salt thereof.

In certain embodiments, the saccharide is selected from the group consisting of:

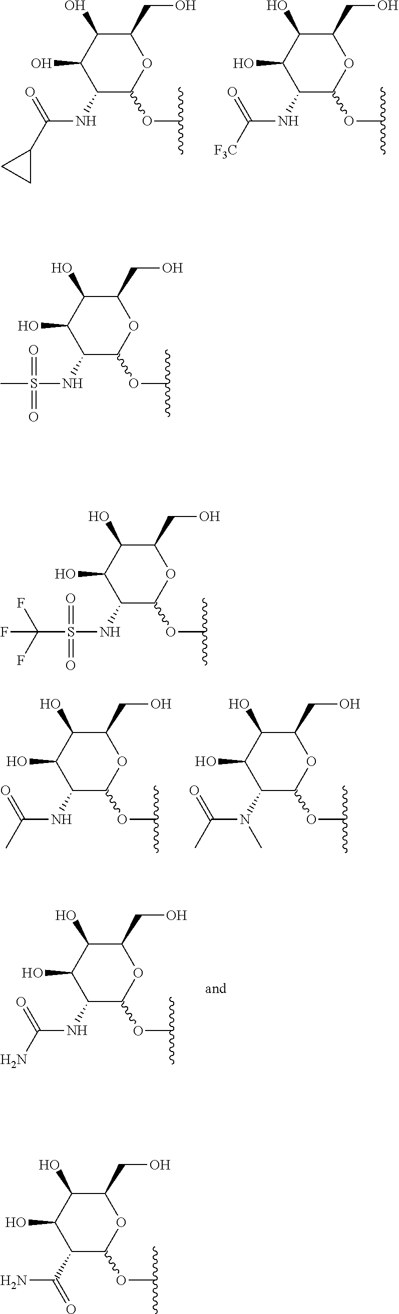

or a salt thereof.

In certain embodiments, the saccharide is:

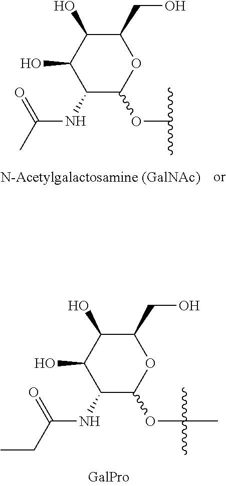 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

or a salt thereof.

In certain embodiments, the compound of formula I is selected from the group consisting of:

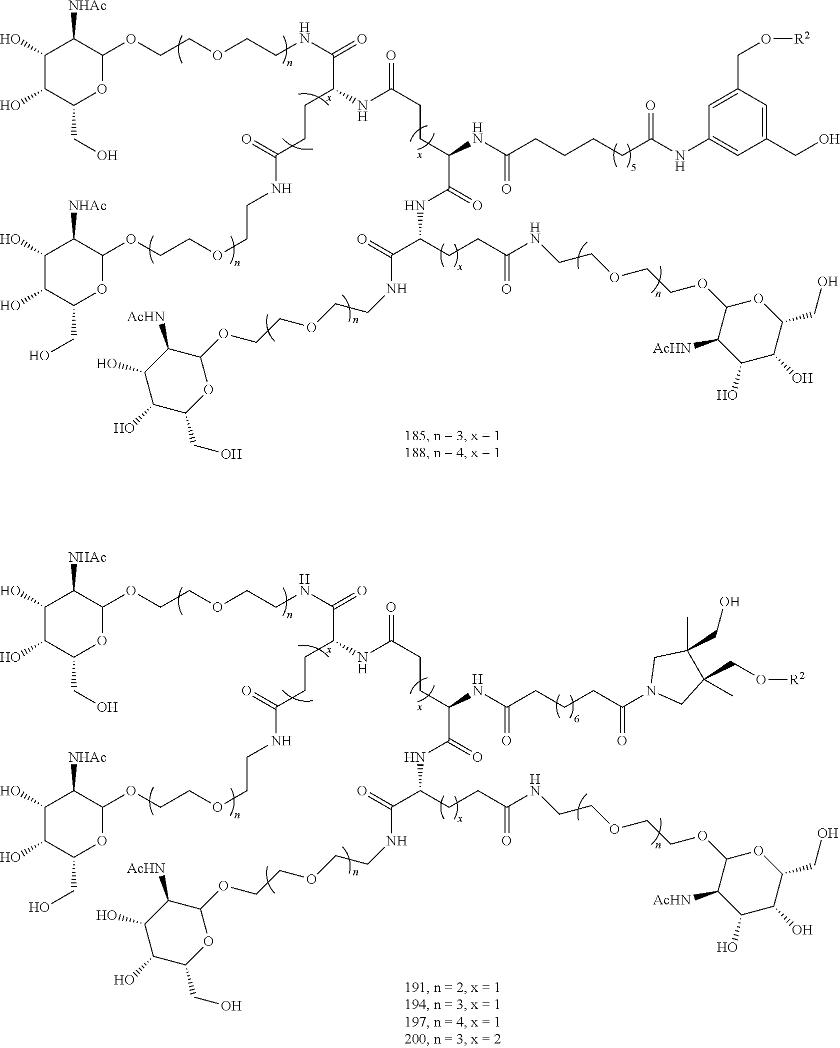

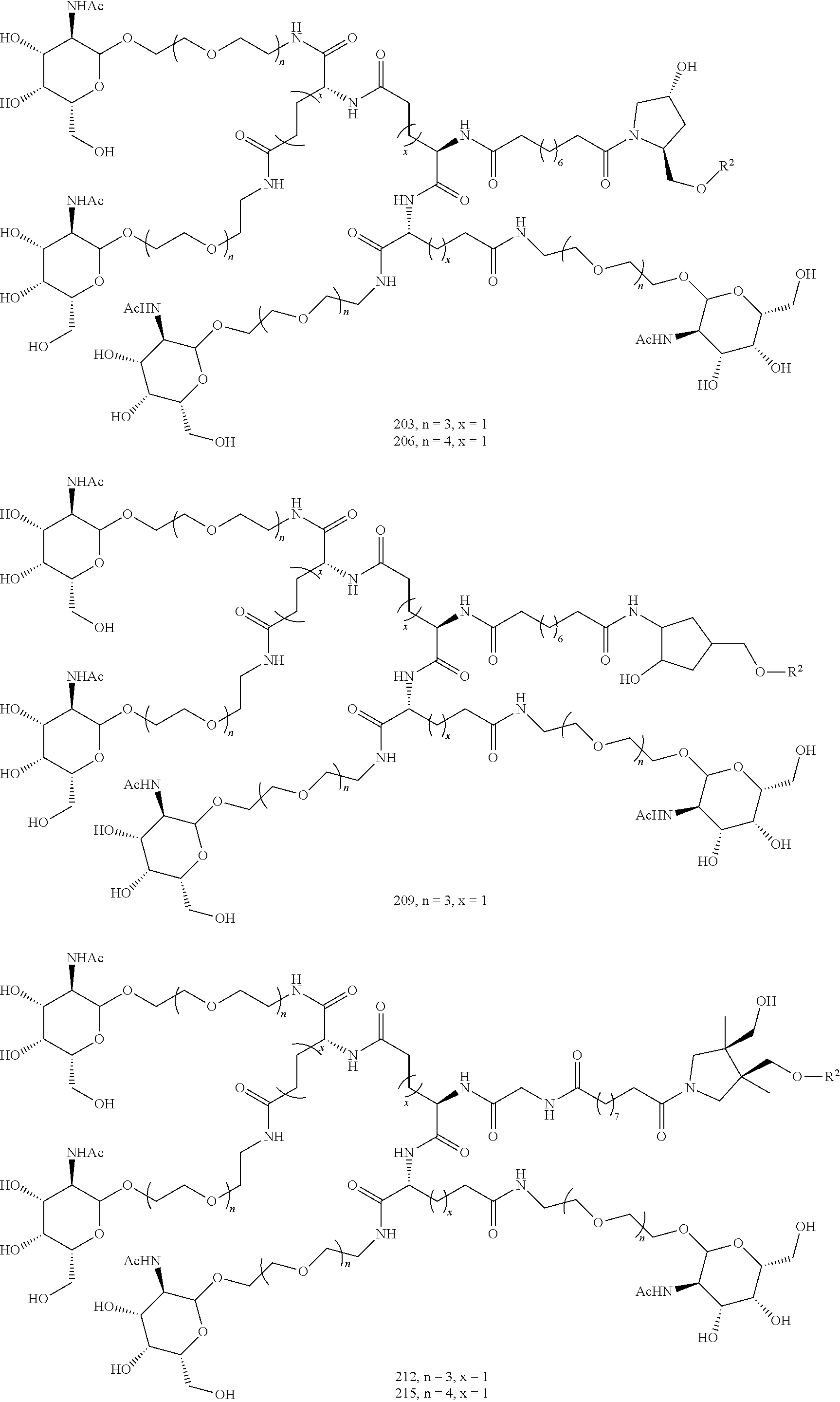

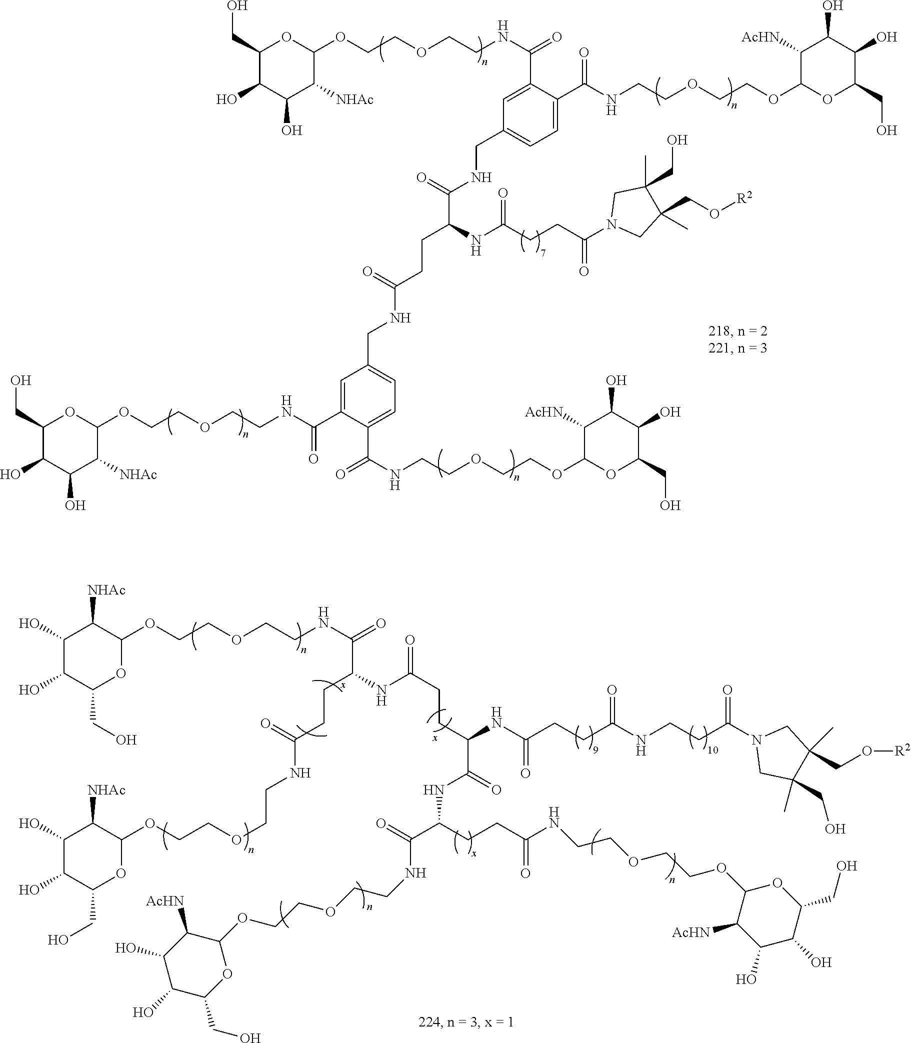

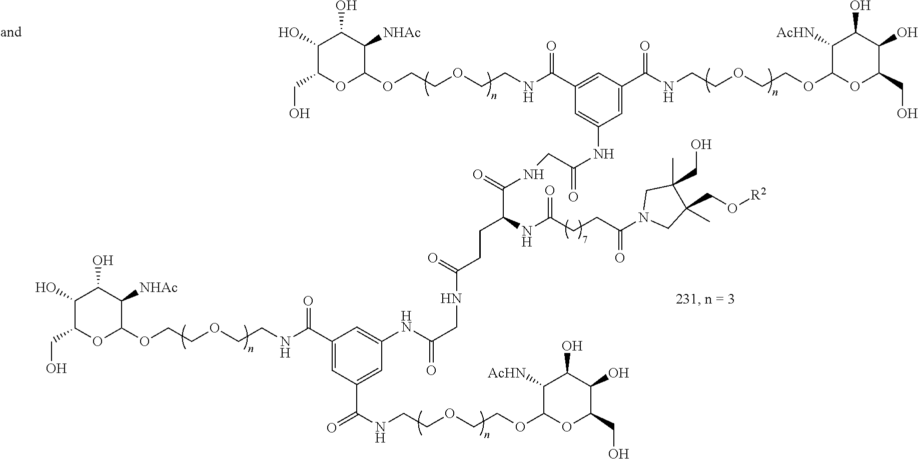

and pharmaceutically acceptable salts thereof.

In certain embodiments, the compound of formula (I) is:

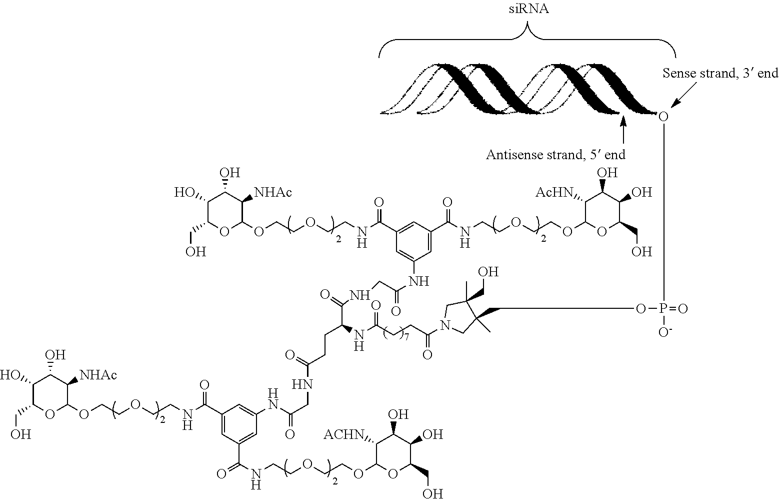

or a pharmaceutically acceptable salt thereof, wherein the siRNA depicted is selected from any one of siRNA 1-siRNA 28.

In certain embodiments, the siRNA is selected from any one of siRNA 1-siRNA 24.

In certain embodiments, the siRNA is selected from any one of siRNA 25-siRNA 28.

In certain embodiments, the siRNA is siRNA 25.

In certain embodiments, the siRNA is siRNA 26.

In certain embodiments, the siRNA is siRNA 27.

In certain embodiments, the siRNA is siRNA 28.

Certain embodiments provide a method for treating acromegaly, comprising administering to a patient in need thereof an effective amount of a compound as described herein.

Certain embodiments provide a method for reducing insulin-like growth factor-1 (IGF-1) in a patient, comprising administering to a patient in need thereof an effective amount of a compound as described herein.

Certain embodiments provide a method for reducing growth hormone in a patient, comprising administering to a patient in need thereof an effective amount of a compound as described herein.

Certain embodiments provide a method for reducing growth hormone receptor (GHR) in the liver in a patient, comprising administering to a patient in need thereof an effective amount of a compound as described herein.

In certain embodiments, the compound of formula (I) is administered subcutaneously.

Certain embodiments provide a double stranded siRNA molecule selected from the group consisting of siRNA 1-siRNA 28.

Certain embodiments provide a composition comprising a double stranded siRNA molecule of claim 19.

As used herein, the following terms have the meanings ascribed to them unless specified otherwise.

Acromegaly

Acromegaly is a hormonal disorder that develops when the pituitary gland produces too much growth hormone. When this happens, bones increase in size, including those of the hands, feet and face. Acromegaly usually affects middle-aged adults, though it can develop at any age. In children who are still growing, too much growth hormone can cause a condition called gigantism. These children have exaggerated bone growth and an abnormal increase in height.

Because acromegaly is uncommon and physical changes occur gradually, the condition sometimes takes a long time to recognize. If it's not treated promptly, acromegaly can lead to serious illness and may even become life-threatening.

A common sign of acromegaly is enlarged hands and feet. People with this disorder often notice that they are not able to put on rings that once fit and that their shoe size has progressively increased. Acromegaly may also cause gradual changes in the shape of your face, such as a protruding lower jaw and brow, an enlarged nose, thickened lips, and wider spacing between your teeth. Because acromegaly tends to progress slowly, early signs may not be obvious for years. Sometimes, people notice the condition only by comparing old photographs with newer ones.

Acromegaly may produce the following signs and symptoms, which can vary from one person to another: enlarged hands and feet, coarsened, enlarged facial features, coarse, oily, thickened skin, excessive sweating and body odor, small outgrowths of skin tissue (skin tags), fatigue and muscle weakness, a deepened, husky voice due to enlarged vocal cords and sinuses, severe snoring due to obstruction of the upper airway, impaired vision, headaches, enlarged tongue, pain and limited joint mobility, menstrual cycle irregularities in women, erectile dysfunction in men, enlarged organs, such as the heart, and loss of interest in sex.

Acromegaly is caused by the pituitary gland overproducing growth hormone (GH) over time. When GH is secreted into your bloodstream, it triggers the liver to produce a hormone called insulin-like growth factor-I (IGF-I). In turn, IGF-I stimulates the growth of bones and other tissues. If the pituitary gland makes too much GH, excessive amounts of IGF-I can result. Too much IGF-I can cause abnormal growth of soft tissues and skeleton and other signs and symptoms characteristic of acromegaly and gigantism.

In adults, a tumor (e.g., a pituitary or nonpituitary tumor) is the most common cause of too much GH production. Most cases of acromegaly are caused by a noncancerous benign tumors (adenomas) of the pituitary gland. The tumor secretes excessive amounts of growth hormone, causing many of the signs and symptoms of acromegaly. Some of the symptoms of acromegaly, such as headaches and impaired vision, are due to the tumor mass pressing on nearby brain tissues. In a few people with acromegaly, tumors in other parts of the body, such as the lungs or pancreas, cause the disorder. Sometimes, these tumors secrete GH. In other cases, the tumors produce a hormone called growth hormone-releasing hormone (GH-RH), which stimulates the pituitary gland to make more GH.

Progression of acromegaly can result in major health problems. Complications may include: high blood pressure (hypertension), cardiovascular disease, particularly enlargement of the heart (cardiomyopathy), osteoarthritis, diabetes mellitus, goiter, precancerous growths (polyps) on the lining of the colon, sleep apnea, carpal tunnel syndrome, spinal cord compression, and vision loss. Early treatment of acromegaly can prevent these complications from developing or becoming worse. Untreated, acromegaly and its complications can lead to premature death.

Current treatments include surgery to attempt to remove the tumor, radiation treatment (e.g., conventional radioation therapy, proton beam therapy or stereotaxic radiosurgery), and medications. Medications used to lower the production or block the action of GH include drugs that reduce excess growth hormone secretion (e.g., somatostatin analogues). The classic standard of care for acromegaly is octreotide (Sandostatin), which is a somatostatin analogue (SSA) that prevents the release of GH from the pituitary gland. Several other SSAs, such as pasireotide (Signifor) and lanreotide (Somatuline) are also commercially available. These SSAs all require regular dosing and there are large segments of the population that are treatment refractory. Further, these SSAs have varying but significant tolerability concerns such as injection site reactions, diarrhea, and bradycardia. The drugs octreotide and lanreotide are synthetic versions of the brain hormone somatostatin. They can interfere with the excessive secretion of GH by the pituitary gland, causing rapid declines in GH levels. These drugs are given by injection into the muscles of the buttocks (gluteal muscles) once a month by a health care professional. Drugs to lower hormone levels (e.g., dopamine agonists) can also be used. The oral medications cabergoline and bromocriptine lower levels of GH and IGF-I in some people. The tumor may decrease in size in some people taking a dopamine agonist. Some people may develop compulsive behaviors, such as gambling, while taking these medications. Drugs to block the action of GH (e.g., growth hormone antagonist) can also be used. The medication pegvisomant blocks the effect of GH on body tissues. Pegvisomant may be particularly helpful for people who haven't had good success with other forms of treatment. Given as a daily injection, this medication can normalize IGF-I levels and relieve symptoms in most people with acromegaly, but it doesn't lower GH levels or reduce the tumor size.

Tables 1 and 2 below provide certain siRNA molecules useful in conjugates and methods described herein. The GalNAc portion of the conjugate is notes as (GalNAc) in Tables 1 and 2. The exemplary GalNAc used is depicted in the Example section.

TABLE 1
Acromegaly siRNA used for in vitro screening

| siRNA | Sense Strand Sequence 5′→3′ | Anti-Sense Strand Sequence 5′→3′ |
|---|---|---|
| 1 | asasgaGfCfUfacguauuuaa-(GalNAc) | usUfsaaauacguagcUfcUfuggsgsa |
| 2 | gsusagCfAfGfugauugucua-(GalNAc) | usAfsgacaaucacugCfuAfcuasasa |
| 3 | csusagAfAfUfugaguguuua-(GalNAc) | usAfsaacacucaauuCfuAfgcususu |
| 4 | uscsucAfGfAfaugucauuua-(GalNAc) | usAfsaaugacauucuGfaGfacusgsa |
| 5 | gsasuaCfUfAfagcauugaaa-(GalNAc) | usUfsucaaugcuuagUfaUfcaasasa |
| 6 | csasuaGfCfAfcaggcuaaua-(GalNAc) | usAfsuuagccugugcUfaUfggususu |
| 7 | usasuaCfCfUfccauucauaa-(GalNAc) | usUfsaugaauggaggUfaUfaguscsu |
| 8 | cscscaAfGfAfgcuacguaua-(GalNAc) | usAfsuacguagcucuUfgGfgaasasc |
| 9 | gscsuaAfCfAfgugaugcuaa-(GalNAc) | usUfsagcaucacuguUfaGfcccsasa |
| 10 | uscsuuGfGfGfuugaauuuaa-(GalNAc) | usUfsaaauucaacccAfaGfaguscsa |
| 11 | uscscaAfGfAfgcuacauaaa-(GalNAc) | usUfsuauguagcucuUfgGfagasasa |
| 12 | asusagCfAfCfaggcuaauua-(GalNAc) | usAfsauuagccugugCfuAfuggsusu |
| 13 | usascuAfAfGfcauugaauga-(GalNAc) | usCfsauucaaugcuuAfgUfaucsasa |
| 14 | ususcaCfUfAfguaugacuaa-(GalNAc) | usUfsagucauacuagUfgAfauasasu |
| 15 | asgsgaAfGfCfaagcuuaaua-(GalNAc) | usAfsuuaagcuugcuUfcCfuaasasa |
| 16 | gscsgaGfAfGfacuuuuucaa-(GalNAc) | usUfsgaaaaagucucUfcGfcucsasg |
| 17 | ususcaUfGfAfuagcuauaaa-(GalNAc) | usUfsuauagcuaucaUfgAfaugsgsc |
| 18 | asgscgAfGfAfgacuuuuuca-(GalNAc) | usGfsaaaaagucucuCfgCfucasgsg |
| 19 | cscsaaGfAfGfcuacguauua-(GalNAc) | usAfsauacguagcucUfuGfggasasa |
| 20 | asascaGfCfCfugacaacaua-(GalNAc) | usAfsuguugucaggcUfgUfugusgsa |
| 21 | cscsauUfAfUfucacuaguaa-(GalNAc) | usUfsacuagugaauaAfuGfgcususa |
| 22 | gscsagUfUfUfauauuuaaca-(GalNAc) | usGfsuuaaauauaaaCfuGfccasgsa |
| 23 | asusuuAfUfCfgcagaccuua-(GalNAc) | usAfsaggucugcgauAfaAfuggsgsa |
| 24 | usasggAfAfGfcaagcuuaaa-(GalNAc) | usUfsuaagcuugcuuCfcUfaaasasa |

s = phosphorothioate
lowercase x = 2′oME modified base
Xf = 2′fluoro modified base
uppercase X = unmodified base

TABLE 2
Acromegaly siRNA used in toxicology and/or non-human primate studies

| siRNA | Sense Strand Sequence 5′→3′ | Anti-Sense Strand Sequence 5′→3′ |
|---|---|---|
| 25 | ususcaUfGfAfuagcuauaaa-(GalNAc) | usUfsuauagcuaucaUfgAfaugsgscU |
| 26 | gscsuaAfCfAfgugaugcuaa-(GalNAc) | usUfsagcaucacuguUfaGfcccsasaU |
| 27 | uscsucAfGfAfaugucauuua-(GalNAc) | usAfsaaugacauucuGfaGfacusgsaU |
| 28 | asasgaGfCfUfacguauuuaa-(GalNAc) | usUfsaaauacguagcUfcUfuggsgsaU |

s = phosphorothioate
lowercase x = 2′oME modified base
Xf = 2′fluoro modified base
uppercase X = unmodified base

In certain embodiments, the siRNA is siRNA 1. In certain embodiments, the siRNA is siRNA 2. In certain embodiments, the siRNA is siRNA 3. In certain embodiments, the siRNA is siRNA 4. In certain embodiments, the siRNA is siRNA 5. In certain embodiments, the siRNA is siRNA 6. In certain embodiments, the siRNA is siRNA 7. In certain embodiments, the siRNA is siRNA 8. In certain embodiments, the siRNA is siRNA 9. In certain embodiments, the siRNA is siRNA 10. In certain embodiments, the siRNA is siRNA 11. In certain embodiments, the siRNA is siRNA 12. In certain embodiments, the siRNA is siRNA 13. In certain embodiments, the siRNA is siRNA 14. In certain embodiments, the siRNA is siRNA 15. In certain embodiments, the siRNA is siRNA 16. In certain embodiments, the siRNA is siRNA 17. In certain embodiments, the siRNA is siRNA 18. In certain embodiments, the siRNA is siRNA 19. In certain embodiments, the siRNA is siRNA 20. In certain embodiments, the siRNA is siRNA 21. In certain embodiments, the siRNA is siRNA 22. In certain embodiments, the siRNA is siRNA 23. In certain embodiments, the siRNA is siRNA 24. In certain embodiments, the siRNA is siRNA 25. In certain embodiments, the siRNA is siRNA 26. In certain embodiments, the siRNA is siRNA 27. In certain embodiments, the siRNA is siRNA 28.

The siRNA molecules and conjugates described herein can be used, in certain embodiments, in combination with surgical treatment, radiation treatment (e.g., conventional radioation therapy, proton beam therapy or stereotaxic radiosurgery), and/or other medications.

The term “conjugate” as used herein includes compounds of formula (I) that comprise an oligonucleotide (e.g., an siRNA molecule) linked to a targeting ligand. Thus, the terms compound and conjugate may be used herein interchangeably.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-4 (e.g., 1, 2, 3, or 4) modified and/or unmodified deoxythymidine (t or dT) nucleotides, 1-4 (e.g., 1, 2, 3, or 4) modified (e.g., 2′OMe) and/or unmodified uridine (U) ribonucleotides, and/or 1-4 (e.g., 1, 2, 3, or 4) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion Tech Notes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP) or O-benzotriazol-1-yl-N,N,N′,N′-tetramethyluronium hexafluorophosphate (HBTU).

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Accordingly, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane (including straight and branched alkanes), as exemplified by —CH₂CH₂CH₂CH₂— and —CH(CH₃)CH₂CH₂—.

The term “cycloalkyl,” “carbocyclic,” or “carbocycle” refers to hydrocarbon ring system having 3 to 20 overall number of ring atoms (e.g., 3-20 membered cycloalkyl is a cycloalkyl with 3 to 20 ring atoms, or C₃₋₂₀ cycloalkyl is a cycloalkyl with 3-20 carbon ring atoms) and for a 3-5 membered cycloalkyl being fully saturated or having no more than one double bond between ring vertices and for a 6 membered cycloalkyl or larger being fully saturated or having no more than two double bonds between ring vertices. As used herein, “cycloalkyl,” “carbocyclic,” or “carbocycle” is also meant to refer to bicyclic, polycyclic and spirocyclic hydrocarbon ring system, such as, for example, bicyclo[2.2.1]heptane, pinane, bicyclo[2.2.2]octane, adamantane, norbornene, spirocyclic C₅₋₁₂ alkane, etc. As used herein, the terms, “alkenyl,” “alkynyl,” “cycloalkyl,”, “carbocycle,” and “carbocyclic,” are meant to include mono and polyhalogenated variants thereof.

The term “heterocycloalkyl,” “heterocyclic,” or “heterocycle” refers to a saturated or partially unsaturated ring system radical having the overall having from 3-20 ring atoms (e.g., 3-20 membered heterocycloalkyl is a heterocycloalkyl radical with 3-20 ring atoms, a C₂₋₁₉ heterocycloalkyl is a heterocycloalkyl having 3-10 ring atoms with between 2-19 ring atoms being carbon) that contain from one to ten heteroatoms selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycyclic ring system. Non limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The terms “halo” or “halogen,” by themselves or as part of another substituent, mean, unless otherwise stated, a fluorine, chlorine, bromine, or iodine atom. The term “(halo)alkyl” is meant to include both a “alkyl” and “haloalkyl” substituent. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, difluoromethyl, and the like.

The term “aryl” means a carbocyclic aromatic group having 6-14 carbon atoms, whether or not fused to one or more groups. Examples of aryl groups include phenyl, naphthyl, biphenyl and the like unless otherwise stated.

The term “heteroaryl” refers to aryl ring(s) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

The term saccharide includes monosaccharides, disaccharides and trisaccharides. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of a compound of formula I through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound of formula I through an ether bond. In one embodiment the term saccharide includes a group of the formula:

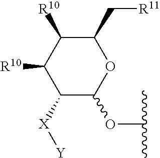

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy. In another embodiment the saccharide can be selected from the group consisting of:

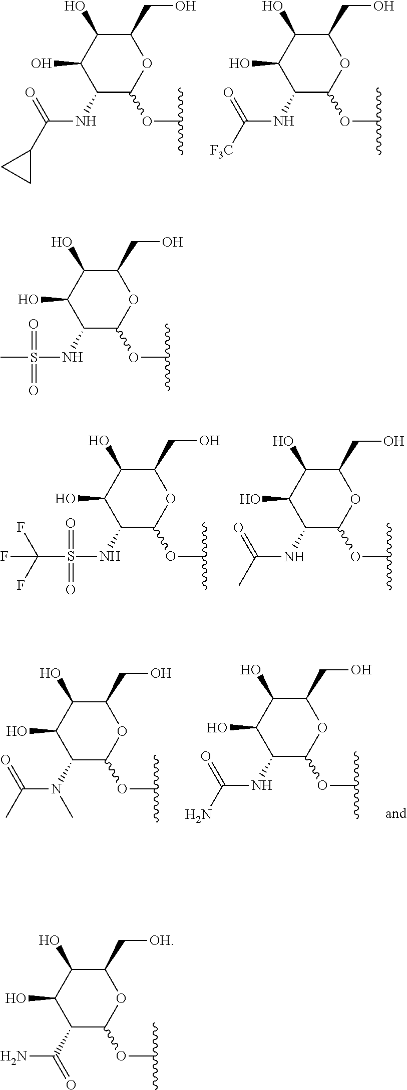

In another embodiment the saccharide can be:

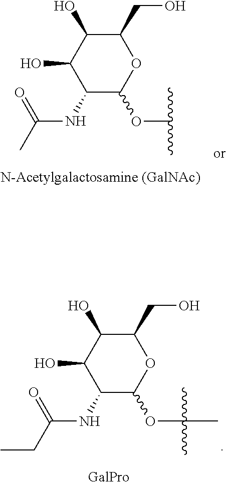 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

The term “animal” includes mammalian species, such as a human, mouse, rat, dog, cat, hamster, guinea pig, rabbit, livestock, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “fusogenic” refers to the ability of a lipid particle, such as a SNALP, to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles such as SNALP means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as an siRNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent such as an siRNA directly to a target site within an organism. For example, an agent can be locally delivered by direct injection into a disease site, other target site, or a target organ such as the liver, heart, pancreas, kidney, and the like.

It will be appreciated by those skilled in the art that compounds having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. Unless otherwise specifically noted, when a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Unless stated otherwise herein, the term “about”, when used in connection with a value or range of values, means plus or minus 5% of the stated value or range of values.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Embodiments

Another aspect provides a composition comprising a double stranded siRNA molecule described herein.

In one embodiment, the composition is a pharmaceutical composition that comprises a pharmaceutically acceptable carrier.

One aspect is a compound of formula I, as set forth about herein, or a salt thereof.

In one embodiment of the compound of formula I, IV a is targeting ligand;

L¹ is absent or a linking group;

L² is absent or a linking group;

R² is a double stranded siRNA molecule selected from the double stranded siRNA of Table 1 and Table 2;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂alkyl-OR^{B} and C₁₋₈ alkyl that is optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10.

In one embodiment R¹ is —C(H)₍₃₋ₚ₎(L³-saccharide)ₚ, wherein each L³ is independently a linking group; p is 1, 2, or 3; and saccharide is a monosaccharide or disaccharide.

In one embodiment the saccharide is:

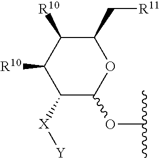

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy,

or a salt thereof.

In one embodiment the saccharide is selected from the group consisting of:

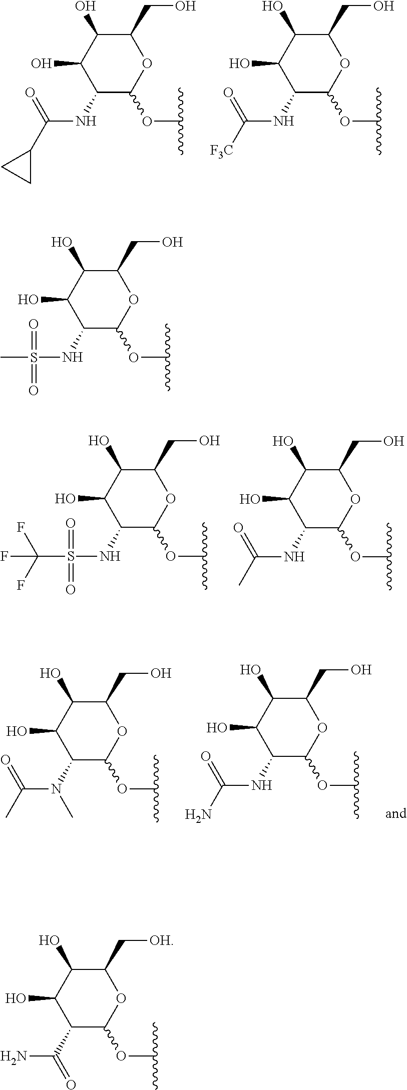

and salts thereof.

In one embodiment the saccharide is:

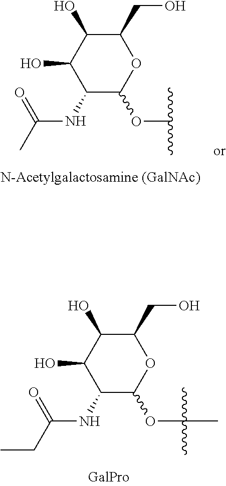 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L³ is:

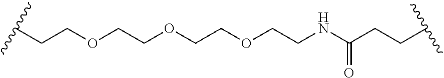 [SMILES: *CCOCCOCCOCCNC(=O)CC*]

or a salt thereof.

In one embodiment R¹ is:

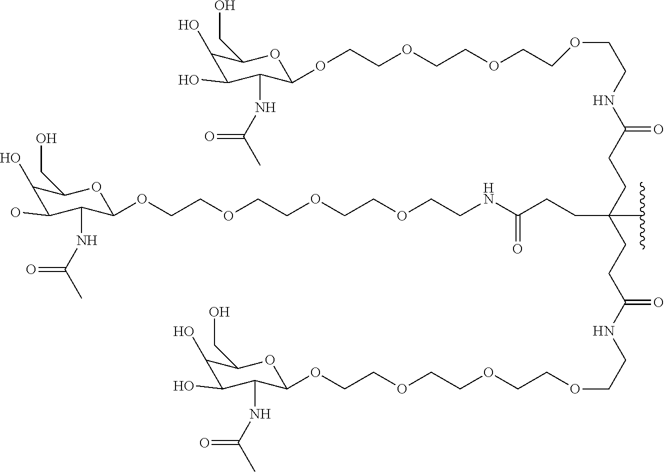 [SMILES: *C(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

or a salt thereof.

In one embodiment R¹ is:

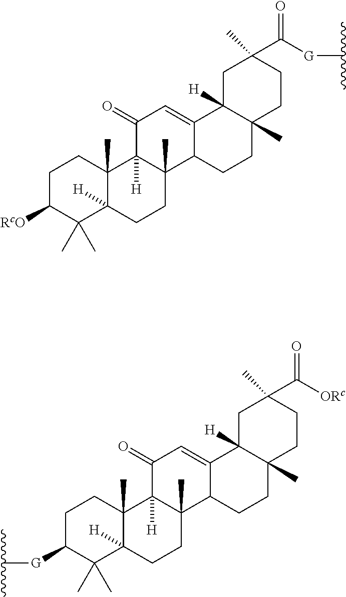

wherein G is —NH— or —O—;

R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, aryl, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy;

or a salt thereof.

In one embodiment R^{C} is:

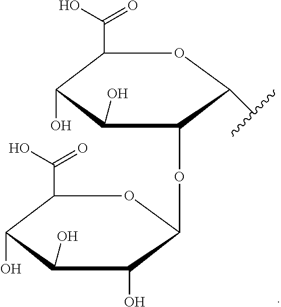

In one embodiment R¹ is:

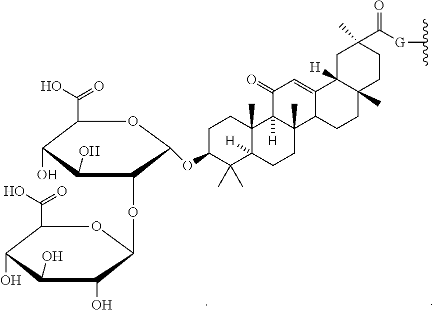

In one embodiment R^{C} is:

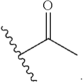 [SMILES: *C(C)=O]

In one embodiment G is —NH—.

In one embodiment R¹ is:

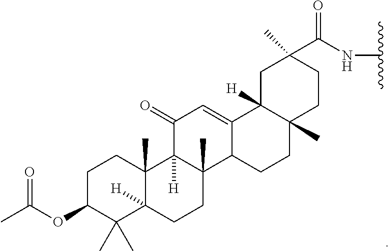

In one embodiment R¹ is:

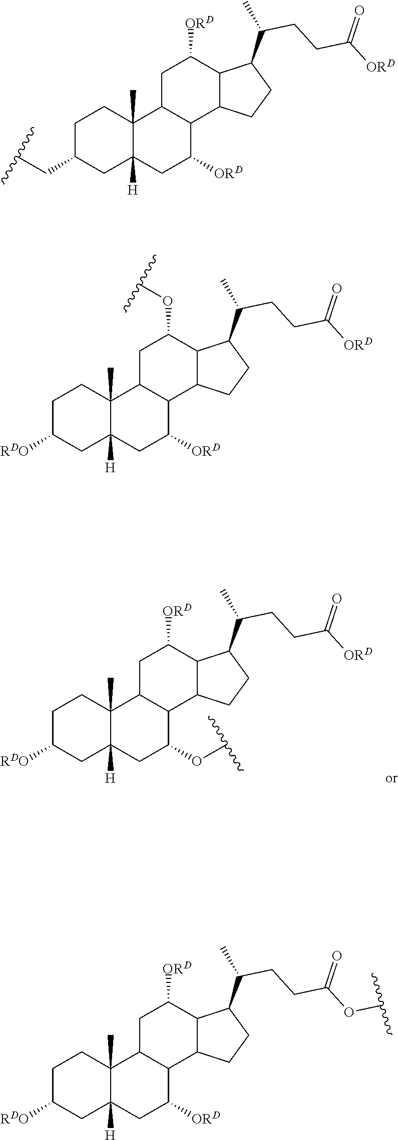

wherein each R^{D} is independently selected from the group consisting of hydrogen, (C₁-C₆)alkyl, (C₉-C₂₀)alkyl silyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and

each R^{W} is independently selected from the group consisting of (C₁-C₄)alkyl and aryl.

In one embodiment linking groups L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In one embodiment L² is connected to R² through —O—.

In one embodiment L¹ is selected from the group consisting of:

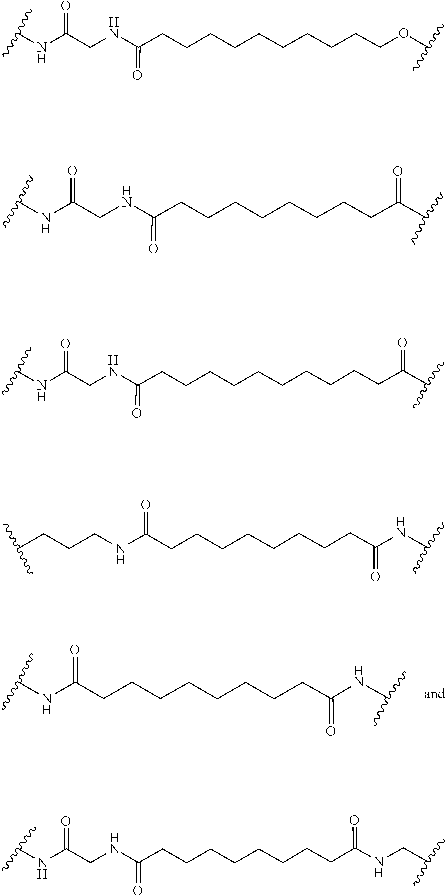 [SMILES: *NC(=O)CNC(=O)CCCCCCCCCCO* ; *NC(=O)CNC(=O)CCCCCCCCC(*)=O ; *NC(=O)CNC(=O)CCCCCCCCCCC(*)=O ; *CCCNC(=O)CCCCCCCCC(=O)N* ; *NC(=O)CCCCCCCCC(=O)N* ; *CNC(=O)CCCCCCCCC(=O)NCC(=O)N*]

In one embodiment L¹ is selected from the group consisting of:

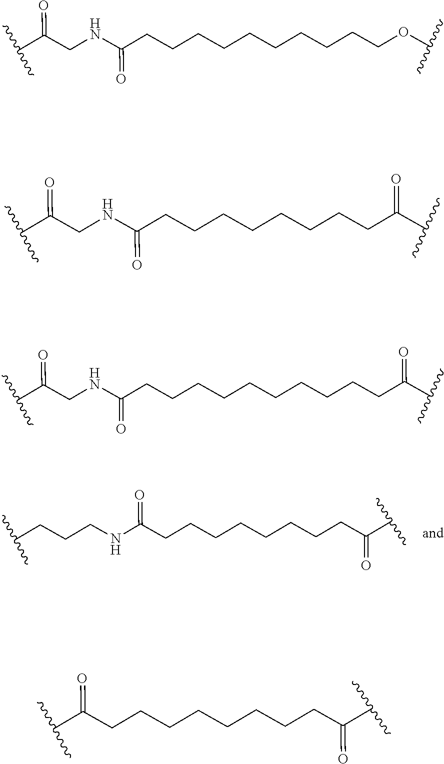 [SMILES: *OCCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O]

and salts thereof.

In one embodiment L² is —CH₂—O— or —CH₂—CH₂—O—.

In one embodiment a compound of formula I has the following formula Ia:

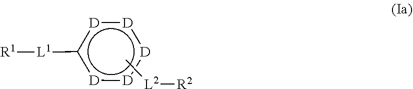

wherein:

each D is independently selected from the group consisting of

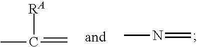

or a salt thereof.

In one embodiment a compound of formula Ia is selected from the group consisting of:

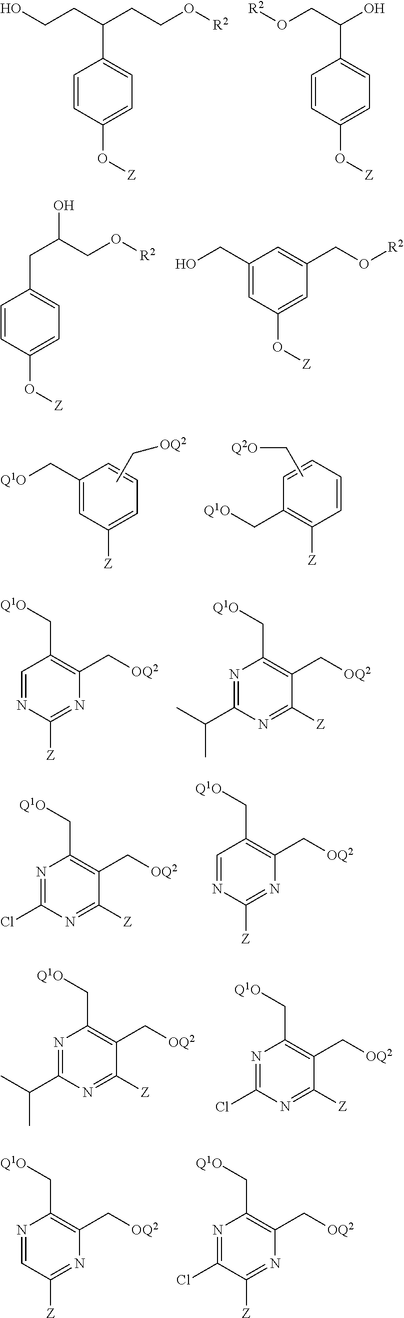

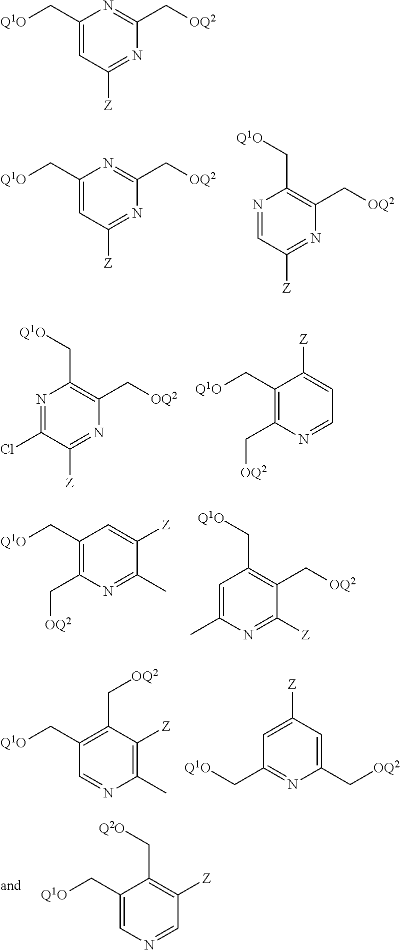

wherein:

Q¹ is hydrogen and Q² is R²; or Q¹ is R² and Q² is hydrogen;

Z is -L¹-R¹;

and salts thereof.

In one embodiment a compound of formula I has the following formula Ib:

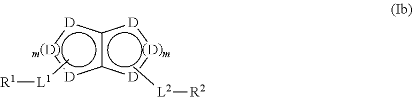

wherein:

each D is independently selected from the group consisting of

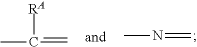

each m is independently 1 or 2; or a salt thereof.

In one embodiment a compound of formula Ib is selected from the group consisting of:

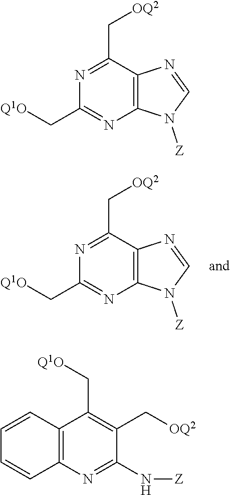

wherein:

Q¹ is hydrogen and Q² is R²; or Q¹ is R² and Q² is hydrogen;

Z is -L¹-R¹;

and salts thereof.

In one embodiment a compound of formula I has the following formula (Ic):

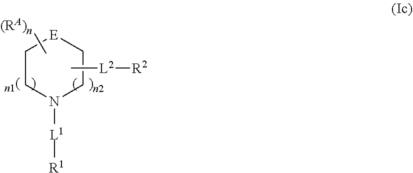

wherein E is —O— or —CH₂—;

n is selected from the group consisting of 0, 1, 2, 3, and 4; and

n1 and n2 are each independently selected from the group consisting of 0, 1, 2, and 3;

or a salt thereof.

In certain embodiments a compound of formula (Ic) is selected from the group consisting of:

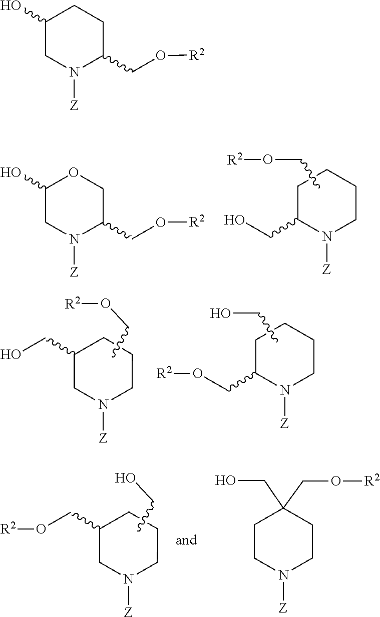

wherein Z is -L¹-R¹;

and salts thereof.

In one embodiment the -A-L²-R² moiety is:

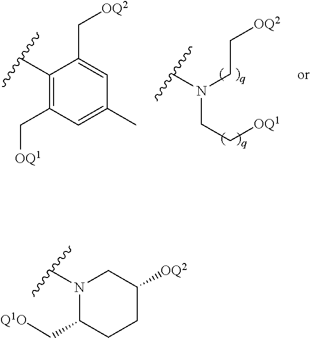

wherein:

Q¹ is hydrogen and Q² is R²; or Q¹ is R² and Q² is hydrogen; and

each q is independently 0, 1, 2, 3, 4 or 5;

or a salt thereof.

In one embodiment a compound of formula (I) is selected from the group consisting of:

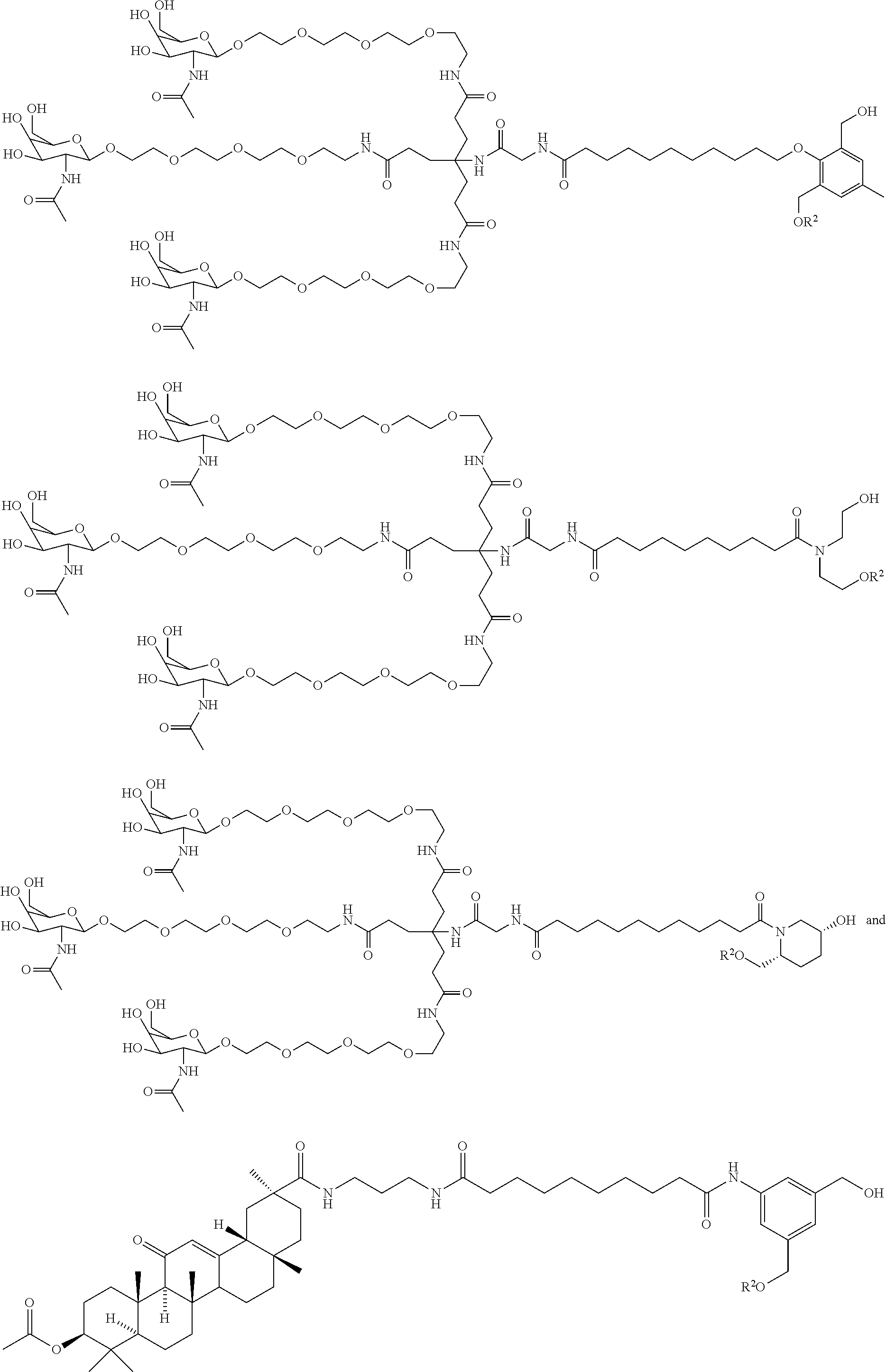

and salts thereof.

In one embodiment R¹ is selected from the group consisting of:

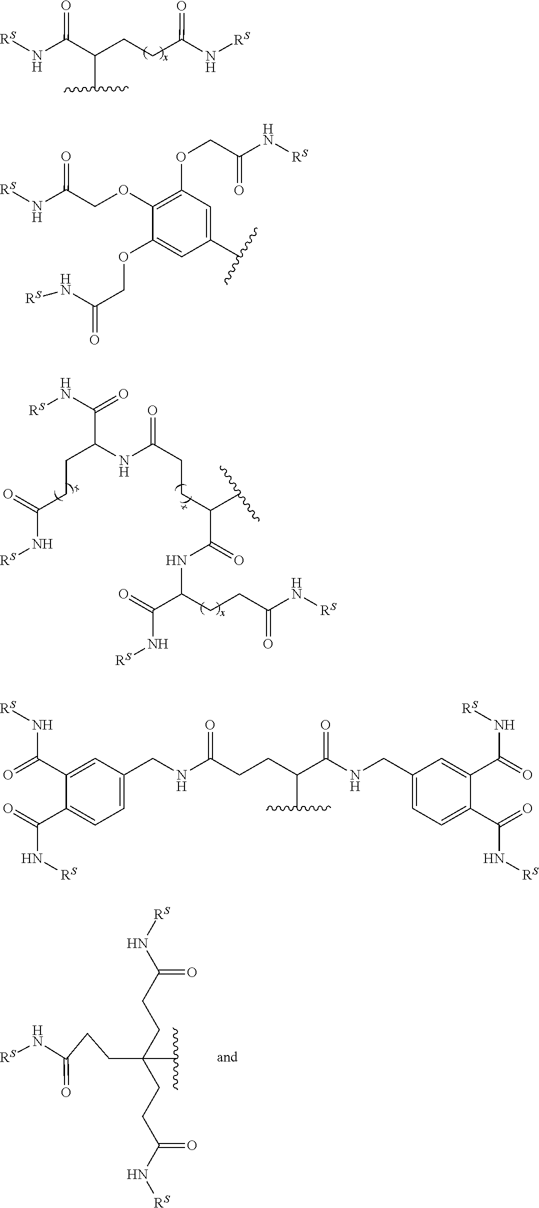

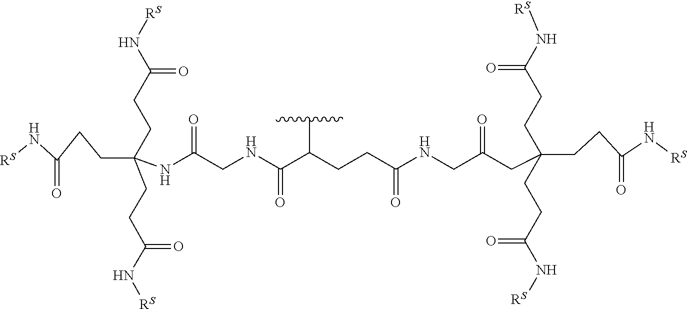

wherein R^{S} is

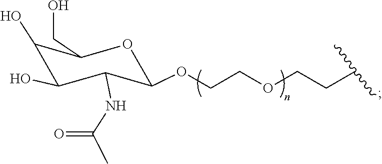

n is 2, 3, or 4;

x is 1 or 2.

In one embodiment L¹ is selected from the group consisting of:

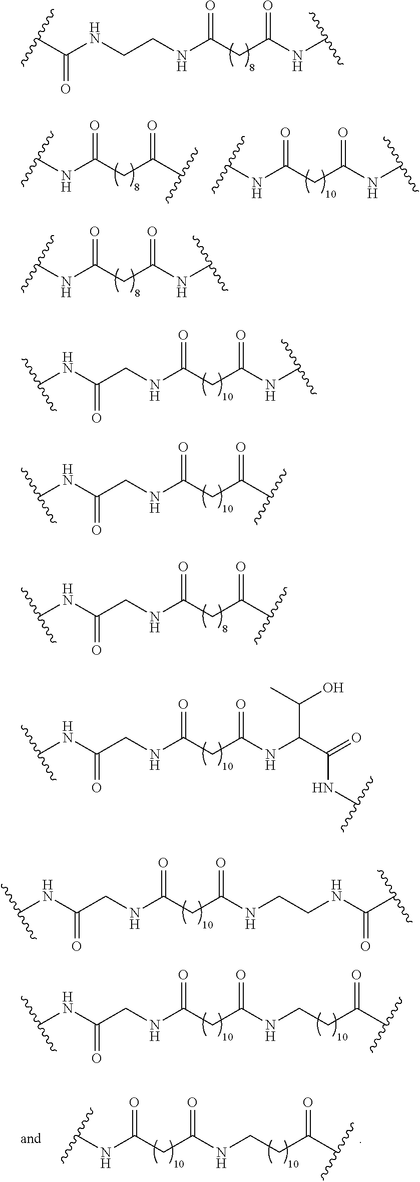

In one embodiment L¹ is selected from the group consisting of:

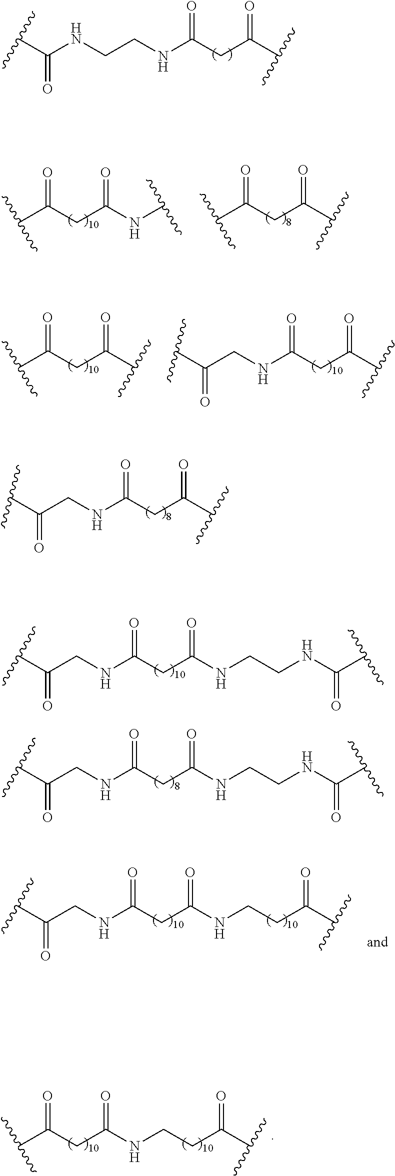

In one embodiment A is absent, phenyl, pyrrolidinyl, or cyclopentyl.

In one embodiment L² is C₁₋₄ alkylene-O— that is optionally substituted with hydroxy.

In one embodiment L² is —CH₂O—, —CH₂CH₂O—, or —CH(OH)CH₂O—.

In one embodiment each R^{A} is independently hydroxy or C₁₋₈ alkyl that is optionally substituted with hydroxyl.

In one embodiment each R^{A} is independently selected from the group consisting of hydroxy, methyl and —CH₂OH.

In one embodiment a compound of formula I has the following formula (Ig):

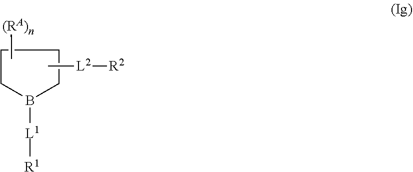

wherein B is —N— or —CH—;

L¹ is absent or —NH—;

L² is C₁₋₄ alkylene-O— that is optionally substituted with hydroxyl or halo;

n is 0, 1, or 2;

or a salt thereof.

In one embodiment a compound of formula I has the following formula (Ig):

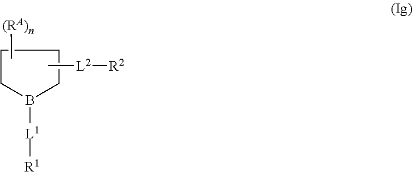

wherein B is —N— or —CH—;

L¹ is absent or —NH—;

L² is C₁₋₄ alkylene-O— that is optionally substituted with hydroxyl or halo;

n is 0, 1, 2, 3, 4, 5, 6, or 7;

or a salt thereof.

In one embodiment a compound of formula I has the following formula (Ig):

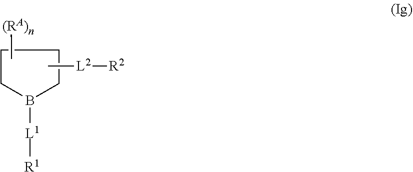

wherein B is —N— or —CH—;

L¹ is absent or —NH—;

L² is C₁₋₄ alkylene-O— that is optionally substituted with hydroxyl or halo;

n is 0, 1, 2, 3, or 4;

or a salt thereof.

In one embodiment a compound of formula Ig is selected from the group consisting of:

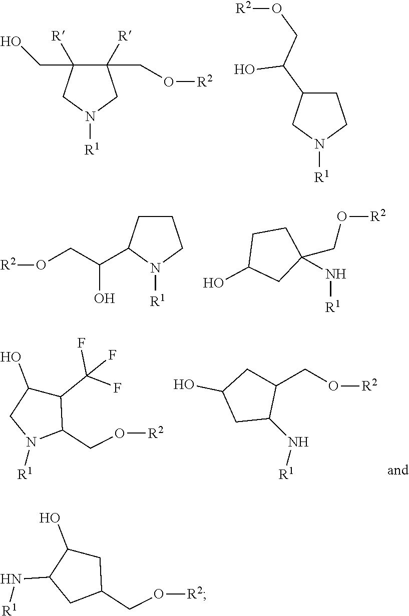

wherein R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

and salts thereof.

In one embodiment a compound of formula I is selected from the group consisting of:

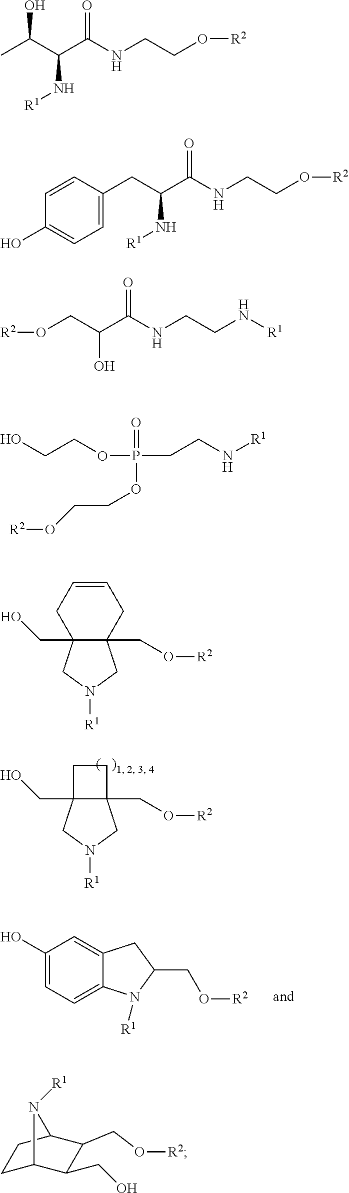

and salts thereof.

In one embodiment the compound of formula I or the salt thereof is selected from the group consisting of:

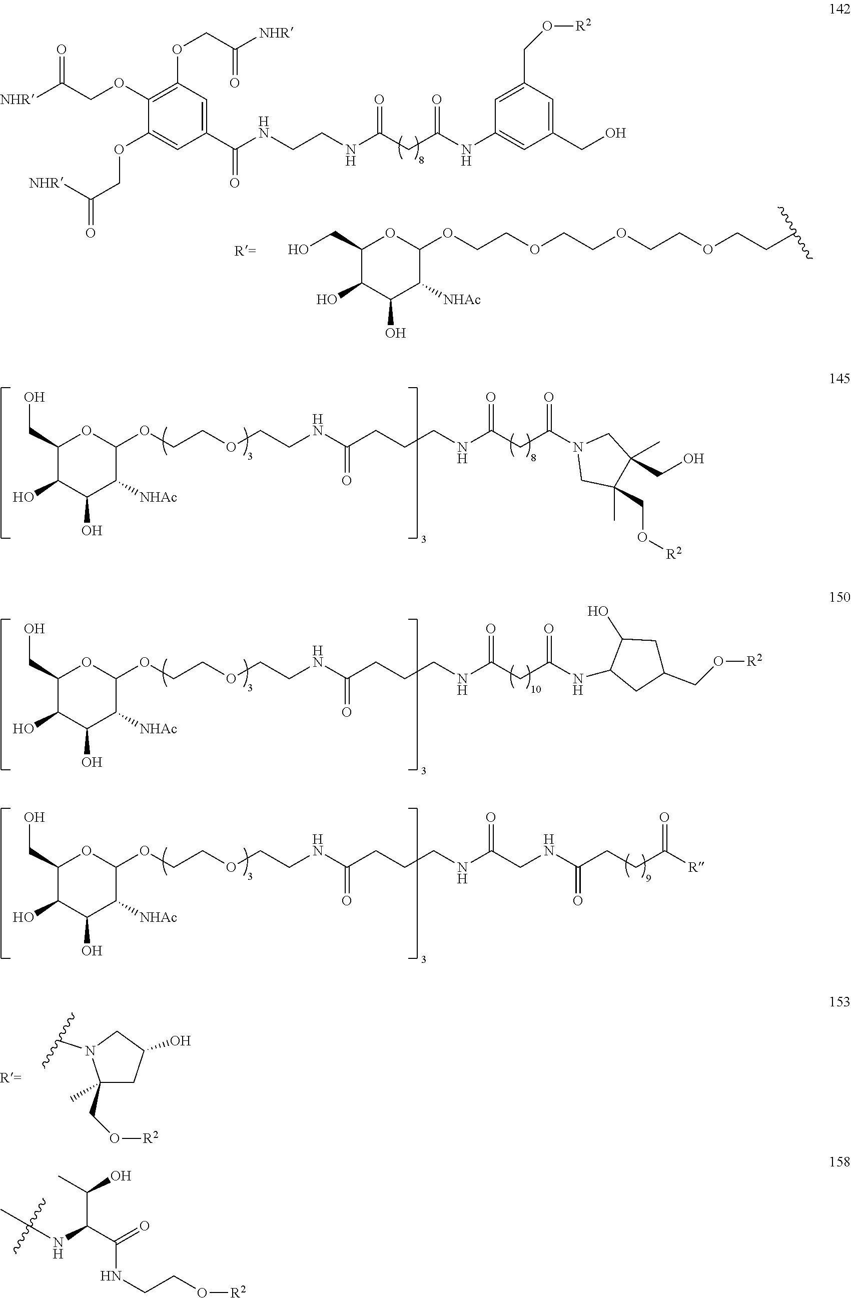

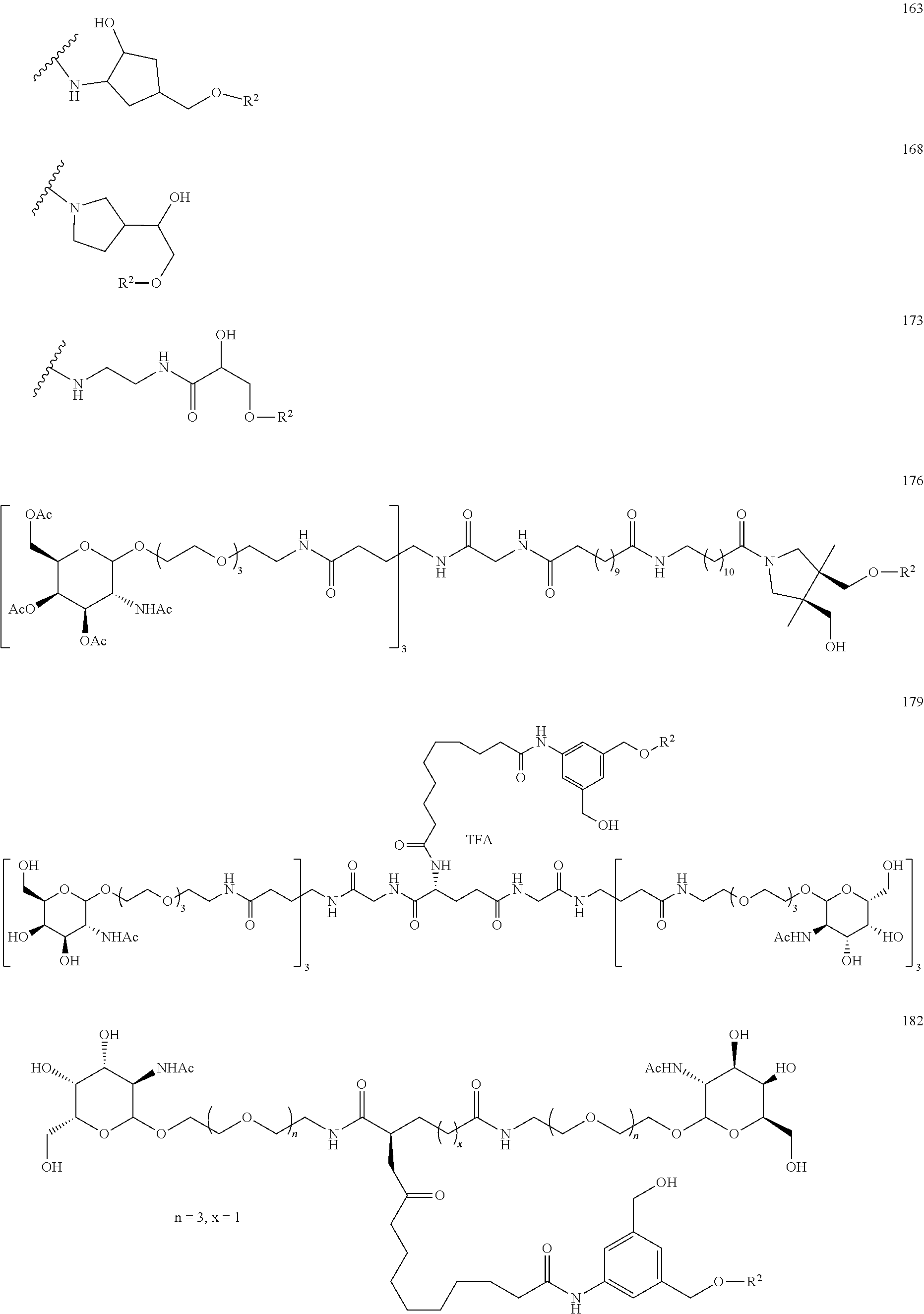

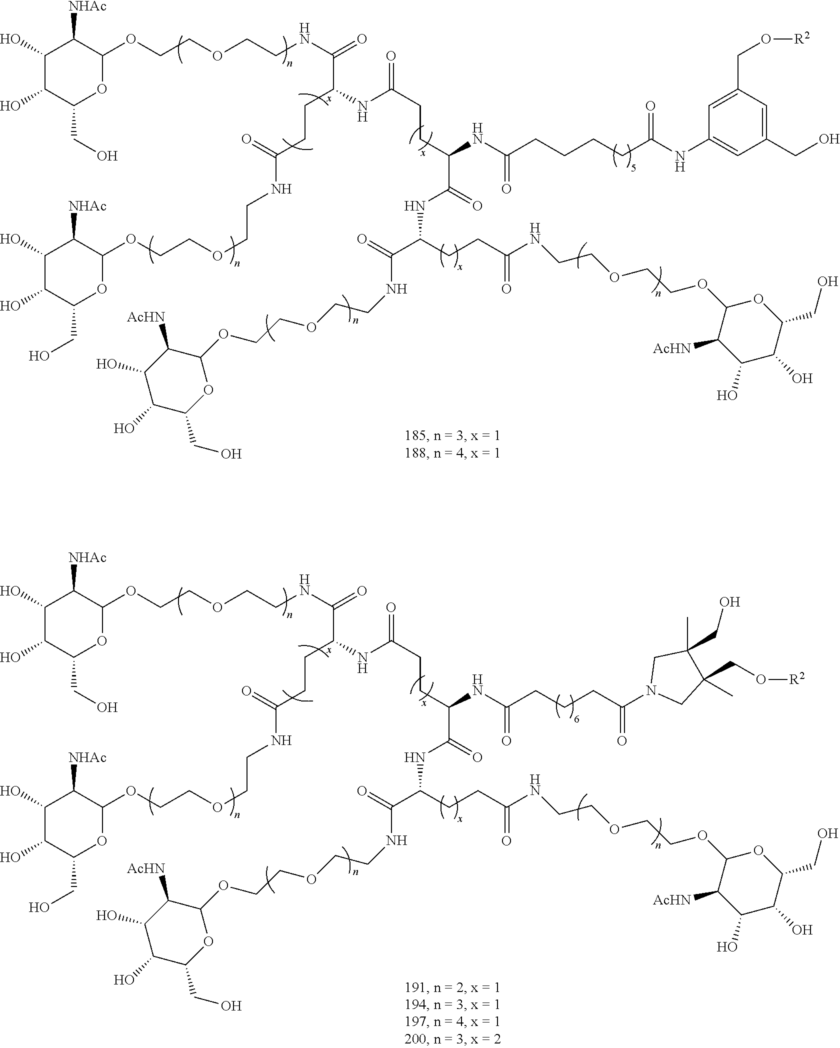

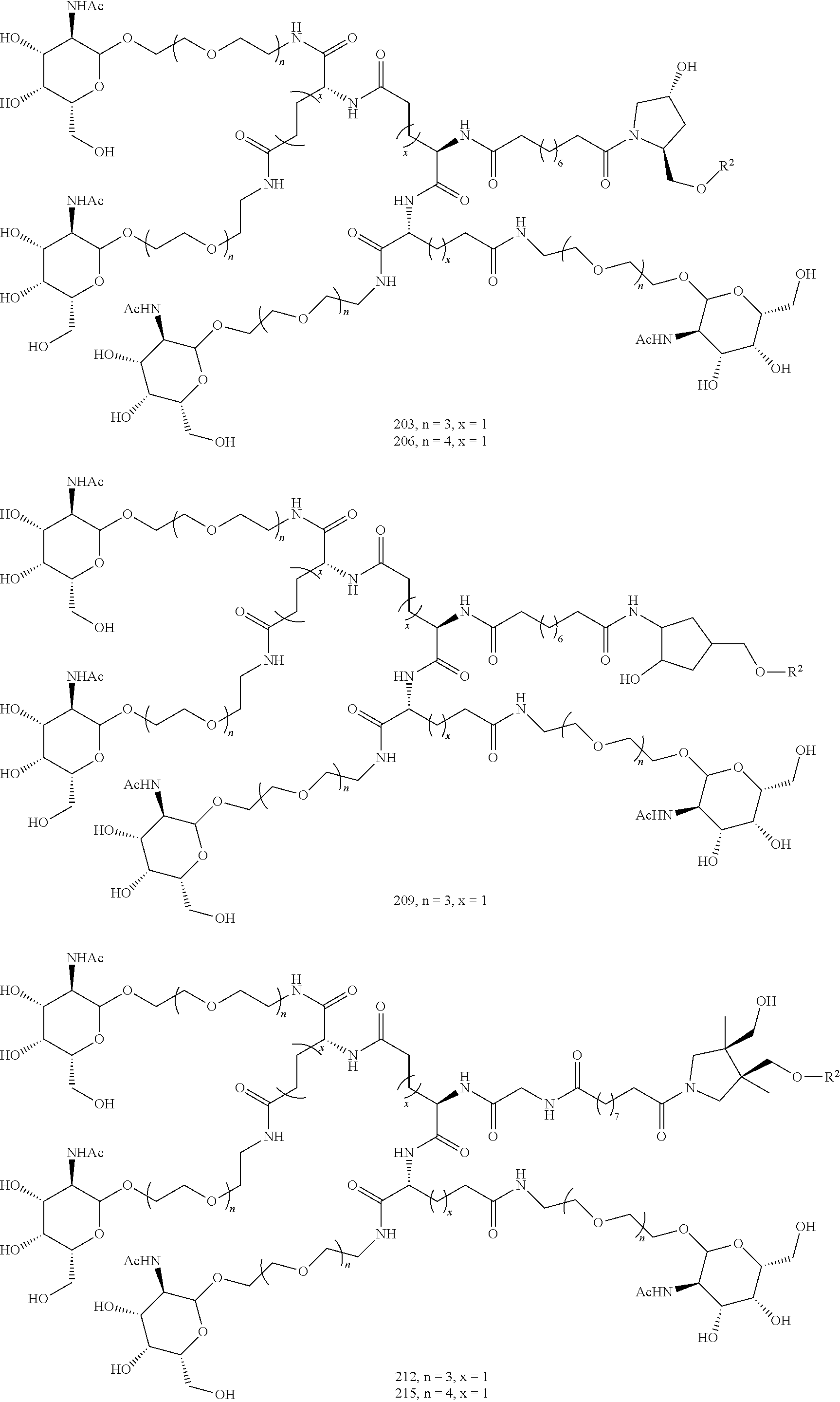

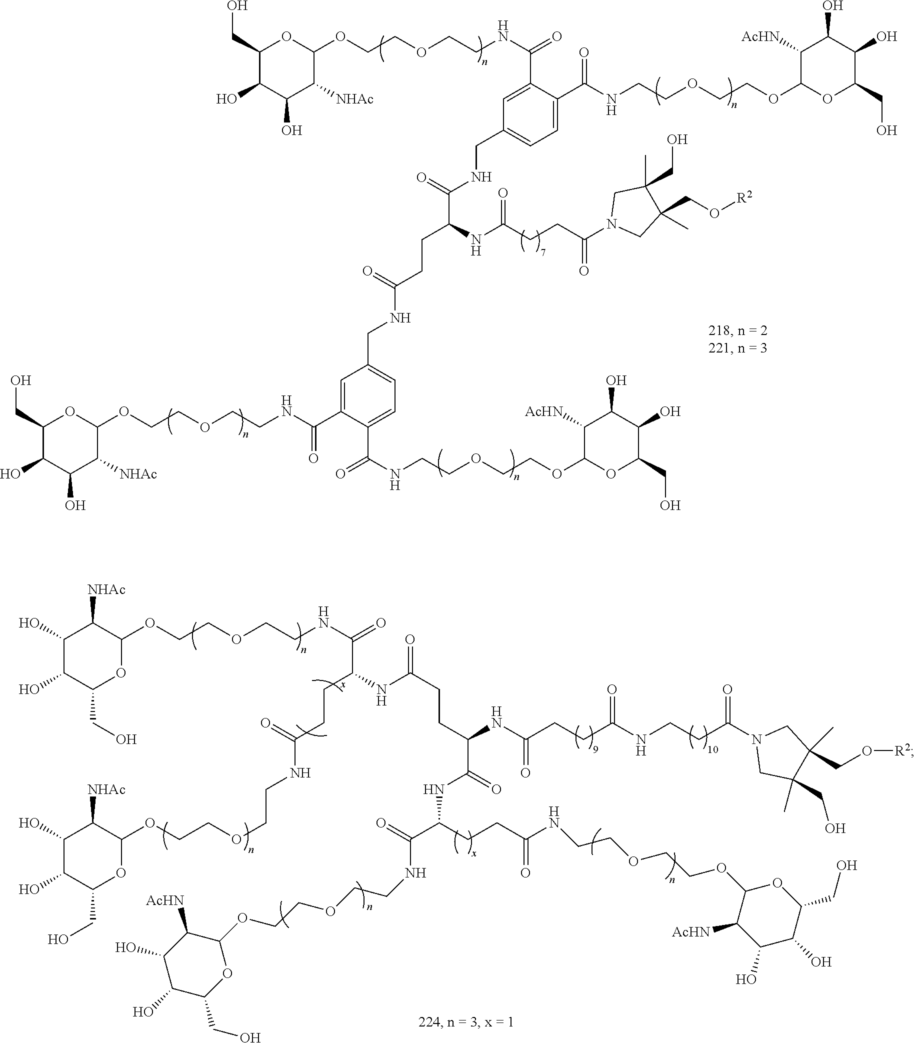

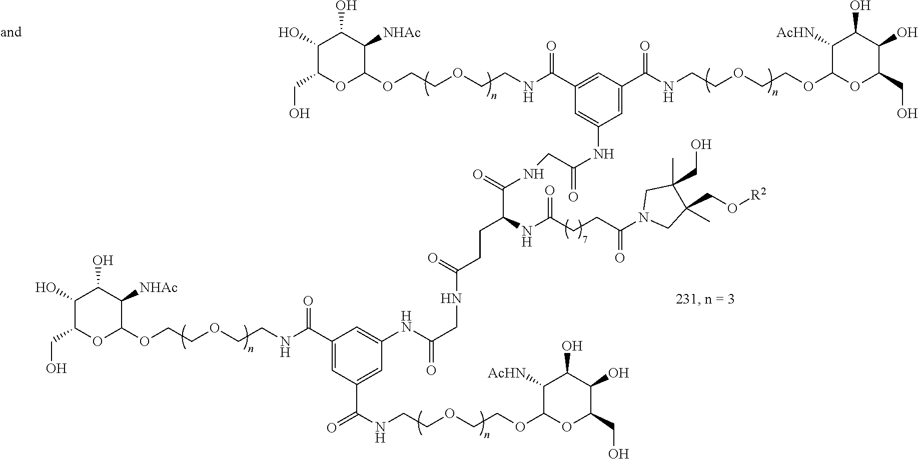

In one embodiment the compound of formula I or the salt thereof is selected from the group consisting of:

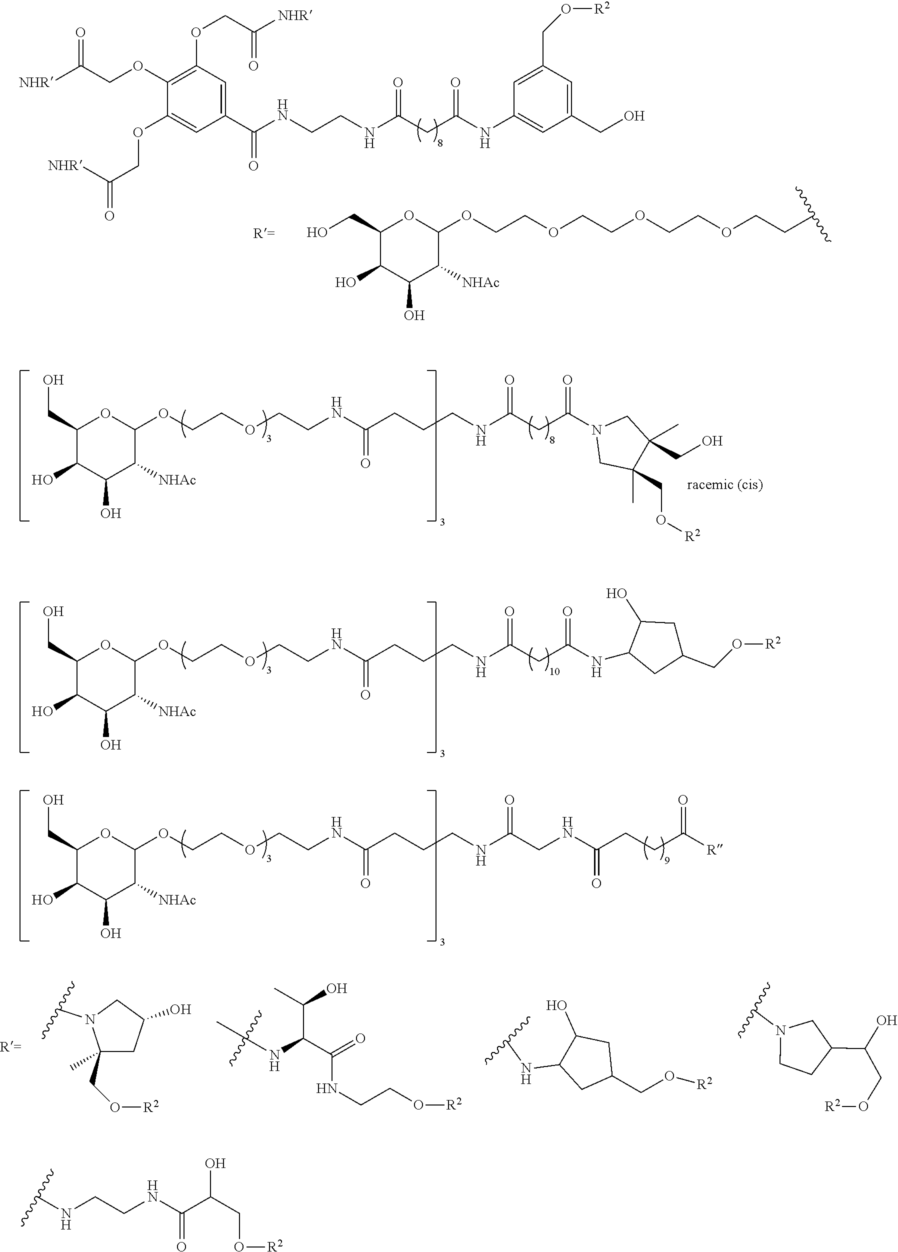

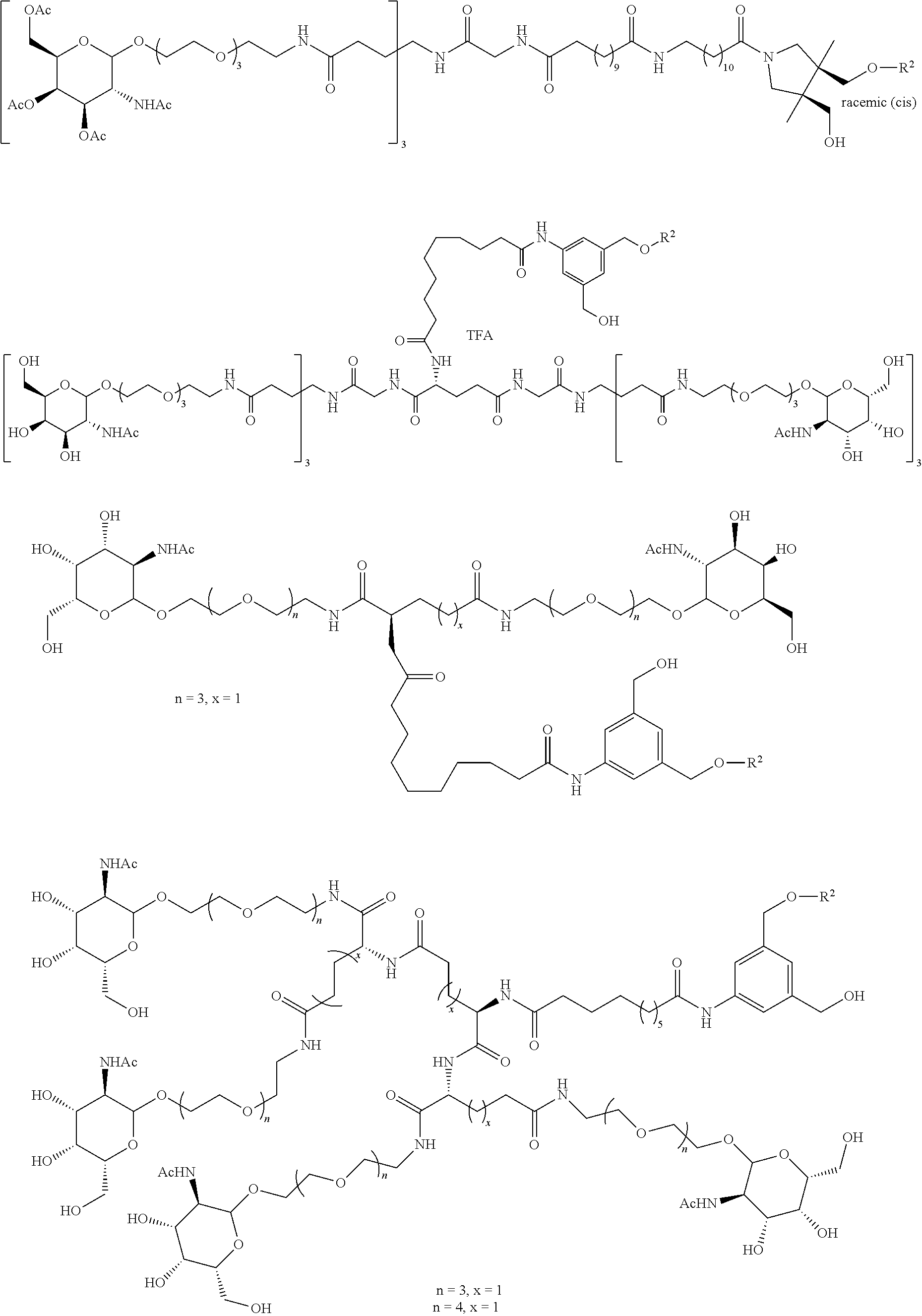

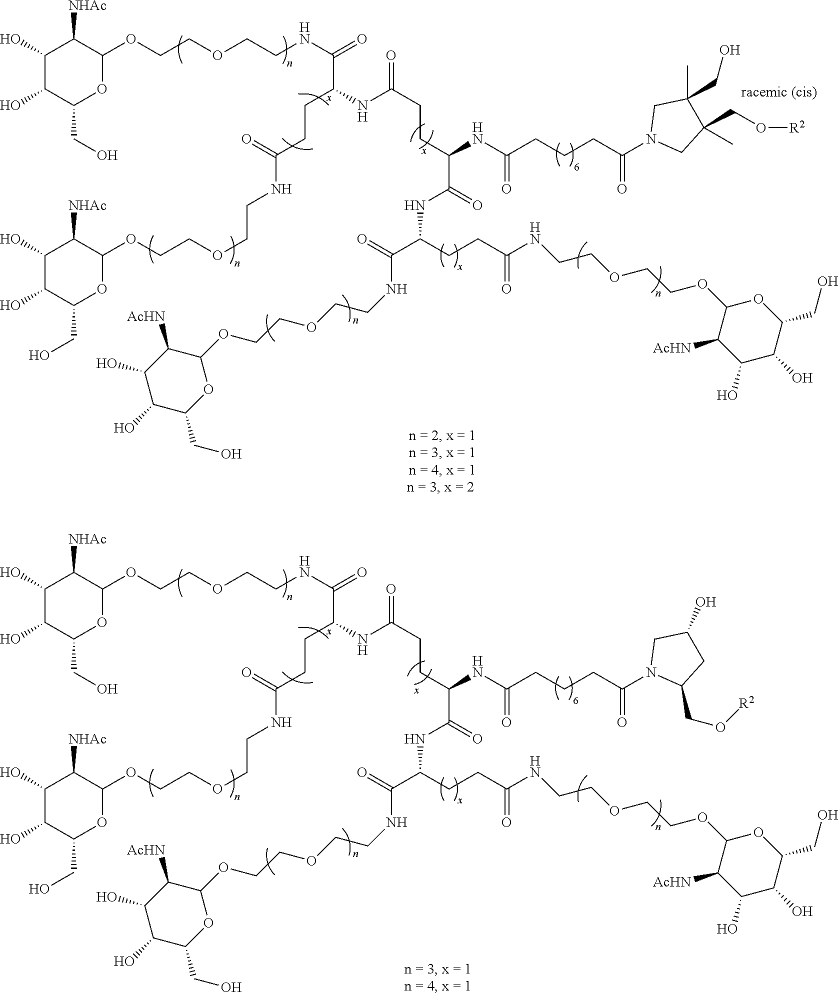

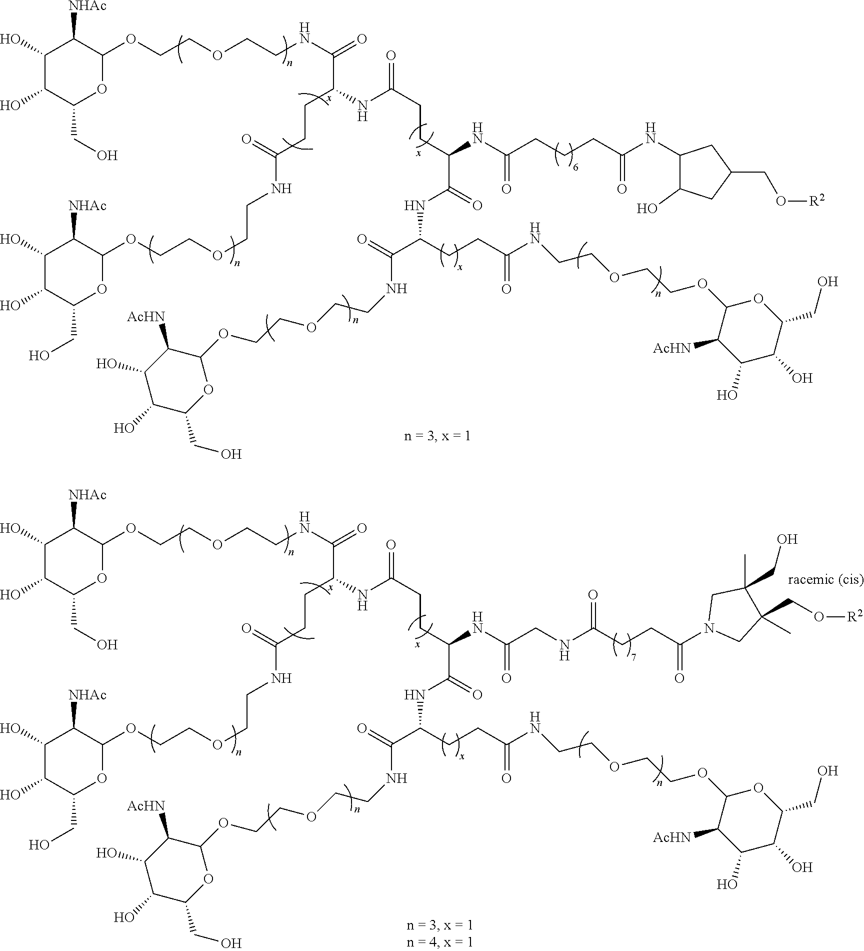

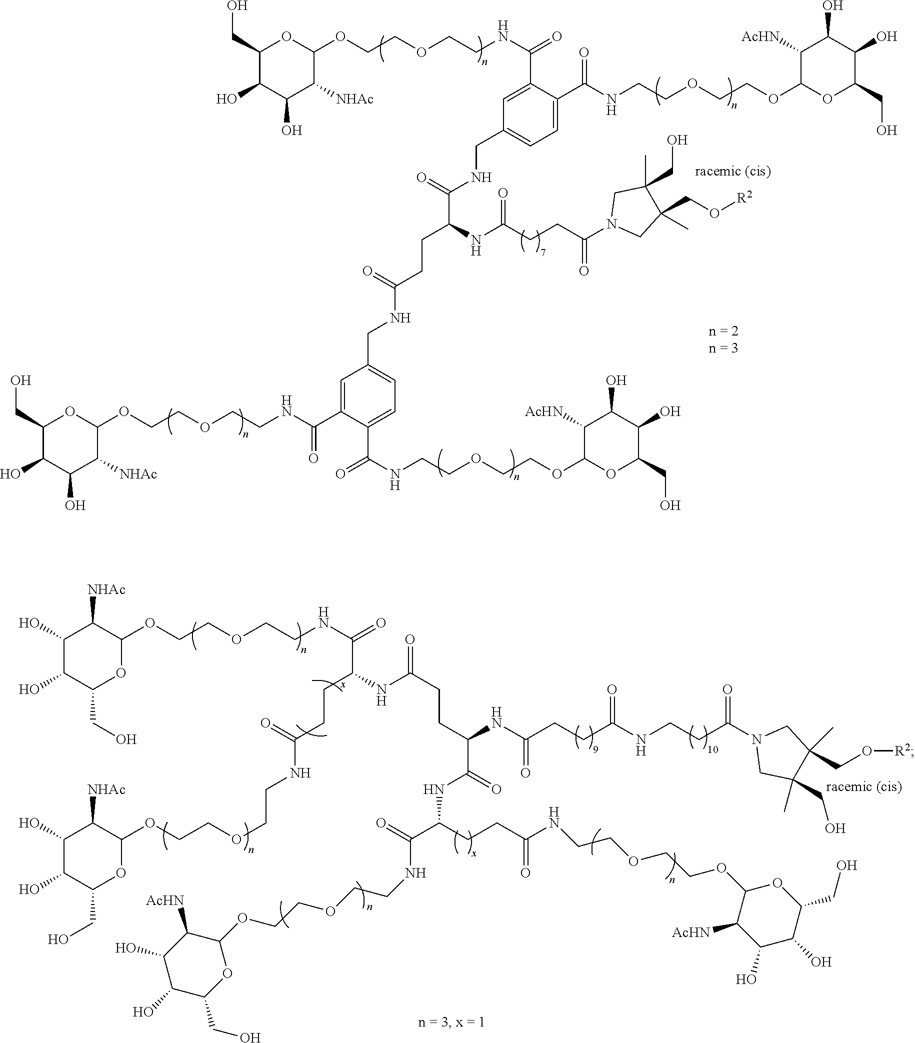

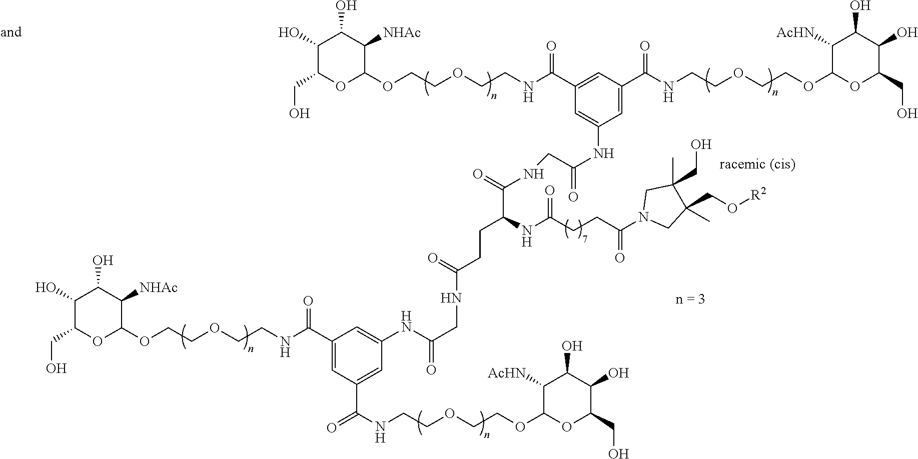

or pharmaceutically acceptable salts thereof, wherein R² is a double stranded siRNA molecule selected from the double stranded siRNA molecules of Table 1 and Table 2.

In one embodiment the compound of formula I is:

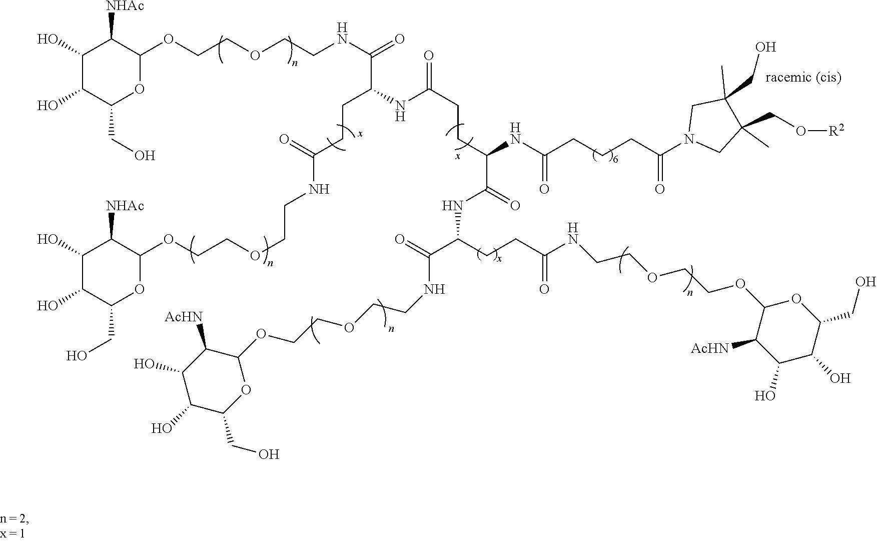

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

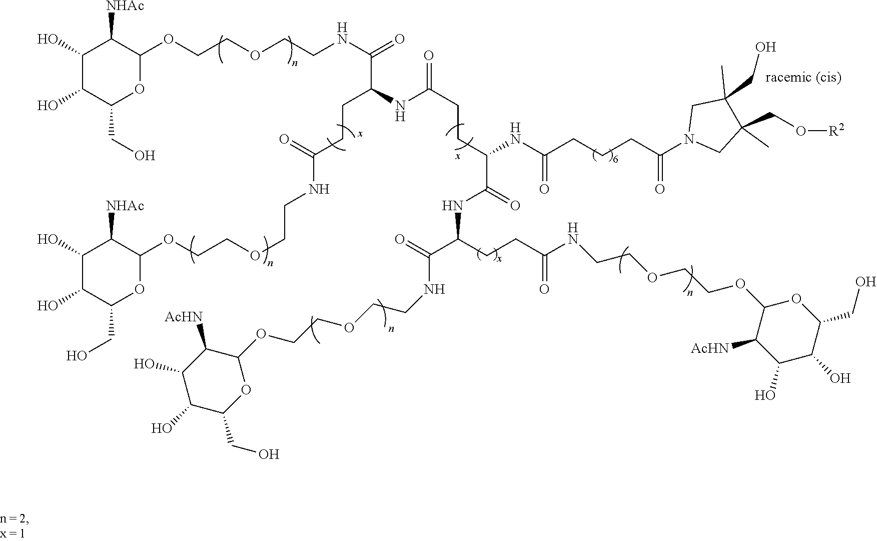

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

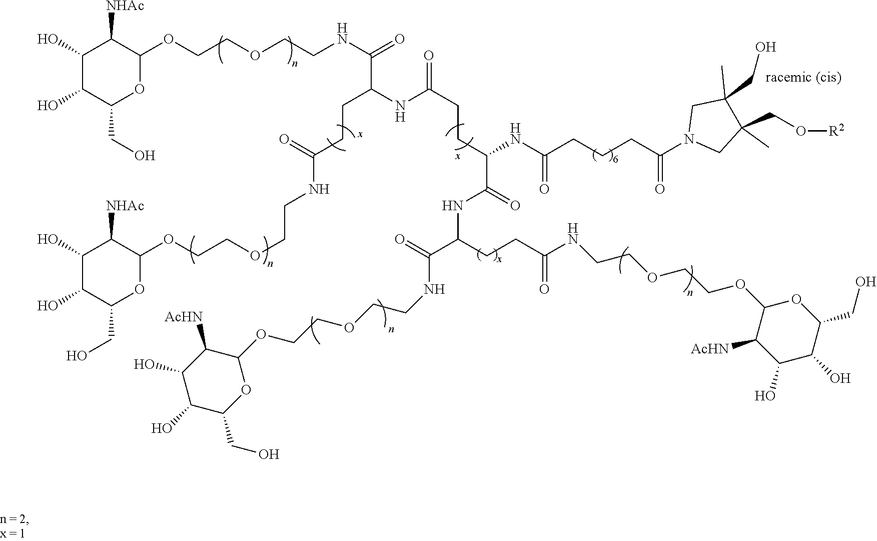

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

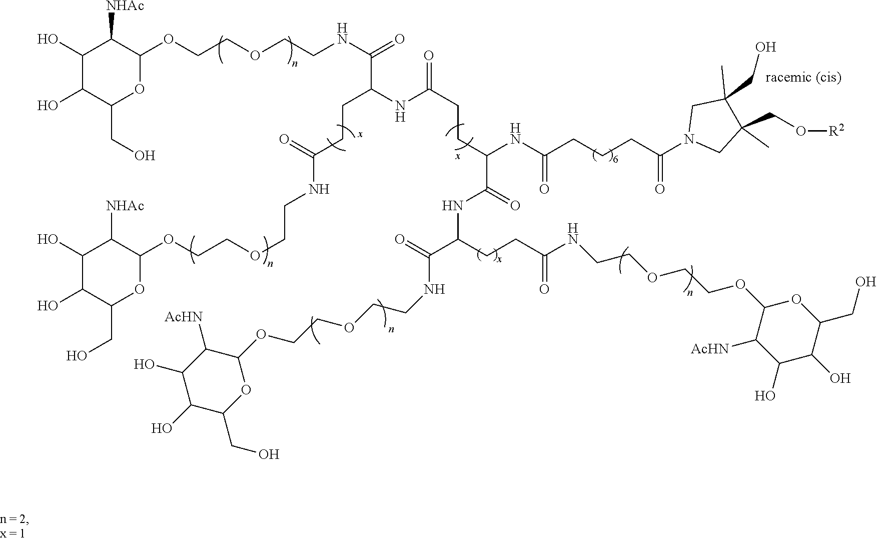

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

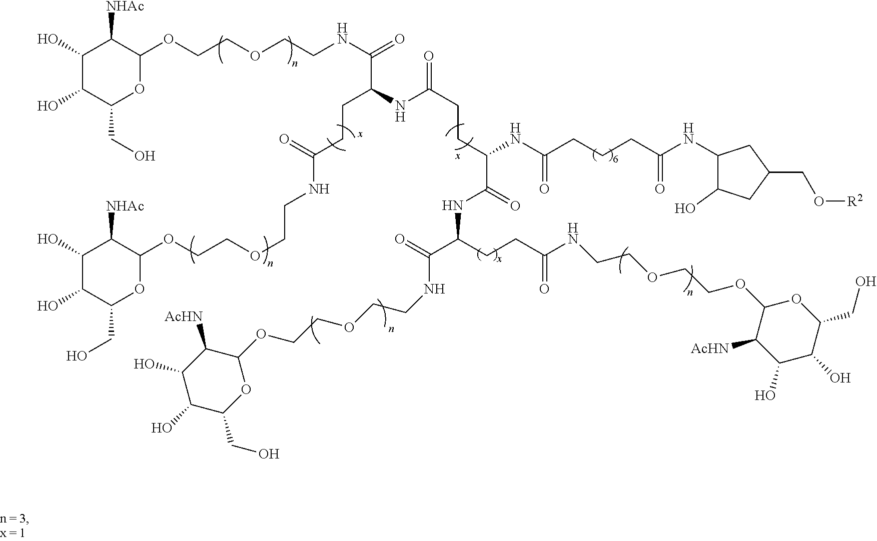

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

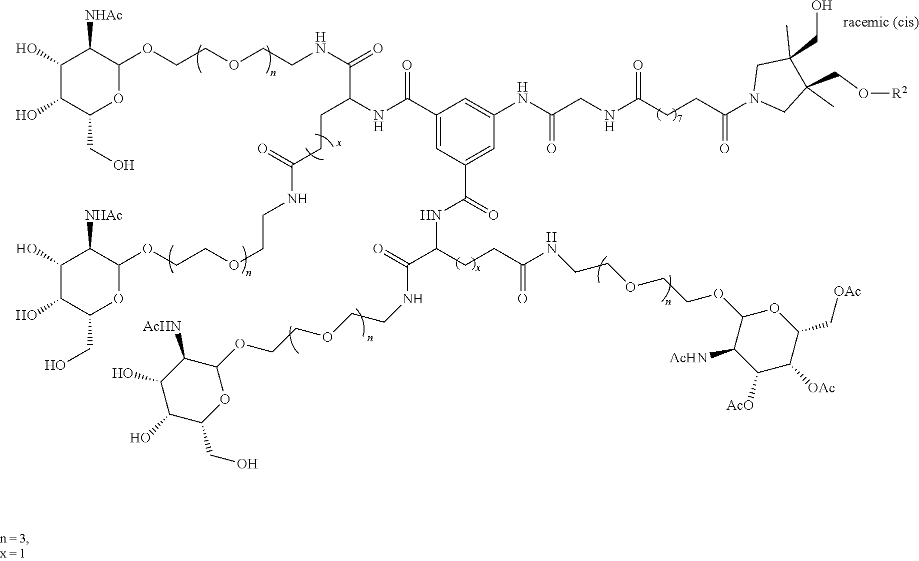

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

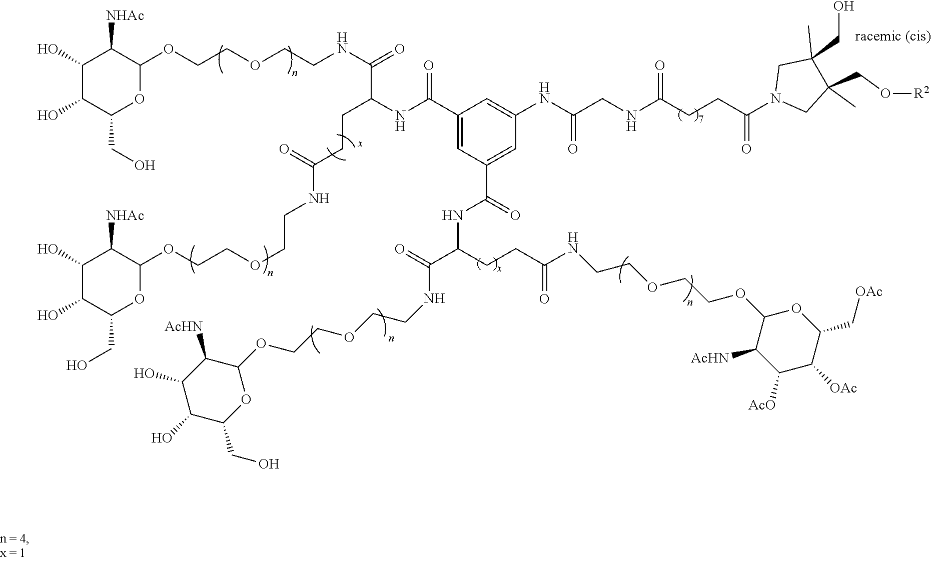

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

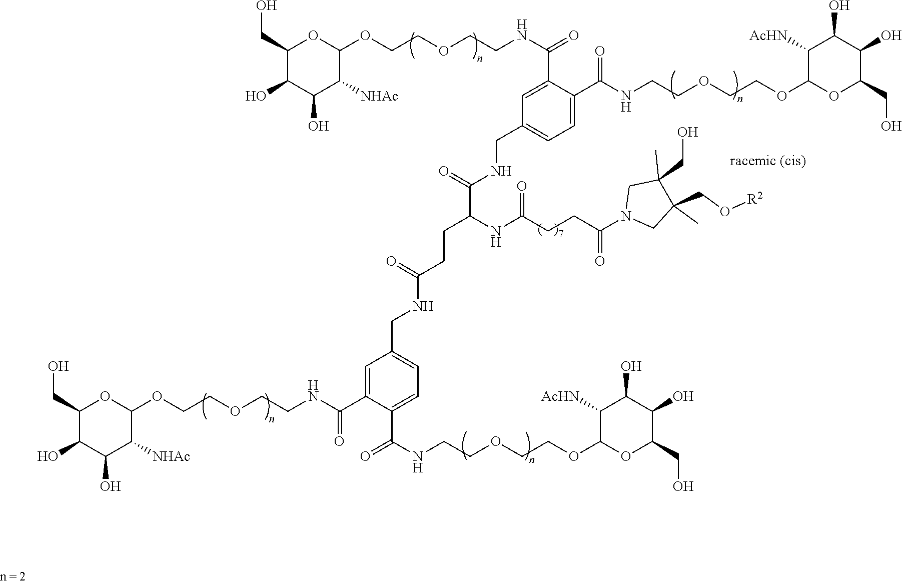

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

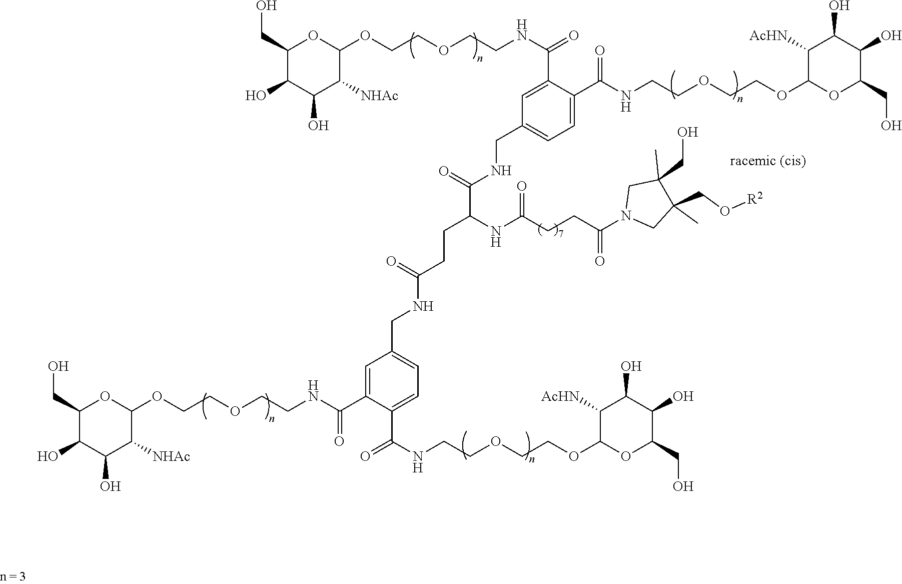

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

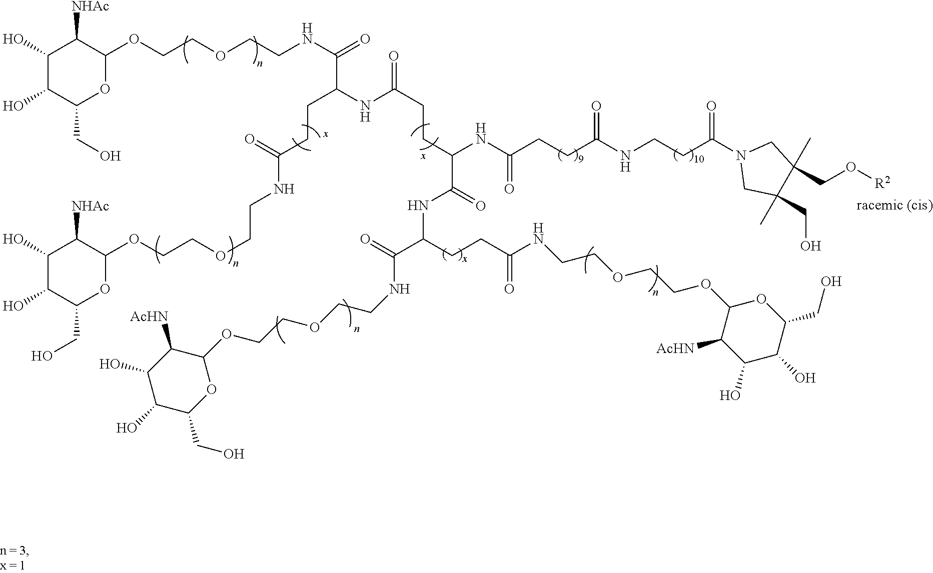

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

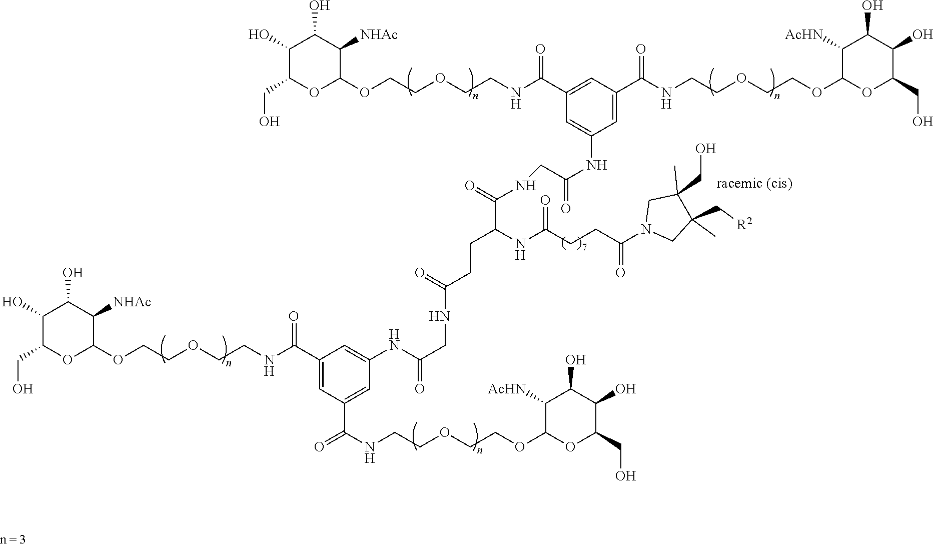

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

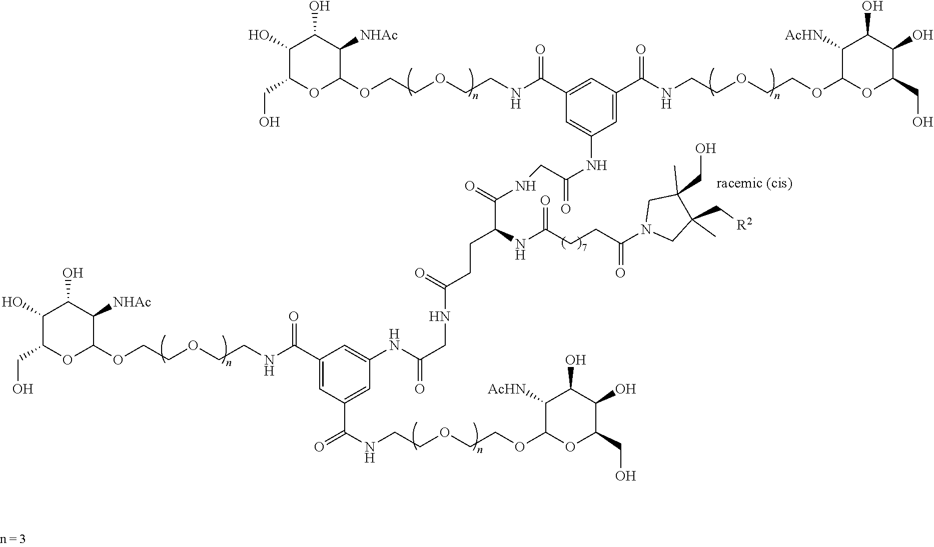

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

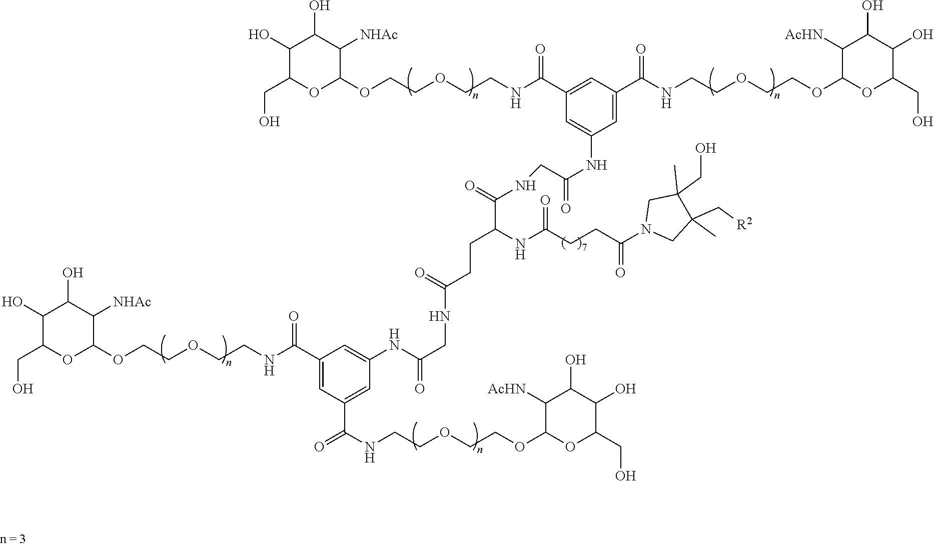

or a pharmaceutically acceptable salt thereof, wherein R² is a double stranded siRNA molecule (e.g. a double stranded siRNA molecule selected from the double stranded siRNA molecules of Table 1 and Table 2).

In one embodiment the compound of formula I is:

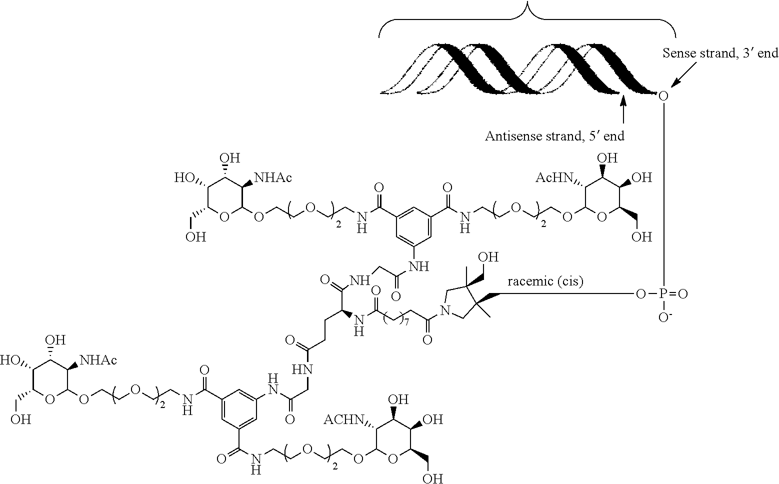

wherein the siRNA is selected from siRNA 1-siRNA 28, or a pharmaceutically acceptable salt thereof.

In one embodiment the compound of formula I is:

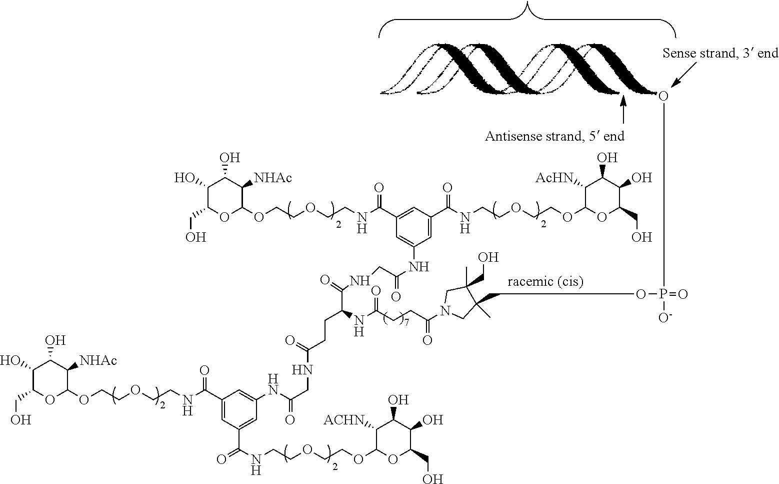

wherein the siRNA is selected from siRNA 1-siRNA 28, or a pharmaceutically acceptable salt thereof.

In one embodiment the compound of formula I is:

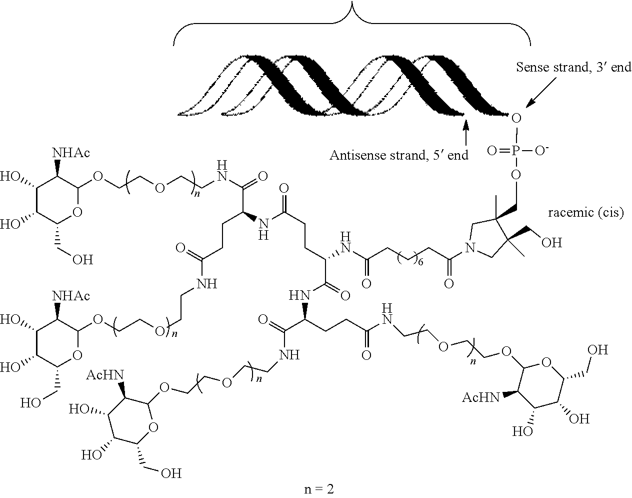

wherein the siRNA is selected from siRNA 1-siRNA 28, or a pharmaceutically acceptable salt thereof.

In one embodiment the compound of formula I is:

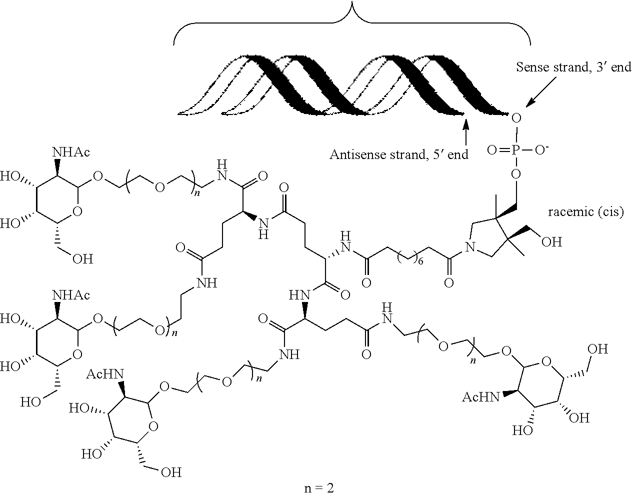

wherein the siRNA is selected from siRNA 1-siRNA 28, or a pharmaceutically acceptable salt thereof.

One embodiment provides a compound of formula (I):

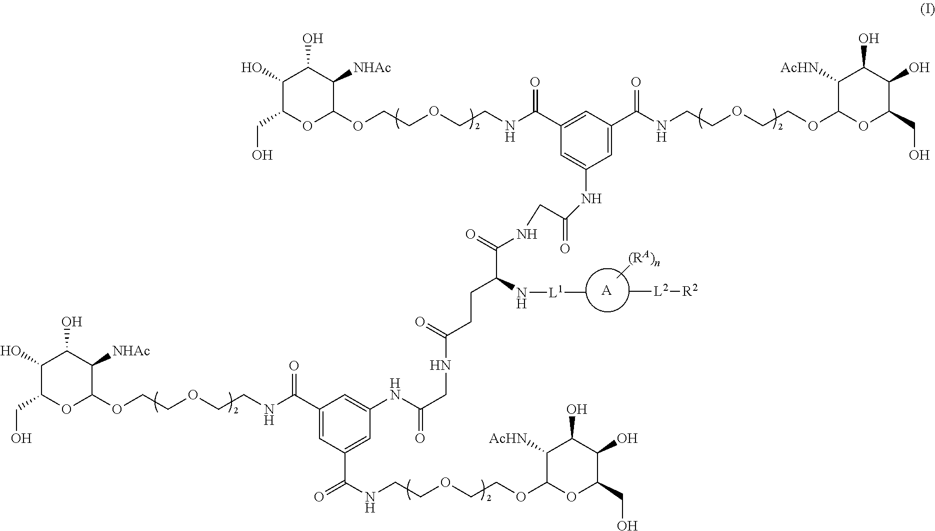

wherein:

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula:

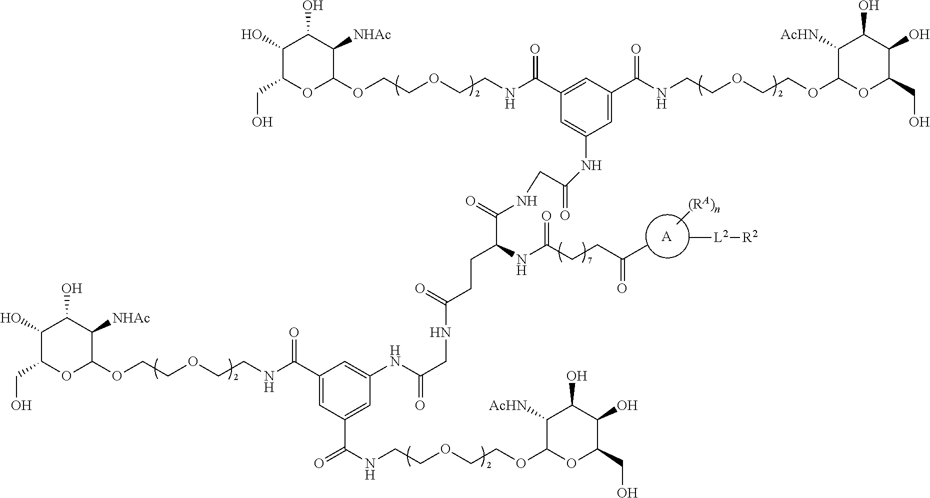

wherein:

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula:

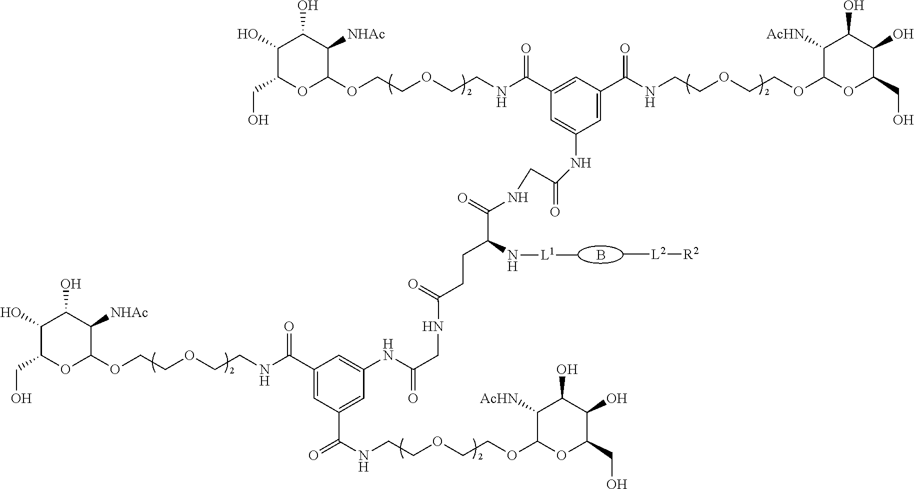

wherein:

L¹ is absent or a linking group;

L² is absent or a linking group;

R² is a nucleic acid;

B is divalent and is selected from the group consisting of:

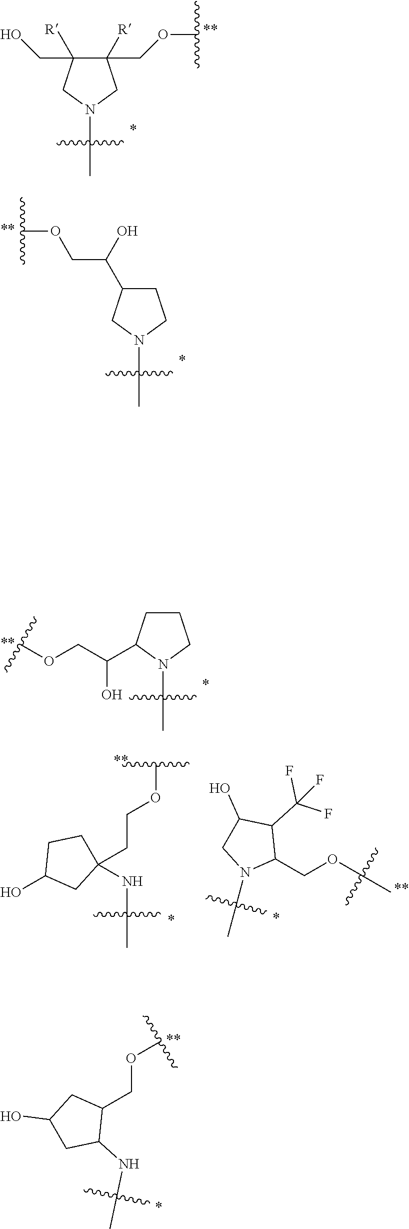

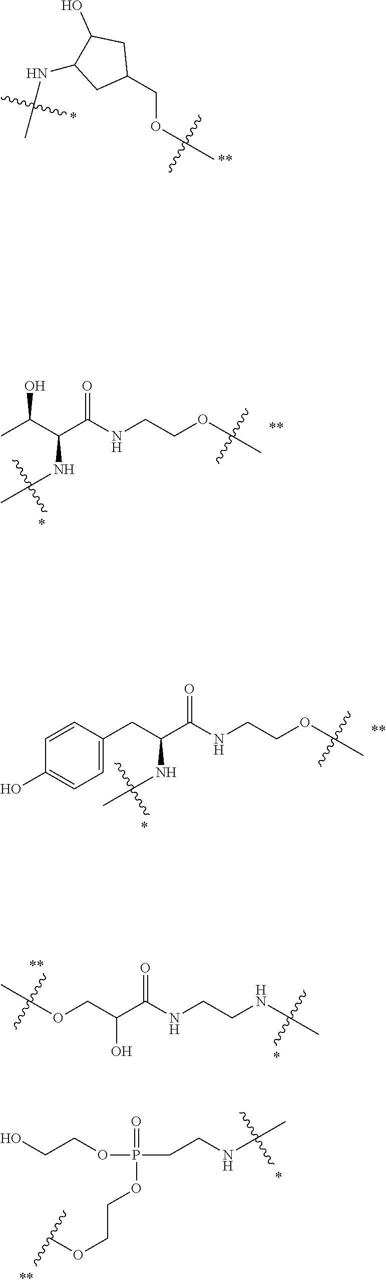

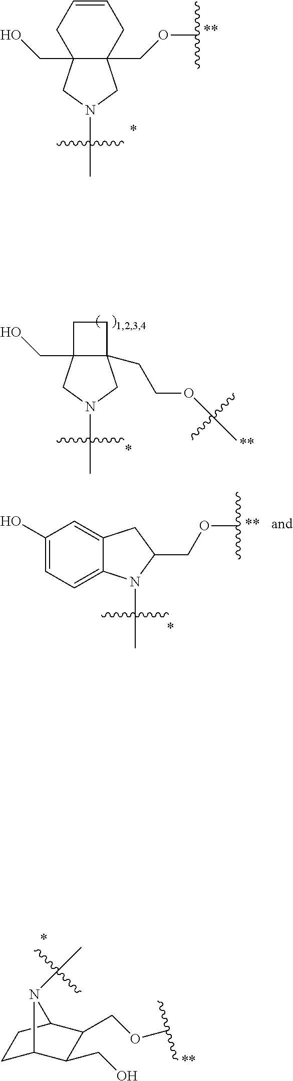

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—NR^{X}— or —S—, and wherein R^{X} is hydrogen or (C1-C6)alkyl, and wherein the hydrocarbon chain, is optionally substituted with one or more substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment L¹ is selected from the group consisting of:

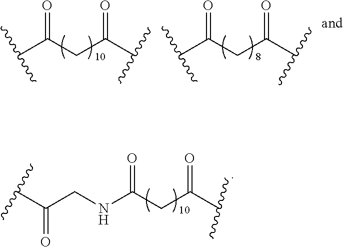

or a salt thereof.

In one embodiment L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

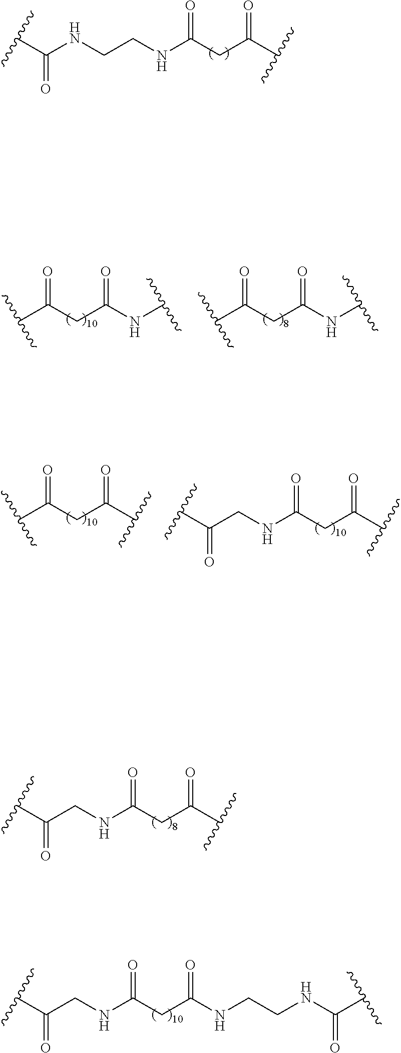

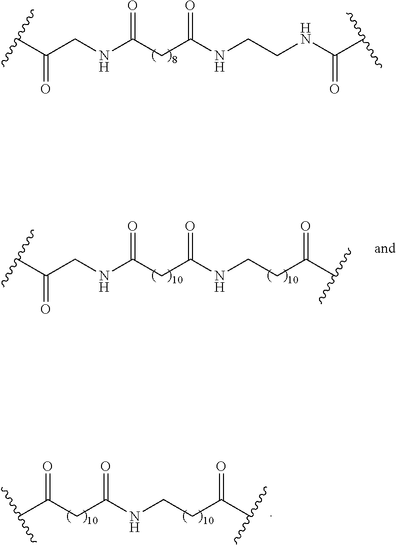

In one embodiment L² is connected to R² through —O—.

In one embodiment L² is C₁₋₄ alkylene-O— that is optionally substituted with hydroxy.

In one embodiment L² is absent.

One embodiment provides a compound,

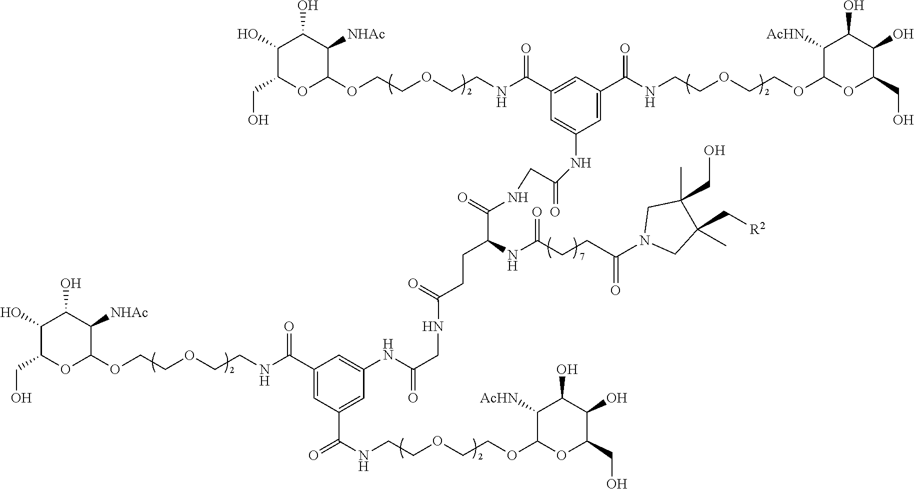

or a salt thereof wherein R² is a nucleic acid.

One aspect is pharmaceutical composition comprising a compound of formula I, and a pharmaceutically acceptable carrier.

Another aspect is a method to deliver a double stranded siRNA to the liver of an animal comprising administering a compound of formula I or a pharmaceutically acceptable salt thereof, to the animal.

Another aspect is a method to treat a disease or disorder (e.g., a liver disease or a viral infection, such as a hepatitis B viral infection) in an animal comprising administering a compound of formula I or a pharmaceutically acceptable salt thereof, to the animal.

Certain embodiments provide a compound of formula (I) or a pharmaceutically acceptable salt thereof for use in medical therapy.

Certain embodiments provide a compound of formula (I) or a pharmaceutically acceptable salt thereof for the prophylactic or therapeutic treatment of a disease or disorder (e.g., a liver disease or a viral infection, such as a hepatitis B virus infection) in an animal.

Certain embodiments provide the use of a compound of formula (I) or a pharmaceutically acceptable salt thereof to prepare a medicament for treating a disease or disorder (e.g., a liver disease or a viral infection, such as a hepatitis B virus infection) in an animal.

In certain embodiments, the animal is a mammal, such as a human.

In one embodiment a compound of formula I has the following formula (Id):

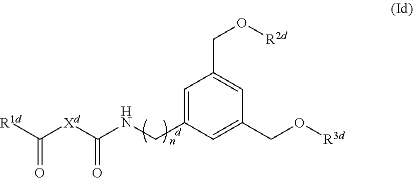

wherein:

R^{1d} is selected from:

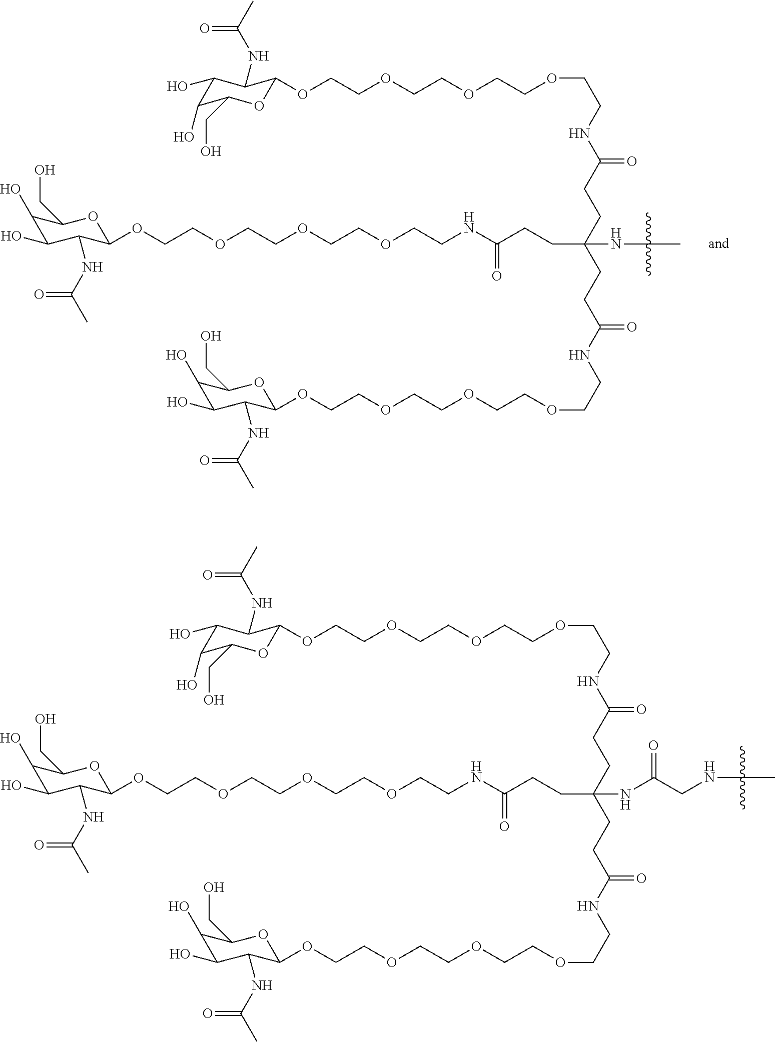 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₁₀ alkylene;
n^{d} is 0 or 1;
R^{2d} is a double stranded siRNA molecule selected from the double stranded siRNA of Table 1 and Table 2; and
R^{3d} is H, a protecting group, a covalent bond to a solid support, or a bond to a linking group that is bound to a solid support.

In one embodiment R^{3d} includes a linking group that joins the remainder of the compound of formula Id to a solid support. The nature of the linking group is not critical provided the compound is a suitable intermediate for preparing a compound of formula Id wherein R^{2d} is a double stranded siRNA molecule selected from the double stranded siRNA of Table 1 and Table 2.

In one embodiment the linker in R^{3d} has a molecular weight of from about 20 daltons to about 1,000 daltons.

In one embodiment the linker in R^{3d} has a molecular weight of from about 20 daltons to about 500 daltons.

In one embodiment the linker in R^{3d} separates the solid support from the remainder of the compound of formula I by about 5 angstroms to about 40 angstroms, inclusive, in length.

In one embodiment the linker in R^{3d} is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment the linker in R^{3d} is —C(═O)CH₂CH₂C(═O)N(H)—.

In one embodiment R^{1d} is:

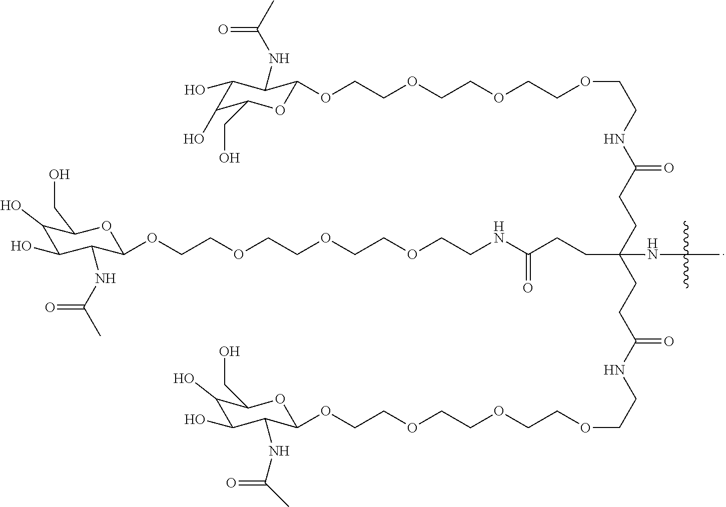

In one embodiment R^{1d} is:

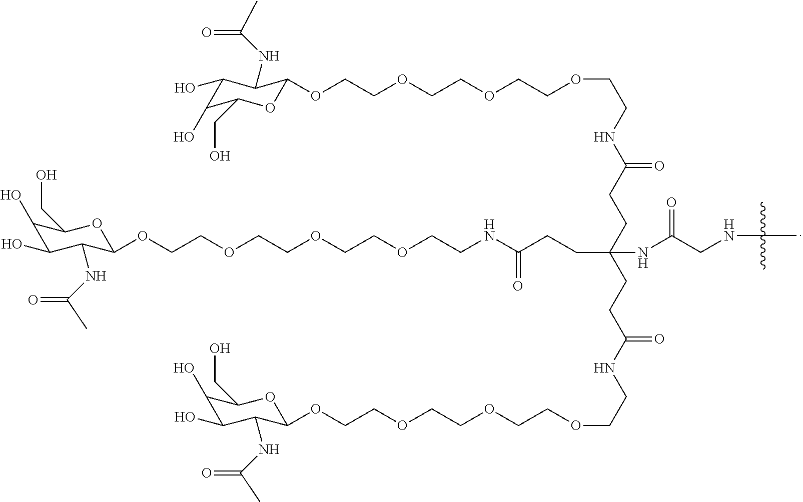 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment X^{d} is C₈alkylene.

In one embodiment n^{d} is 0.

In one embodiment R^{2d} is an siRNA.

In one embodiment R^{3d} is H.

In another embodiment a compound of (Id) or the salt thereof is selected from the group consisting of:

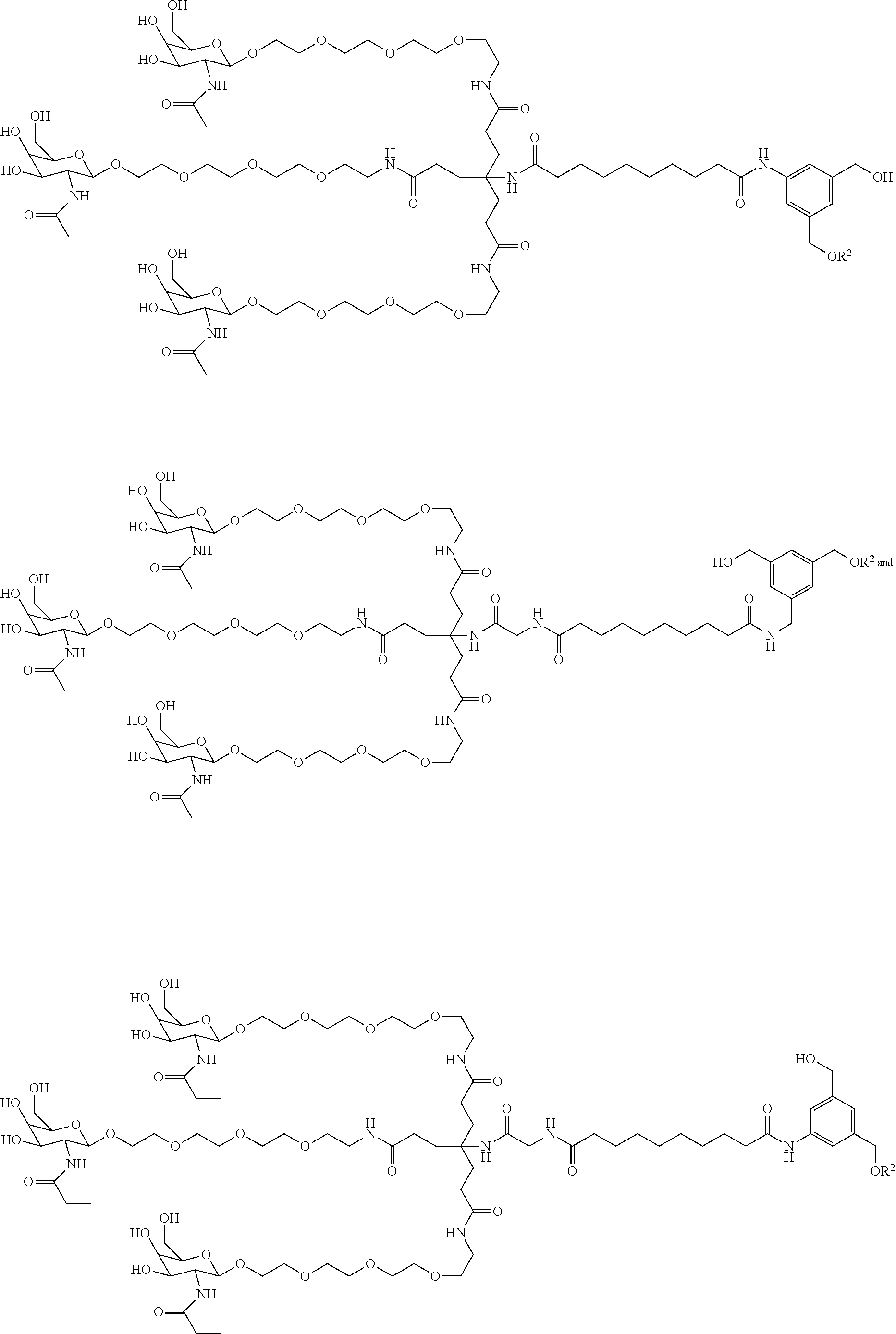

and salts thereof.

One aspect is a pharmaceutical composition comprising a compound of formula (Id), and a pharmaceutically acceptable carrier.

One aspect is a method to deliver is a double stranded siRNA to the liver of an animal comprising administering a compound of formula (Id) or a pharmaceutically acceptable salt thereof, to the animal.

Another aspect is a method to treat a disease or disorder (e.g., a viral infection, such as a hepatitis B viral infection) in an animal comprising administering a compound of formula (Id) or a pharmaceutically acceptable salt thereof, to the animal.

Certain embodiments provide a compound of formula (Id) or a pharmaceutically acceptable salt thereof for use in medical therapy.

Certain embodiments provide a compound of formula (Id) or a pharmaceutically acceptable salt thereof for the prophylactic or therapeutic treatment of a disease or disorder (e.g., a viral infection, such as a hepatitis B virus infection) in an animal.

Certain embodiments provide the use of a compound of formula (Id) or a pharmaceutically acceptable salt thereof to prepare a medicament for treating a disease or disorder (e.g., a viral infection, such as a hepatitis B virus infection) in an animal.

In certain embodiments, the animal is a mammal, such as a human.

Also provided is a method to prepare a compound of formula (Id) as described herein comprising subjecting a corresponding compound of formula (Ie):

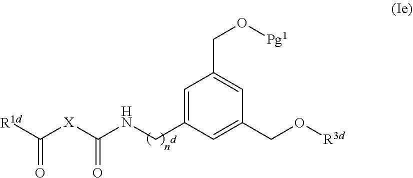

wherein:

X^{d} is C₂₋₈ alkylene;

n^{d} is 0 or 1;

Pg¹ is H; and

R^{3d} is a covalent bond to a solid support or a bond to a linking group that is bound to a solid support, to solid phase nucleic acid synthesis conditions to provide a corresponding compound of formula Id wherein R^{2d} is a double stranded siRNA molecule selected from the double stranded siRNA molecules of Table 1 and Table 2.

In one embodiment the method further comprises removing the compound from the solid support to provide the corresponding compound of formula Id wherein R^{3d} is H.

In one embodiment the compound is not a compound formula Ie:

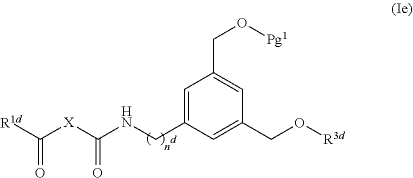

or a salt thereof, wherein:

R^{1d} is selected from:

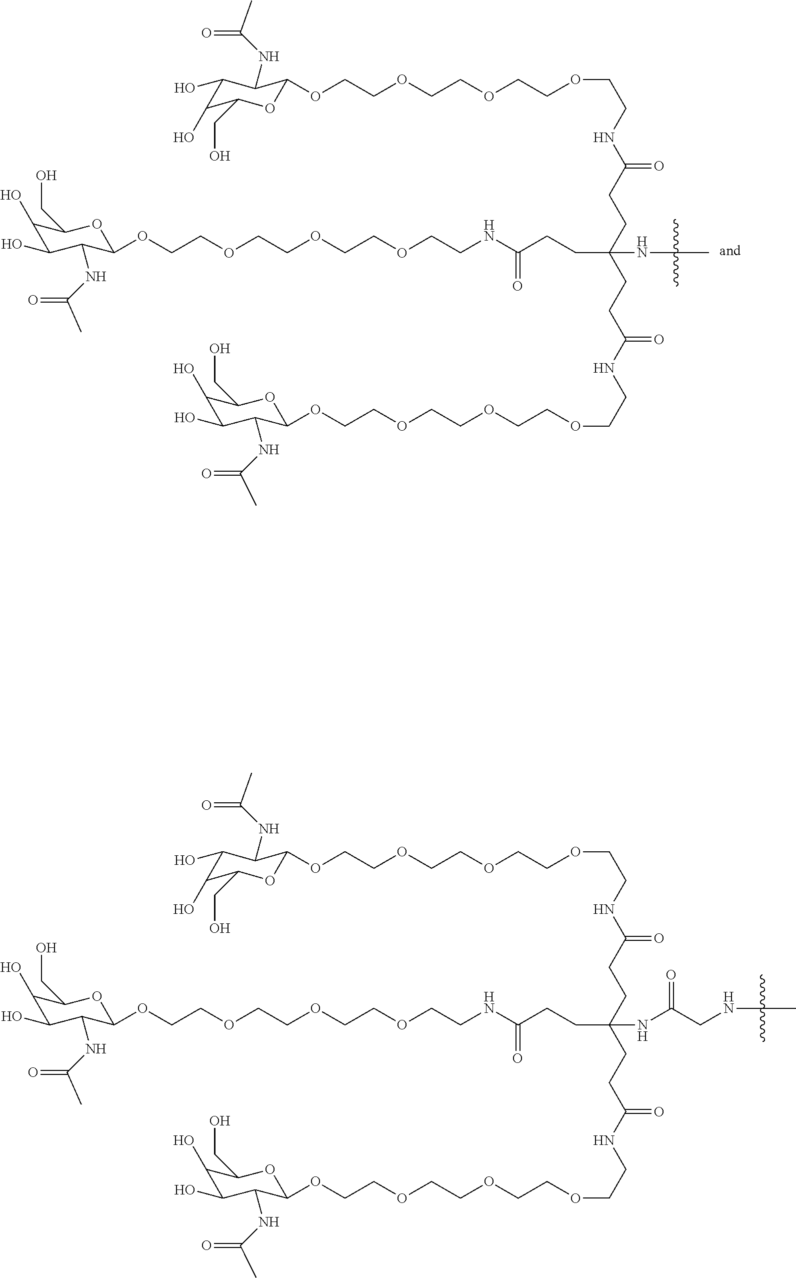 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

X^{d} is C₂₋₈ alkylene;

n^{d} is 0 or 1;

Pg¹ is H or a suitable protecting group; and

R^{3d} is H, a protecting group, a covalent bond to a solid support, or a bond to a linking group that is bound to a solid support.

In one embodiment R^{3d} is H.

In one embodiment R^{3d} is a covalent bond to a solid support.

In one embodiment R^{3d} is a bond to a linking group that is bound to a solid support, wherein the linking group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—) or (—N(H)—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment R^{3d} is a bond to a linking group that is bound to a solid support, wherein the linking group is —C(═O)CH₂CH₂C(═O)N(H)—.

One embodiment provides a compound of formula (I):

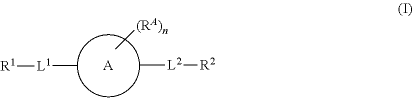

wherein:

R¹ is H or a synthetic activating group;

L¹ is absent or a linking group;

L² is absent or a linking group;

R² is a double stranded siRNA molecule selected from the double stranded siRNA molecules of Table 1 and Table 2;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

One embodiment provides a compound of formula (Ig):

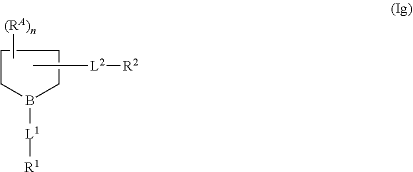

wherein:

B is or —CH—;

L² is C₁₋₄ alkylene-O— that is optionally substituted with hydroxyl or halo; and

n is 0, 1, 2, 3, 4, 5, 6, or 7;

or a salt thereof.

One embodiment provides a compound selected from the group consisting of:

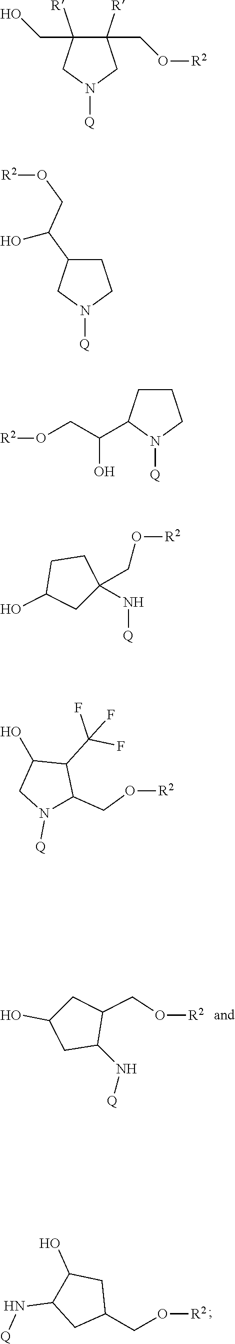

wherein:

Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

and salts thereof.

One embodiment provides a compound selected from the group consisting of:

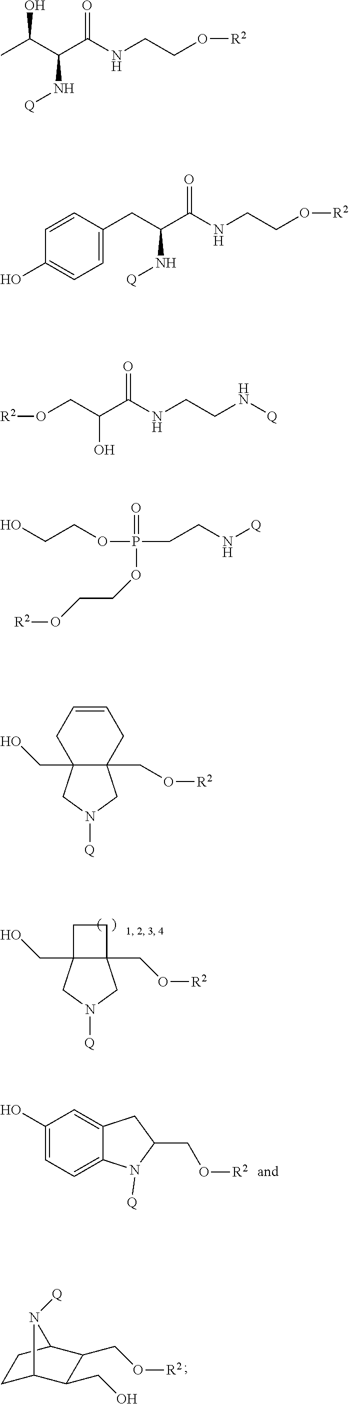

wherein: Q is -L¹-R¹; and salts thereof.

In one embodiment L¹ is a divalent, branched or unbranched, saturated or unsaturated, hydrocarbon chain, having from 5 to 20 carbon atoms, wherein one or more (e.g. 1, 2, 3, or 4) of the carbon atoms in the hydrocarbon chain is optionally replaced —O—, —NH—, —NH—C(═O)—, —C(═O)—NH— or —S—.

One embodiment provides a compound of formula (XX):

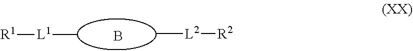

wherein:

R¹ a is targeting ligand;

L¹ is absent or a linking group;

L² is absent or a linking group;

R² is a double stranded siRNA molecule selected from the double stranded siRNA molecules of Table 1 and Table 2;

B is divalent and is selected from the group consisting of:

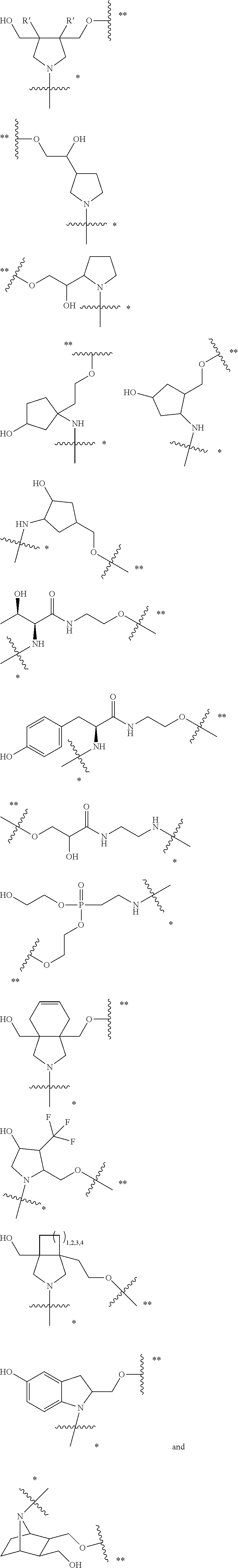

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent;

or a salt thereof.

In one embodiment R¹ comprises 2-8 saccharides.

In one embodiment R¹ comprises 2-6 saccharides.

In one embodiment R¹ comprises 2-4 saccharides.

In one embodiment R¹ comprises 3-8 saccharides.

In one embodiment R¹ comprises 3-6 saccharides.

In one embodiment R¹ comprises 3-4 saccharides.

In one embodiment R¹ comprises 3 saccharides.

In one embodiment R¹ comprises 4 saccharides.

In one embodiment R¹ has the following formula:

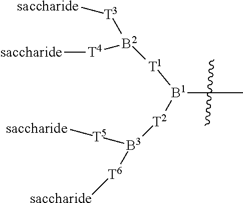

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶;

T¹ is absent or a linking group;

T² is absent or a linking group;

T³ is absent or a linking group;

T⁴ is absent or a linking group;

T⁵ is absent or a linking group; and

T⁶ is absent or a linking group

In one embodiment each saccharide is independently selected from:

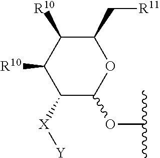

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In one embodiment each saccharide is independently selected from the group consisting of:

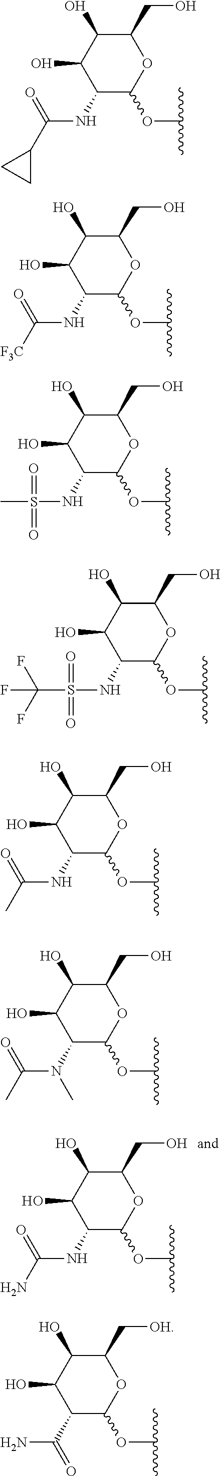

In one embodiment each saccharide is independently:

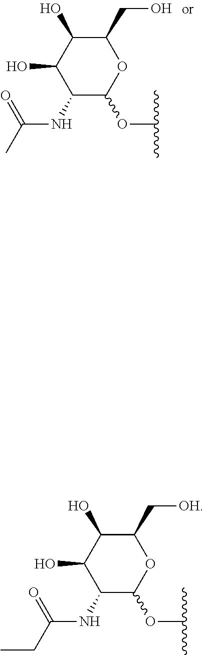 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment one of T¹ and T² is absent.

In one embodiment both T¹ and T² are absent.

In one embodiment each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more (e.g. 1, 2, 3, or 4) of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment at least one of T³, T⁴, T⁵, and T⁶ is:

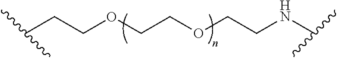

wherein:

n=1, 2, 3.

In one embodiment each of T³, T⁴, T⁵, and T⁶ is independently selected from the group consisting of:

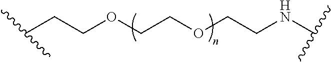

wherein:

n=1, 2, 3.

In one embodiment at least one of T¹ and T² is glycine

In one embodiment each of T¹ and T² is glycine.

In one embodiment B¹ is a trivalent group comprising 1 to 15 atoms and is covalently bonded to L¹, T¹, and T².

In one embodiment B¹ is a trivalent group comprising 1 to 10 atoms and is covalently bonded to L¹, T¹, and T².

In one embodiment B¹ comprises a (C₁-C₆)alkyl.

In one embodiment B¹ comprises a C₃₋₈ cycloalkyl.

In one embodiment B¹ comprises a silyl group.

In one embodiment B¹ comprises a D- or L-amino acid.

In one embodiment B¹ comprises a saccharide.

In one embodiment B¹ comprises a phosphate group.

In one embodiment B¹ comprises a phosphonate group.

In one embodiment B¹ comprises an aryl.

In one embodiment B¹ comprises a phenyl ring.

In one embodiment B¹ is a phenyl ring.

In one embodiment B¹ is CH.

In one embodiment B¹ comprises a heteroaryl.

In one embodiment B¹ is selected from the group consisting of:

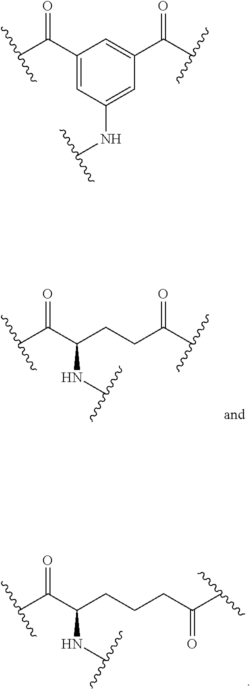 [SMILES: *Nc1cc(C(*)=O)cc(C(*)=O)c1 ; *N[C@H](CCC(*)=O)C(*)=O ; *N[C@H](CCCC(*)=O)C(*)=O]

In one embodiment B¹ is selected from the group consisting of:

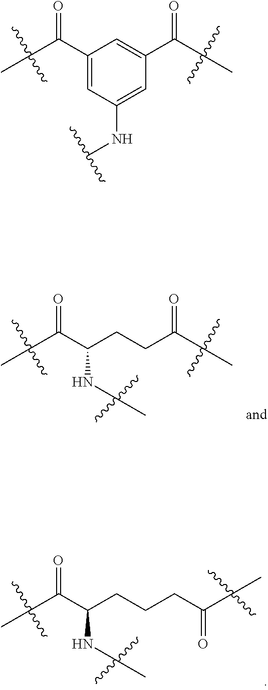 [SMILES: *Nc1cc(C(*)=O)cc(C(*)=O)c1 ; *N[C@@H](CCC(*)=O)C(*)=O ; *N[C@H](CCCC(*)=O)C(*)=O]

In one embodiment B² is a trivalent group comprising 1 to 15 atoms and is covalently bonded to L¹, T¹, and T².

In one embodiment B² is a trivalent group comprising 1 to 10 atoms and is covalently bonded to L¹, T¹, and T².

In one embodiment B² comprises a (C₁-C₆)alkyl

In one embodiment B² comprises a C₃₋₈ cycloalkyl.

In one embodiment B² comprises a silyl group.

In one embodiment B² comprises a D- or L-amino acid.

In one embodiment B² comprises a saccharide.

In one embodiment B² comprises a phosphate group.

In one embodiment B² comprises a phosphonate group.

In one embodiment B² comprises an aryl.

In one embodiment B² comprises a phenyl ring.

In one embodiment B² is a phenyl ring.

In one embodiment B² is CH.

In one embodiment B² comprises a heteroaryl.

In one embodiment B² is selected from the group consisting of:

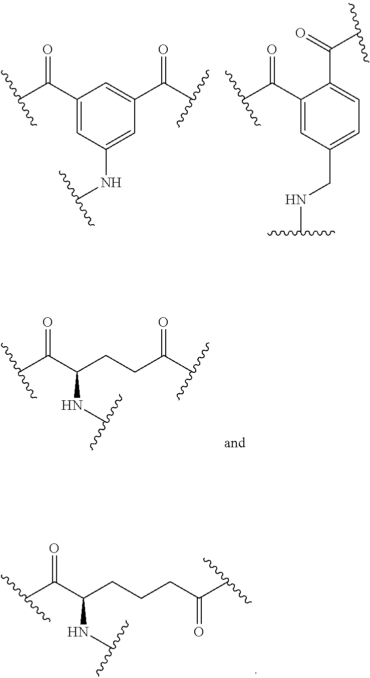 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B² is selected from the group consisting of:

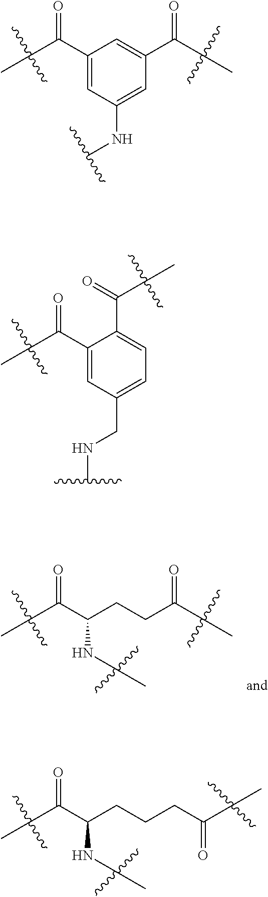 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment B³ is a trivalent group comprising 1 to 15 atoms and is covalently bonded to L¹, T¹, and T².

In one embodiment B³ is a trivalent group comprising 1 to 10 atoms and is covalently bonded to L¹, T¹, and T².

In one embodiment B³ comprises a (C₁-C₆)alkyl.

In one embodiment B³ comprises a C₃₋₈ cycloalkyl.

In one embodiment B³ comprises a silyl group.

In one embodiment B³ comprises a D- or L-amino acid.

In one embodiment B³ comprises a saccharide.

In one embodiment B³ comprises a phosphate group.

In one embodiment B³ comprises a phosphonate group.

In one embodiment B³ comprises an aryl.

In one embodiment B³ comprises a phenyl ring.

In one embodiment B³ is a phenyl ring.

In one embodiment B³ is CH.

In one embodiment B³ comprises a heteroaryl.

In one embodiment B³ is selected from the group consisting of:

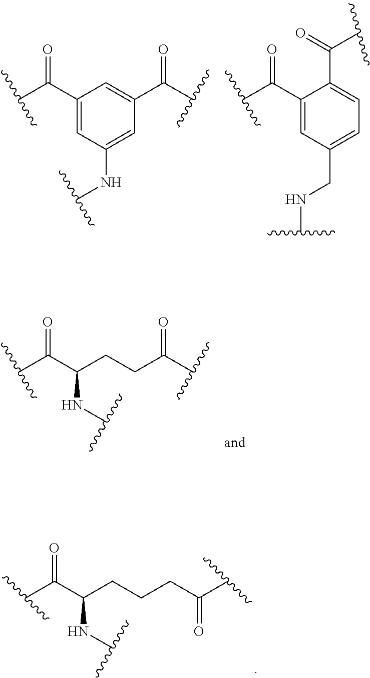 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment B³ is selected from the group consisting of:

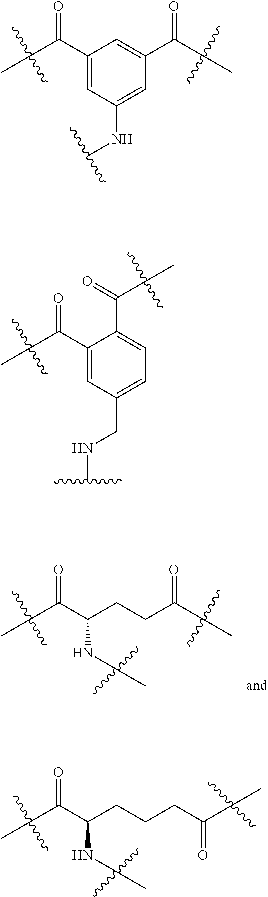 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment L¹ is selected from the group consisting of:

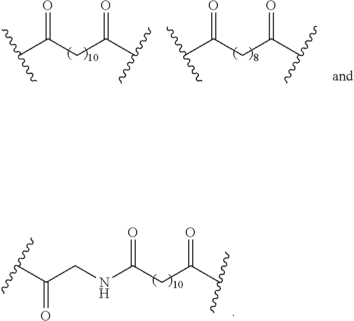

or a salt thereof.

In one embodiment L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment L¹ is selected from the group consisting of:

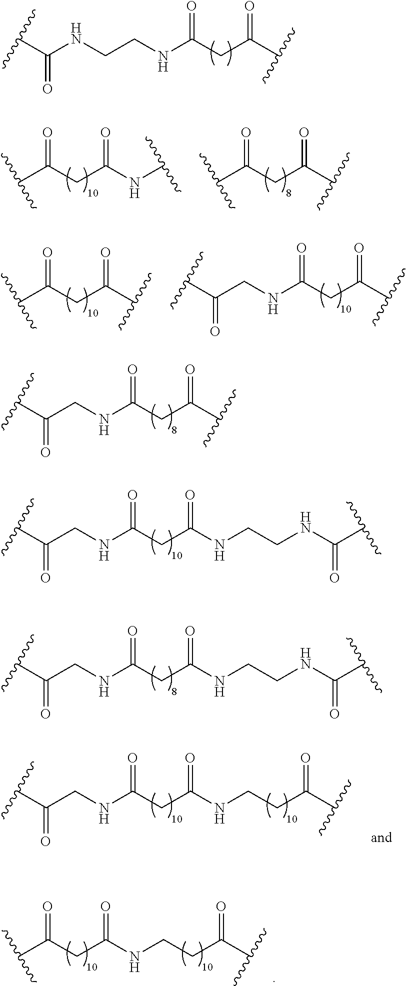

In one embodiment L² is connected to R² through —O—.

In one embodiment L² is C₁₋₄ alkylene-O— that is optionally substituted with hydroxy.

In one embodiment L² is connected to R² through —O—.

In one embodiment L² is absent.

One embodiment provides a compound or salt selected from the group consisting of:

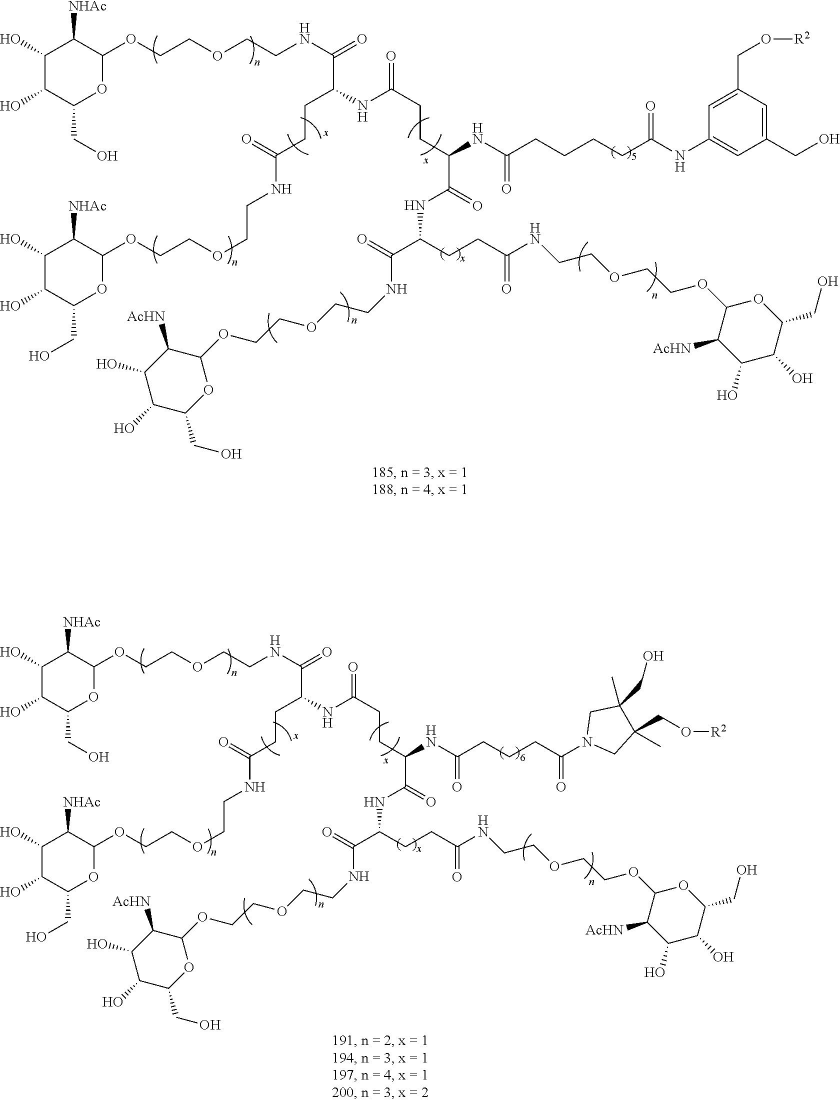

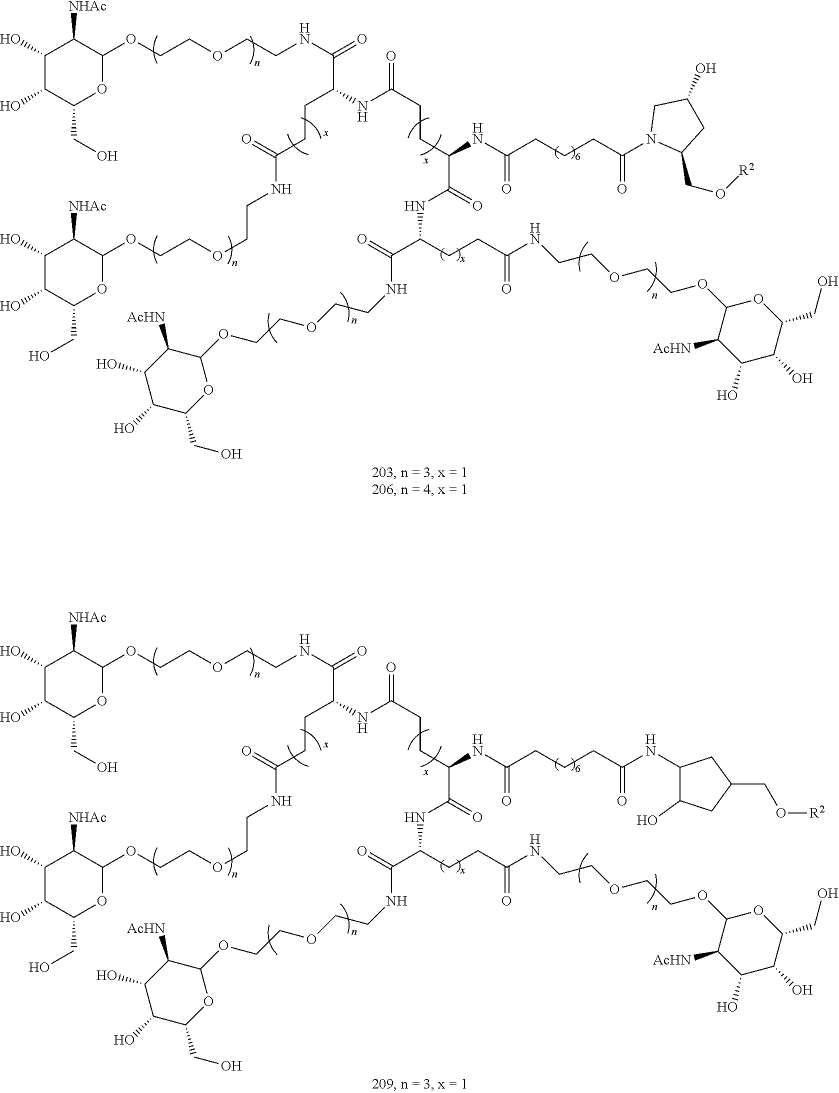

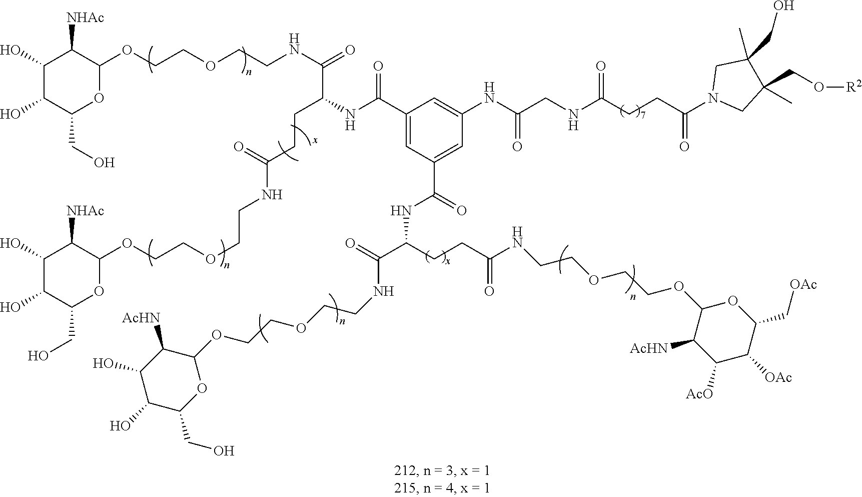

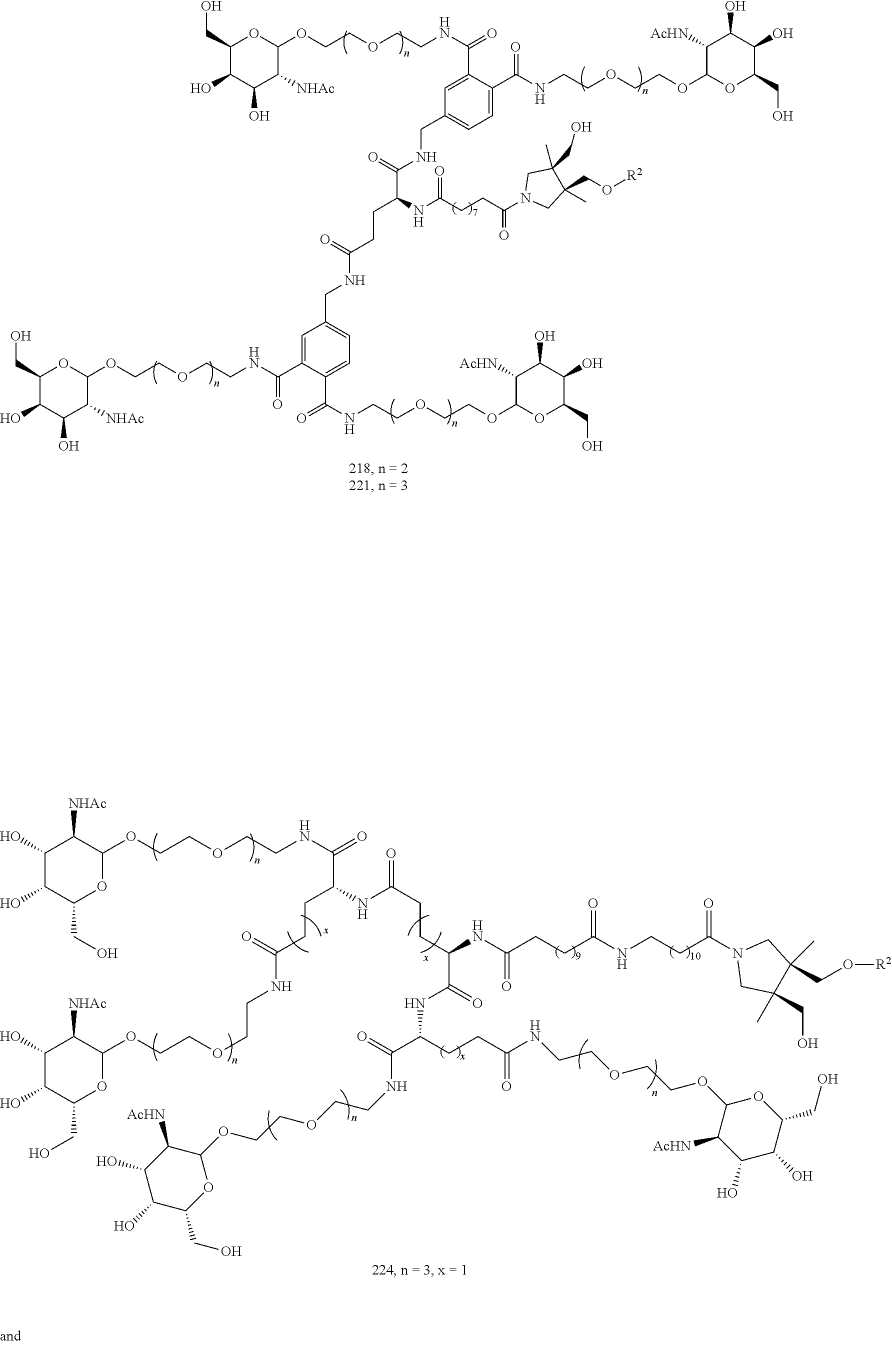

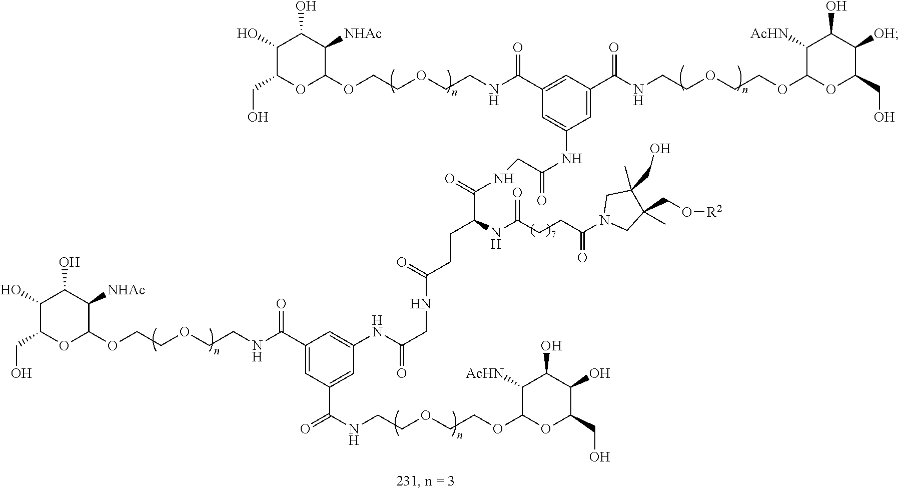

and pharmaceutically acceptable salts thereof, wherein R² is a double stranded siRNA molecule selected from the double stranded siRNA molecules of Table 1 and Table 2.

One embodiment provides a compound of formula:

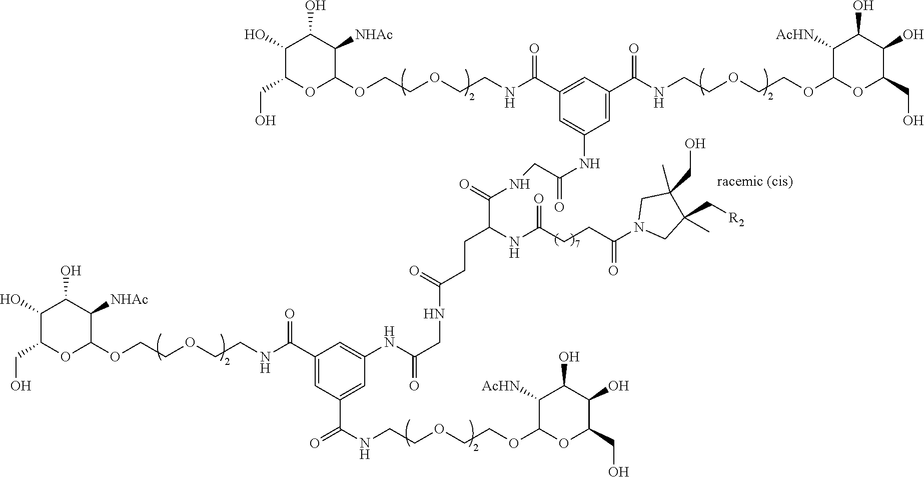

or a salt thereof wherein R² is a nucleic acid.

One embodiment provides a compound of formula:

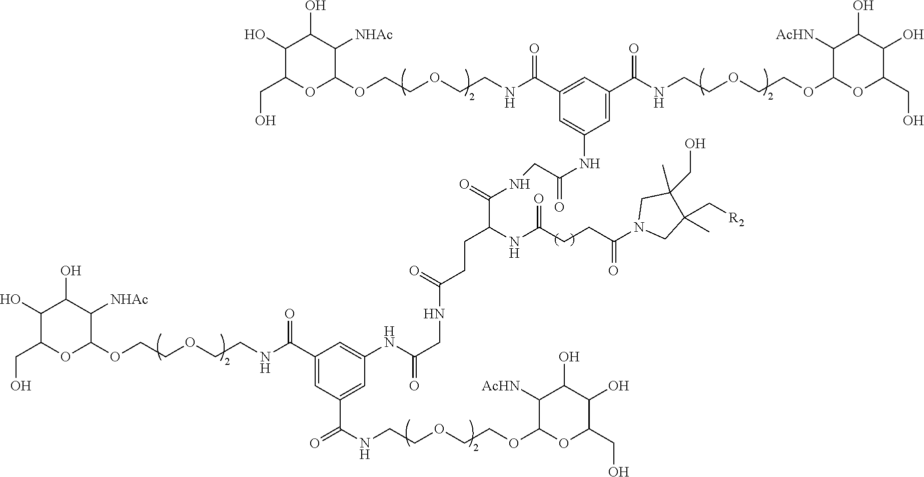

or a salt thereof wherein R² is a nucleic acid.

In one embodiment, the nucleic acid molecule (e.g., siRNA) is attached to the reminder of the compound through the oxygen of a phosphate at the 3′-end of the sense strand.

In one embodiment the compound or salt is administered subcutaneously.

When a compound comprises a group of the following formula:

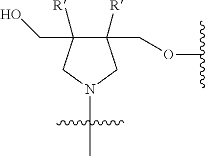

there are four stereoisomers possible on the ring, two cis and two trans. Unless otherwise noted, the compounds include all four stereoisomers about such a ring. In one embodiment, the two R′ groups are in a cis conformation. In one embodiment, the two R′ groups are in a trans conformation.

One aspect is a nucleic acid-lipid particle comprising:
(a) one or more double stranded siRNA molecules selected from the double stranded siRNA molecules of Table 1 and Table 2;
(b) a cationic lipid; and
(c) a non-cationic lipid.

### EXAMPLES

The present invention will be described in greater detail by way of specific examples.

The following examples are offered for illustrative purposes and are not intended to limit the invention in any manner. Those of skill in the art will readily recognize a variety of noncritical parameters which can be changed or modified to yield essentially the same results. It is understood that in one embodiment the oligonucleotide is a double stranded siRNA molecule as described in Table 1 or Table 2.

### Cell Culture and Direct Incubations:

Primary human hepatocytes obtained from Primacyt (Schwerin, Germany) were thawed, plated and cultured according to manufacturer's instructions. Cells were grown at 37° C. in an atmosphere with 5% CO2 in a humidified incubator.

For direct incubation with GalNAc-siRNA conjugates, primary human hepatocytes were generally seeded at a density of 90,000 cells/well in 96-well plates in total volume of 90 μl plating medium. Standard dose-response screening experiments, e.g., for GalNAc-hsGHR siRNAs, were done with final siRNA concentrations of 5, 2.5, 1.25, 0.625, 0.3125, 0.15625, 0.078125, 0.0390625, 0.01953125 and 0.0097656525 μM. Control wells were either treated with medium only or directly incubated with a GalNAc-hsAPOC3 targeting siRNA. Oligonucleotide stocks were diluted in plating medium, a volume of 10 μl of diluted oligonucleotide was added to 90 μl cell suspension. Five hours post-treatment, the cell culture supernatant was carefully removed followed by addition of 50 μl complete growth medium. The media was again exchanged 24 h post-treatment followed by incubation of cells for yet another 24 h at 37° C./5% CO2 in humidified incubator.

### Branched DNA Assays QuantiGene 2.0

After a total period of 48-hour incubation, media was removed and cells were lysed in 1500 Lysis Mixture (1 volume lysis mixture provided by QuantiGene, 2 volumes nuclease-free water), then incubated at 53° C. for 60 minutes. 900 Working Probe Set hsGHR (gene target), 80 μl Working Probe Set hsAPOC3 (positive control for uptake via Asialoglycoprotein Receptor) and 90 μl Working Probe Set GAPDH (endogenous control) and 20 μl or 10 μl of cell-lysate were then added to the Capture Plates resulting in a total volume per well of 100 μl. Sealed Capture Plates were incubated at 53° C. for all samples (approx. 16-20 hrs). The next day, the Capture Plates were washed 3 times with at least 300 μl of 1× Wash Buffer (nuclease-free water, Buffer Component 1 and Wash Buffer Component 2). After the last wash, the plate was inverted and blotted against clean paper towels. 100 μl of pre-Amplifier Working Reagent was added to the hsGHR and hsAPOC3 Capture Plates, which were sealed with aluminum foil and incubated for 1 hour at 53° C. Following a 1-hour incubation, the wash step was repeated, then 100 μl Amplifier Working Reagent was added to hsGHR, hsAPOC3 and hsGAPDH capture plates. After 1 hour of incubation at 53° C., the wash and dry steps were repeated, and 100 μl Label Probe was added. Capture plates were incubated at 53° C. for 1 hour. The plates were then washed with 1× Wash Buffer and dried, and then 100 μl Substrate was added to the Capture Plates. Luminescence was read using 1420 Luminescence Counter (WALLAC VICTOR Light, Perkin Elmer, Rodgau-Jügesheim, Germany) following 30 minutes incubation in the dark.

bDNA Data Analysis

For each hsGHR siRNA or hsAPOC3 control siRNA or medium only treatments, four wells were incubated in parallel, and individual data points were collected from each well. For each well, the hsGHR (or hsAPOC3) mRNA level was normalized to the hsGAPDH mRNA level. The activity of a given hsGHR (or hsAPOC3) siRNA was expressed as percent hsGHR (or hsAPOC3) mRNA concentration (normalized to hsGAPDH mRNA) in treated cells, relative to the hsGHR (or hsAPOC3) mRNA concentration (normalized to hsGAPDH mRNA) averaged across control wells.

### Conjugates

As described herein, various conjugates can be used in the practice of the invention. For Examples 1-3 herein, the following conjugate was used. Additional conjugates useful with the siRNA molecules described herein are described in WO 2017/177326 (PCT/CA2017/050447) and in WO 2018/191278 (PCT/US2018/026918), the disclosures of which are each incorporated by reference.

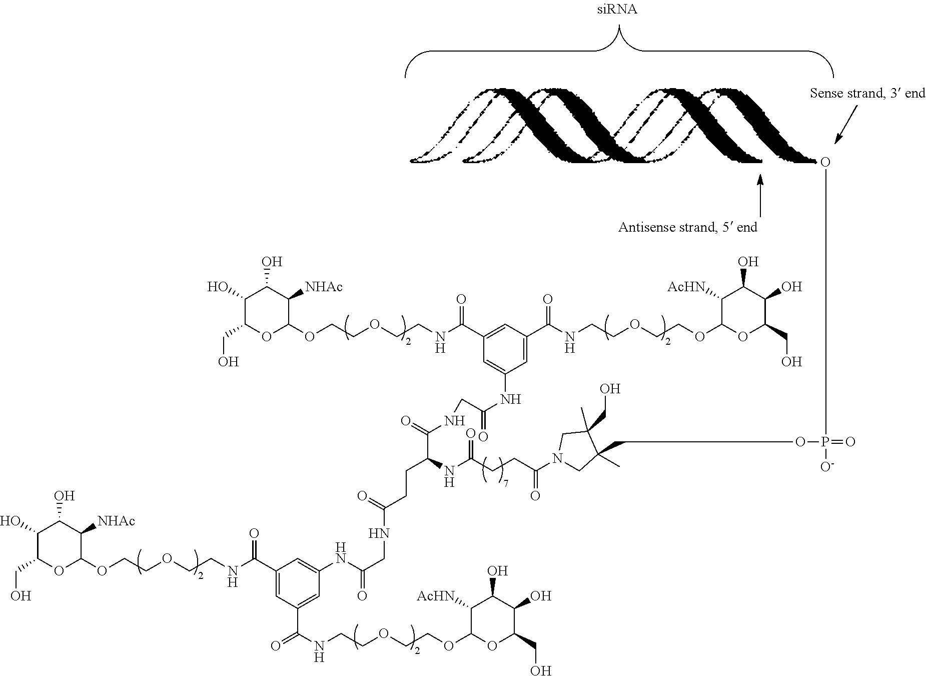

siRNA Sequences

siRNA sequences used in the present Examples are depicted in Tables 1 and 2.

### Example 1

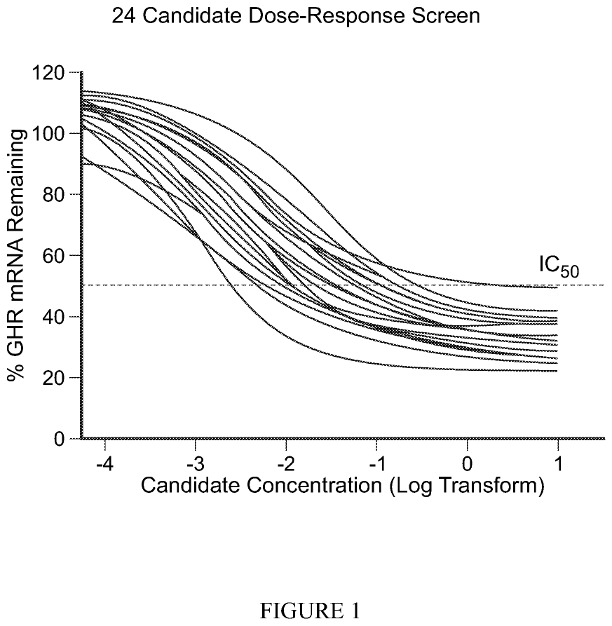

As depicted in FIG. 1, the dose-response of 24 GalNAc-conjugated siRNA in PHHs was evaluated (siRNA 1-siRNA 24). Increasing concentrations of each siRNA were incubated with primary human hepatocytes for 48 hours, with delivery being GalNAc-dependent. GHR mRNA was assayed by qPCR.

### Example 2

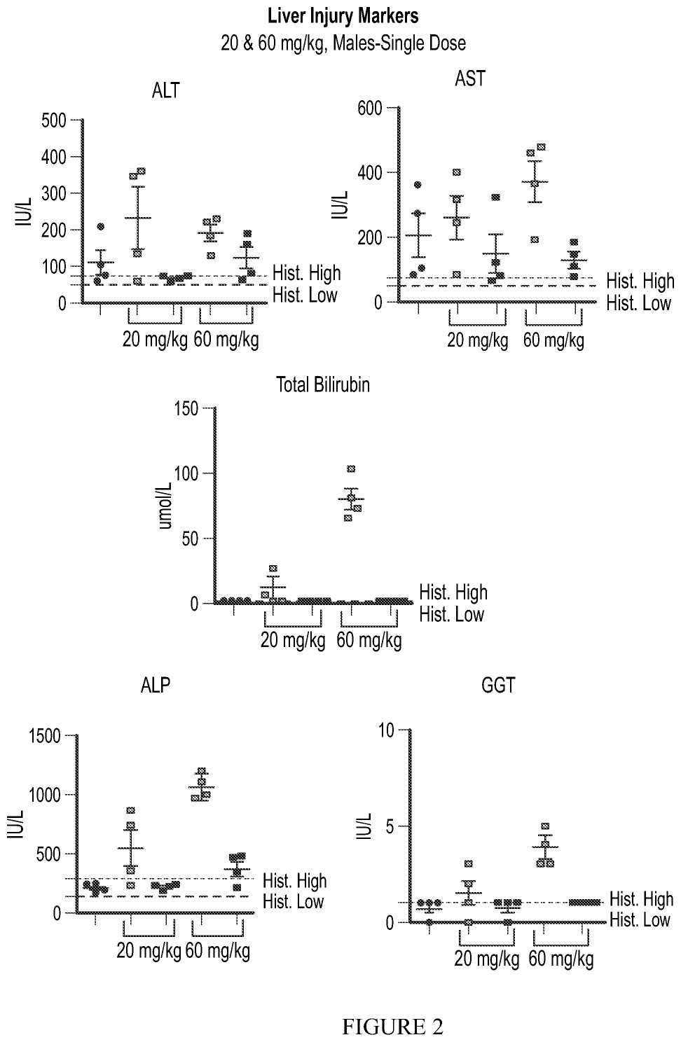

As depicted in FIG. 2, liver injury markers after a single-dose of GHR-targeting candidates were measured. Male rats received a single sub-cutaneous injection of the indicated siRNA at 20 or 60 mg/kg. Serum markers of liver injury were analyzed 14-days post-dose.

### Example 3

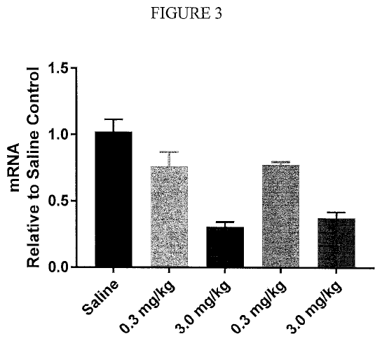

As depicted in FIG. 3, GHR mRNA reduction in NHPs after a single administration of siRNA was measured. Cynomolgus macaques were administered the indicated dosage of each clinical candidate subcutaneously. 14-days post-dose, liver biopsies were taken and GHR mRNA levels were assayed by qPCR.

What is claimed is:

1. A compound of formula (I): 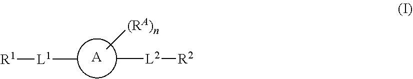
wherein:
R¹ a is targeting ligand;
L¹ is absent or a linking group;
L² is absent or a linking group;
R² is a siRNA molecule selected from any one of siRNA 1-siRNA 28;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen or a protecting group; and
n is 0, 1, 2, 3, 4, 5, 6, 7, 8, 9, or 10;
or a salt thereof.

2. The compound of claim 1, wherein R¹ is —C(H)₍₃₋ₚ₎(L³-saccharide)ₚ;
wherein each L³ is independently a linking group;
p is 1, 2, or 3; and
saccharide is a monosaccharide or disaccharide
or a salt thereof.

3. The compound of claim 2, wherein the saccharide is: 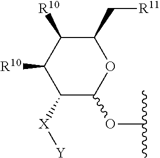
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy;
or a salt thereof.

4. The compound of claim 2 or 3, wherein the saccharide is selected from the group consisting of: 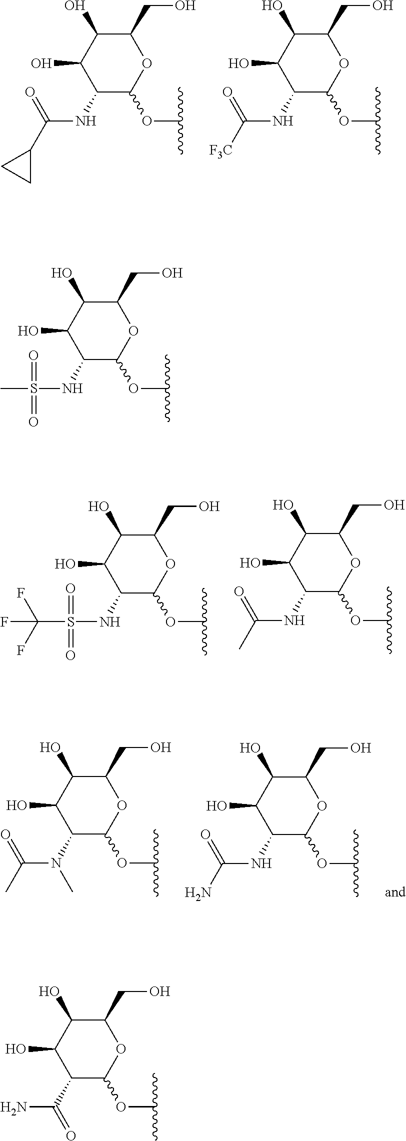
or a salt thereof.

5. The compound of any one of claims 2-4, wherein the saccharide is: 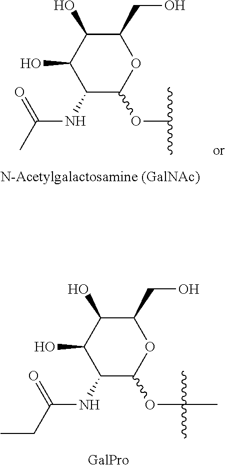 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]
or a salt thereof.

6. The compound of claim 1, wherein the compound of formula I is selected from the group consisting of: 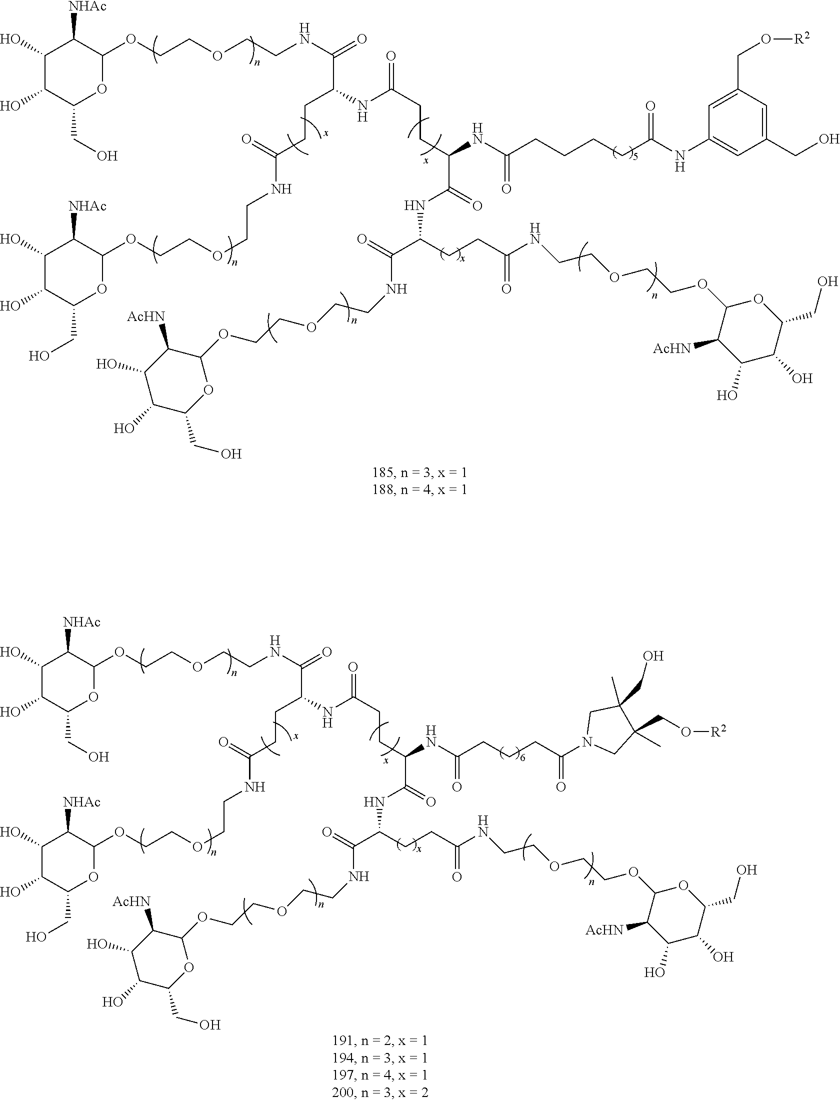 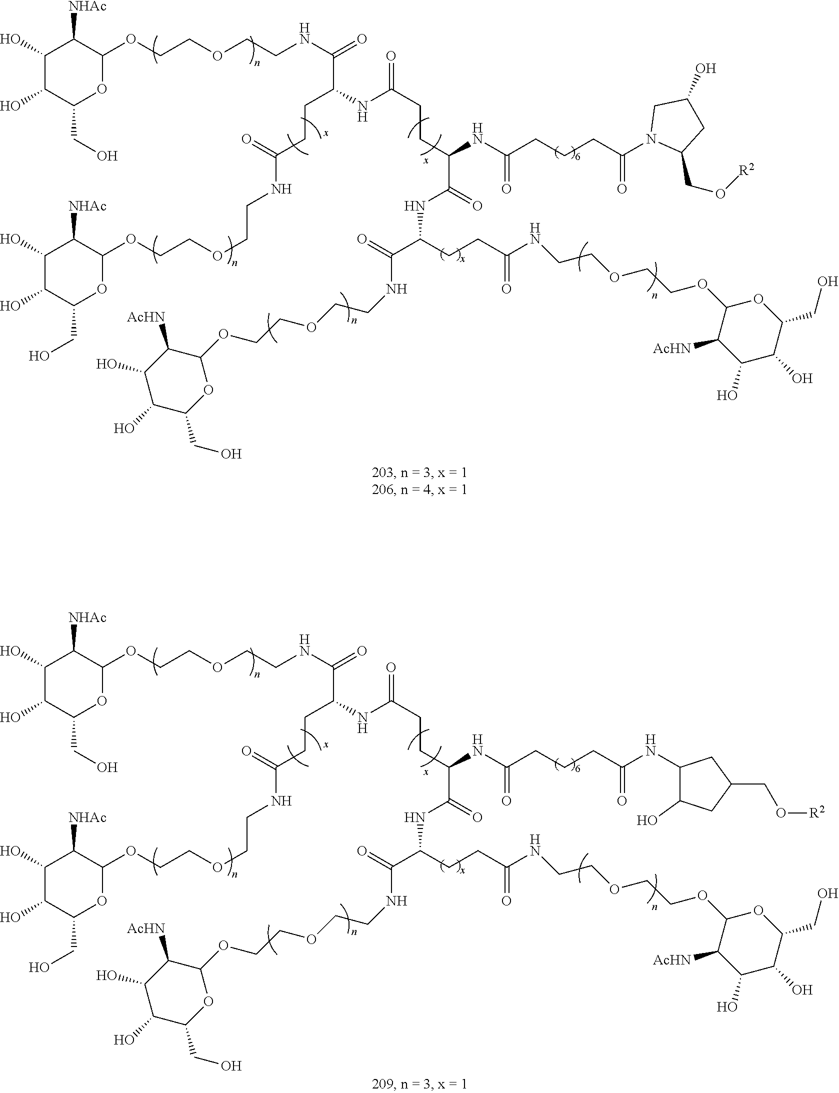 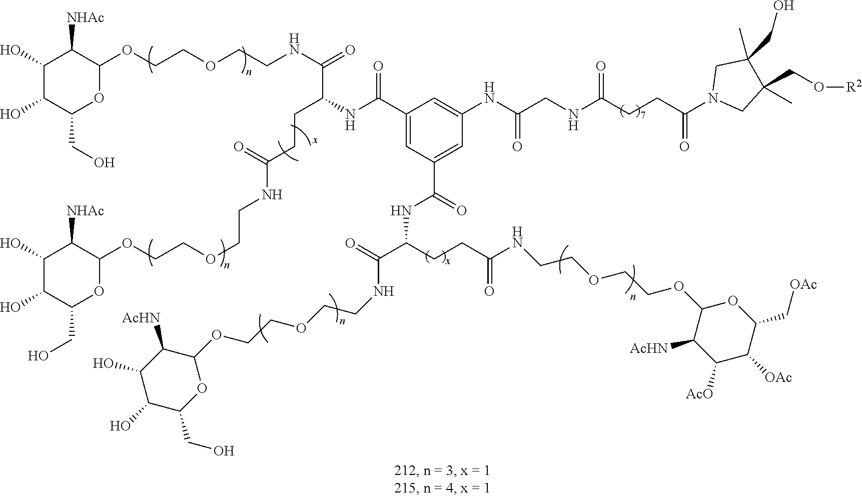 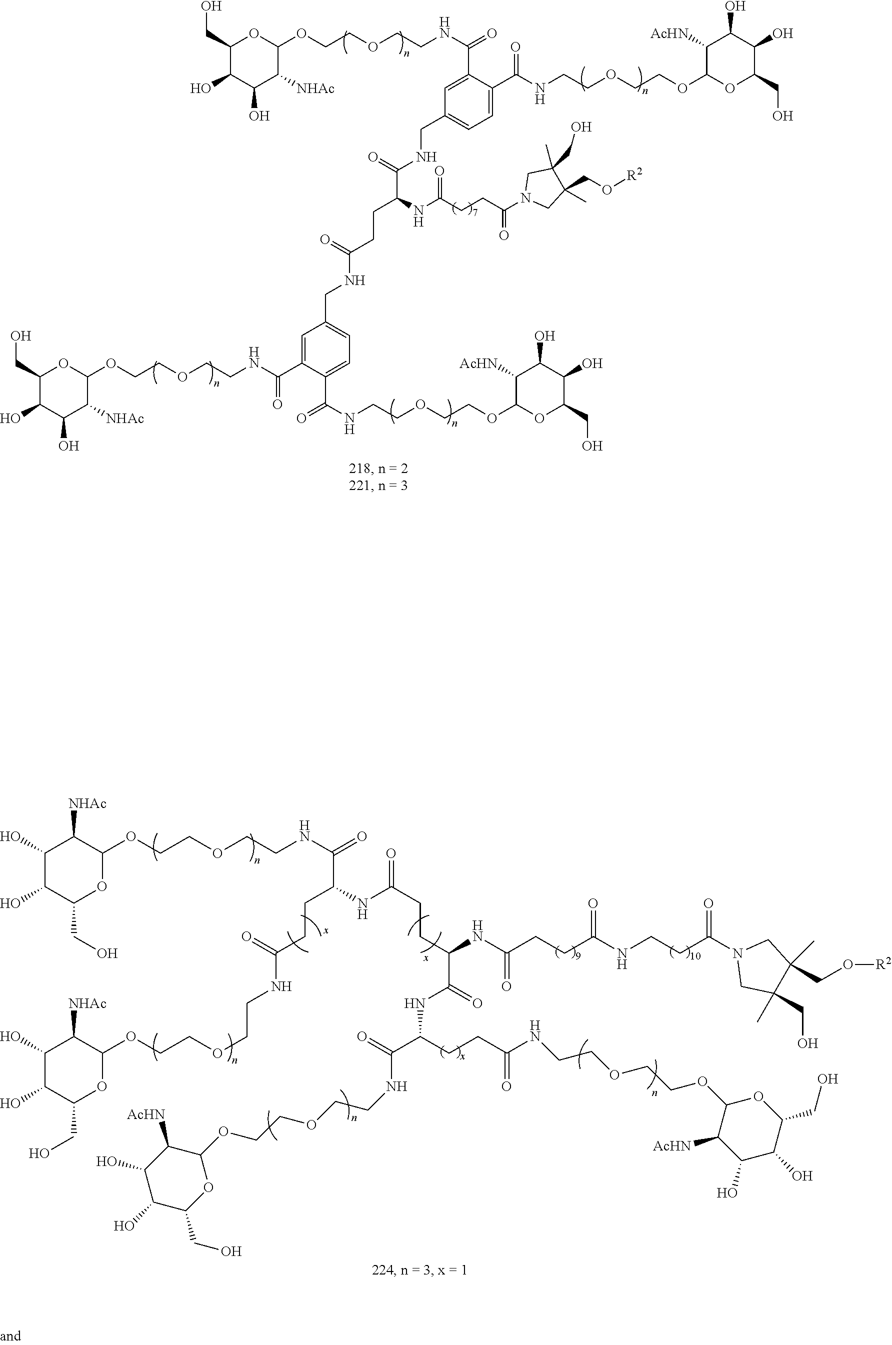 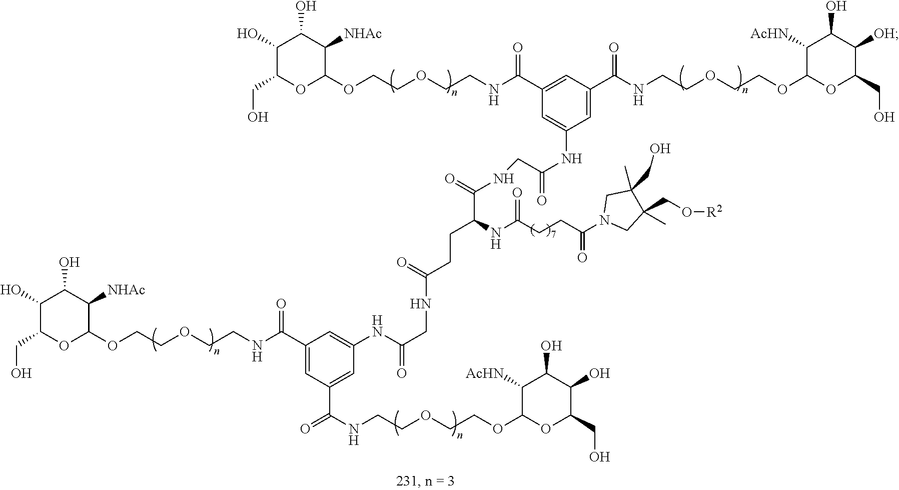
and pharmaceutically acceptable salts thereof.

7. The compound of claim 1, wherein the compound of formula (I) is: 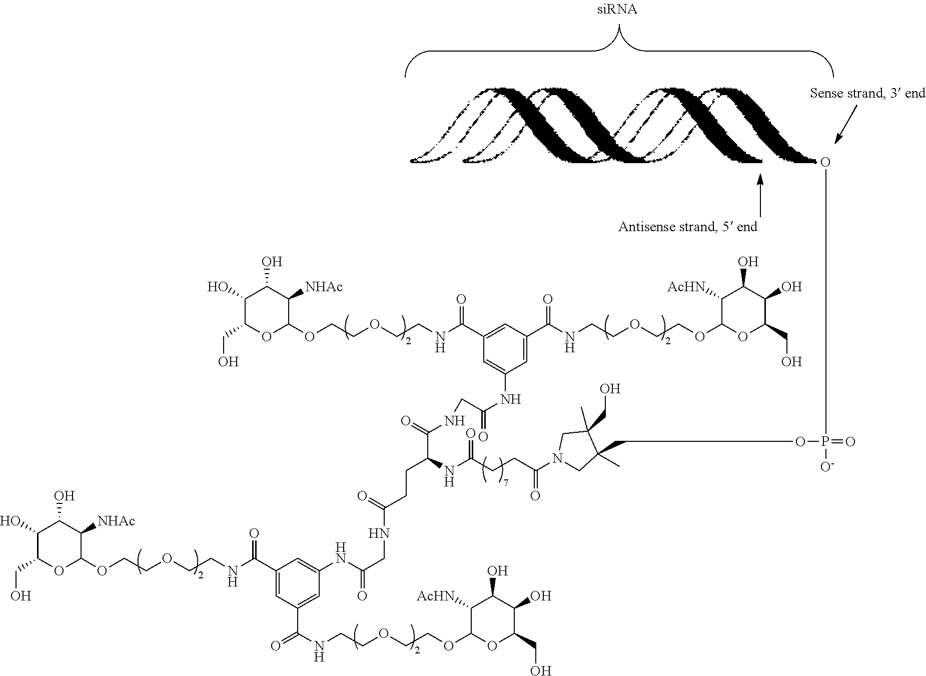
or a pharmaceutically acceptable salt thereof, wherein the siRNA depicted is selected from any one of siRNA 1-siRNA 28.

8. The compound of any one of claims 1-7, wherein the siRNA is selected from any one of siRNA 1-siRNA 24.

9. The compound of any one of claims 1-7, wherein the siRNA is selected from any one of siRNA 25-siRNA 28.

10. The compound of claim 9, wherein the siRNA is siRNA 25.

11. The compound of claim 9, wherein the siRNA is siRNA 26.

12. The compound of claim 9, wherein the siRNA is siRNA 27.

13. The compound of claim 9, wherein the siRNA is siRNA 28.

14. A method for treating acromegaly, comprising administering to a patient in need thereof an effective amount of the compound of any one of claims 1-13.

15. A method for reducing insulin-like growth factor-1 (IGF-1) in a patient, comprising administering to a patient in need thereof an effective amount of the compound of any one of claims 1-13.

16. A method for reducing growth hormone in a patient, comprising administering to a patient in need thereof an effective amount of the compound of any one of claims 1-13.

17. A method for reducing growth hormone receptor (GHR) in the liver in a patient, comprising administering to a patient in need thereof an effective amount of the compound of any one of claims 1-13.

18. The method of any one of claims 14-17, wherein the compound of formula (I) is administered subcutaneously.

19. A double stranded siRNA molecule selected from the group consisting of siRNA 1-siRNA 28.

20. A composition comprising a double stranded siRNA molecule of claim 19.
